(19) United States
(12) Patent Application Publication
(10) Pub. No.: US 2021/0238662 A1
(43) Pub. Date: Aug. 5, 2021

(54) PROBES AND METHODS OF USING SAME

(71) Applicant: 10x Genomics, Inc., Pleasanton, CA (US)

(72) Inventors: Felice Alessio BAVA, Pleasanton, CA (US); Patrick J. MARKS, San Francisco, CA (US)

(21) Appl. No.: 17/165,808

(22) Filed: Feb. 2, 2021

Related U.S. Application Data
(60) Provisional application No. 63/038,611, filed on Jun. 12, 2020.
(60) Provisional application No. 62/969,465, filed on Feb. 3, 2020.

(51) Int. Cl. C12Q 1/6832; C12Q 1/6841

(52) U.S. Cl. CPC C12Q 1/6832; C12Q 1/6841

(57) ABSTRACT

Provided herein are methods for analyzing a target nucleic acid, comprising contacting a target nucleic acid with a first polynucleotide and a second polynucleotide, and optionally one or more other polynucelotides such as a splint and/or a primer, to form a hybridization complex. In some embodiments, the first polynucleotide and the second polynucleotide are ligated to form a circular polynucleotide hybridized to the target nucleic acid, e.g., using DNA-templated ligation reaction(s). The circular polynucleotide and/or a product thereof (e.g., an RCA product) can be analyzed to analyze the target nucleic acid or a sequence thereof.

### CROSS-REFERENCE TO RELATED APPLICATIONS

This application claims priority to U.S. provisional application 62/969,465, filed Feb. 3, 2020, entitled “IN SITU SPATIAL ASSAYS,” and U.S. provisional application 63/038,611, filed Jun. 12, 2020, entitled “IN SITU PROBES AND METHODS OF USING SAME,” the contents of which are incorporated by reference in their entirety for all purposes.

### FIELD

The present disclosure relates in some aspects to methods for analyzing a target nucleic acid in a biological sample. In some aspects, the methods involve the use of a set of probe polynucleotides, for example comprising two, three, or more polynucleotides, for assessing target nucleic acids. In some aspects, the presence, amount and/or sequence of the target nucleic acid is analyzed in situ. In some aspects, the methods involve anchoring or linking a portion of the set of polynucleotides to a scaffold or other nucleic acids. Also provided are polynucleotides, set of polynucleotides, compositions, kits, devices and systems for use in accordance with the methods.

### BACKGROUND

Methods are available for analyzing nucleic acids present in a biological sample, such as a cell or a tissue. Current methods for analyzing nucleic acids present in a biological sample, for example for in situ analysis, can have low sensitivity and specificity and/or limited plexity, and can be biased, time-consuming, labor-intensive, and/or error-prone. Improved methods for analyzing nucleic acids present in a biological sample are needed. Provided herein are methods, polynucleotides, set of polynucleotides, compositions, and kits that meet such and other needs.

### BRIEF SUMMARY

In some embodiments, provided herein is a method for analyzing a target nucleic acid, comprising: contacting a target nucleic acid with a first polynucleotide and a second polynucleotide to form a hybridization complex, wherein: the first polynucleotide comprises docking region DR1, hybridization region HR1, and docking region DR1′, the second polynucleotide comprises docking region DR2, hybridization region HR2, and docking region DR2′, the target nucleic acid comprises hybridization regions HR1′ and HR2′, and HR1 and HR2 hybridize to HR1′ and HR2′, respectively; wherein the first polynucleotide comprises bridge region BR1 between DR1 and HR1 and/or the second polynucleotide comprises bridge region BR2 between DR2 and HR2; wherein the first polynucleotide comprises bridge region BR1′ between HR1 and DR1′ and/or the second polynucleotide comprises bridge region BR2′ between HR2 and DR2′; wherein DR1 and DR2 does not hybridize to the target nucleic acid; and wherein DR1 is connected (e.g., ligated) to DR2 and DR1′ is connected (e.g., ligated) to DR2′, whereby the first polynucleotide and the second polynucleotide form a circular polynucleotide hybridized to the target nucleic acid. In some embodiments, the first polynucleotide and/or the second polynucleotide comprises one or more barcode sequences.

In some aspects, provided herein are methods for analyzing a target nucleic acid, comprising: contacting a target nucleic acid with a first polynucleotide and a second polynucleotide to form a hybridization complex, wherein: the first polynucleotide comprises docking region DR1, bridge region BR1, hybridization region HR1, barcode sequence BCa1, and docking region DR1′, the second polynucleotide comprises docking region DR2, bridge region BR2, hybridization region HR2, one or both of barcode sequence BCb1 and bridge region BR2′, and docking region DR2′, the target nucleic acid comprises hybridization regions HR1′ and HR2′, and HR1 and HR2 hybridize to HR1′ and HR2′, respectively; wherein DR1 is connected (e.g., ligated) to DR2 and DR1′ is connected (e.g., ligated) to DR2′, whereby the first polynucleotide and the second polynucleotide form a circular polynucleotide hybridized to the target nucleic acid, which is analyzed to analyze the target nucleic acid or a sequence thereof.

In one aspect, disclosed herein is a method for analyzing a target nucleic acid, comprising contacting a target nucleic acid with a first polynucleotide and a second polynucleotide to form a hybridization complex, wherein the first polynucleotide comprises docking region DR1, bridge region BR1, hybridization region HR1, barcode sequence BCa1, and docking region DR1′; the second polynucleotide comprises docking region DR2, bridge region BR2, hybridization region HR2, one or both of barcode sequence BCb1 and bridge region BR2′, and docking region DR2′; the target nucleic acid comprises hybridization regions HR1′ and HR2′; and HR1 and HR2 hybridize to HR1′ and HR2′, respectively; wherein (i) the DR1/DR1′ hybridization and the DR2/DR2′ hybridization each forms a sticky end, or (ii) the DR1/DR1′ hybridization and the DR2/DR2′ hybridization each forms a blunt end; wherein the sticky ends or the blunt ends are connected (e.g., ligated), whereby the first polynucleotide and the second polynucleotide form a circular polynucleotide hybridized to the target nucleic acid, which is analyzed to analyze the target nucleic acid or a sequence thereof.

In one aspect, disclosed herein is a method for in situ gene sequencing of a target nucleic acid in a cell in an intact tissue, the method comprising: (a) contacting a fixed and permeabilized intact tissue with a first polynucleotide and a second polynucleotide, under conditions to allow for specific hybridization, to form a hybridization complex, wherein: the first polynucleotide comprises docking region DR1, bridge region BR1, hybridization region HR1, barcode sequence BCa1, and docking region DR1′, the second polynucleotide comprises docking region DR2, bridge region BR2, hybridization region HR2, one or both of barcode sequence BCb1 and bridge region BR2′, and docking region DR2′, the target nucleic acid comprises hybridization regions HR1′ and HR2′, and HR1 and HR2 hybridize to HR1′ and HR2′, respectively; wherein DR1 is connected (e.g., ligated) to DR2 and DR1′ is connected (e.g., ligated) to DR2′, whereby the first polynucleotide and the second polynucleotide form a circular polynucleotide hybridized to the target nucleic acid; (b) performing rolling circle amplification in the presence of a primer that hybridizes to the circular polynucleotide, wherein the performing comprises using the circular polynucleotide as a template for a polymerase to extend the primer and form one or more amplicons; (c) embedding the one or more amplicons in the presence of hydrogel subunits to form one or more hydrogel-embedded amplicons; (d) contacting the one or more hydrogel-embedded amplicons having the barcode sequence(s) with a pair of probes under conditions to allow for sequencing-by-ligation, wherein the ligation only occurs when both probes hybridize to the same amplicon; (e) reiterating step (d); and (f) imaging the one or more hydrogel-embedded amplicons to determine in situ gene sequencing of the target nucleic acid in the cell in the intact tissue.

In some of any such embodiments, in the hybridization complex formed with the first polynucleotide, second polynucleotide, and target nucleic acid, at least one region of the first polynucleptide and/or second polynucleotide does not hybridize to the target nucleic acid sequence between HR1′ and HR2′ of the target nucleic acid.

In any of the preceding embodiments, the first polynucleotide can comprise barcode sequences BCa1, . . . , and BCai, wherein i is an integer of 2 or greater. In any of the preceding embodiments, the first polynucleotide can further comprise bridge region BR1′. In any of the preceding embodiments, the second polynucleotide can comprise barcode sequence BCb1. In any of the preceding embodiments, the second polynucleotide can comprise bridge region BR2′. In any of the preceding embodiments, the second polynucleotide can comprise barcode sequences BCb1, . . . , and BCbj, wherein j is an integer of 2 or greater.

In any of the preceding embodiments, HR1′ can be between about 5 and about 35 nucleotides in length. In some embodiments, HR1′ is between about 8 and about 25 nucleotides in length, e.g., about 15 nucleotides in length. In some embodiments, HR1′ is about 15 nucleotides in length. In any of the preceding embodiments, HR2′ can be between about 5 and about 35 nucleotides in length. In some embodiments, HR2′ is between about 8 and about 25 nucleotides in length, e.g., about 15 nucleotides in length. In some embodiments, HR2′ is about 15 nucleotides in length. In any of the preceding embodiments, HR1′ and HR2′ can be substantially identical in length. In any of the preceding embodiments, HR1′ and HR2′ can be separated by 0 to 20 nucleotides. In any of the preceding embodiments, HR1 can be between about 5 and about 35 nucleotides in length. In some embodiments, HR1 is between about 8 and about 25 nucleotides in length, e.g., about 15 nucleotides in length. In some embodiments, HR1 is about 15 nucleotides in length.

In any of the preceding embodiments, BR1 and/or BR1′ can be between about 1 and about 20 nucleotides in length. In any of the preceding embodiments, BR1 and BR1′ can be between about 1 and about 20 nucleotides in length. In any of the preceding embodiments, BCa1, . . . , and/or BCai can be between about 5 and about 30 nucleotides in length, e.g., between about 10 and about 25 nucleotides in length. In any of the preceding embodiments, BCa1, BCa2, BCa3, . . . , and/or BCai can be between about 10 and about 25 nucleotides in length. In any of the preceding embodiments, at least one of BCa1, . . . , and/or BCai can identify the target nucleic acid or a sequence thereof, can be a unique identifier of a gene, can be an error-checking barcode, and/or can identify an mRNA as a splice variant and/or identify a splice junction sequence. In of the preceding embodiments, DR1 and/or DR1′ can be between about 1 and about 20 nucleotides in length. In any of the preceding embodiments, DR1 and DR1′ can be between about 1 and about 20 nucleotides in length. In any of the preceding embodiments, the first polynucleotide can be between about 10 and about 100 nucleotides in length.

In any of the preceding embodiments, HR2 can be between about 5 and about 35 nucleotides in length. In some embodiments, HR2 is between about 8 and about 25 nucleotides in length, e.g., about 15 nucleotides in length. In some embodiments, HR2 is about 15 nucleotides in length. In any of the preceding embodiments, BR2 and/or BR2′ can be between about 1 and about 20 nucleotides in length. In any of the preceding embodiments, BR2 and BR2′ can be between about 1 and about 20 nucleotides in length. In any of the preceding embodiments, BCb1, . . . , and/or BCbj can be between about 5 and about 30 nucleotides in length, e.g., between about 10 and about 25 nucleotides in length. In any of the preceding embodiments, BCb1, . . . , and/or BCbj can be between about 10 and about 25 nucleotides in length. In any of the preceding embodiments, at least one of BCb1, . . . , and/or BCbj can identify the target nucleic acid or a sequence thereof, can be a unique identifier of a gene, can be an error-checking barcode, and/or can identify an mRNA as a splice variant and/or identify a splice junction sequence. In any of the preceding embodiments, DR2 and/or DR2′ can be between about 1 and about 20 nucleotides in length. In any of the preceding embodiments, DR2 and DR2′ can be between about 1 and about 20 nucleotides in length. In any of the preceding embodiments, the second polynucleotide can be between about 10 and about 100 nucleotides in length.

In any of the preceding embodiments, the DR1/DR1′ hybridization and the DR2/DR2′ hybridization can each form a blunt end, and tailing (e.g., A-tailing) may be used to add one or more non-templated nucleotides to create a sticky end. In any of the preceding embodiments, the DR1/DR1′ hybridization and the DR2/DR2′ hybridization can each form a sticky end. In any of the preceding embodiments, the sticky ends may be directly or indirectly connected. In some embodiments, DR1 and DR2 form a first split hybridization region DR1-DR2, DR1′ and DR2′ form a second split hybridization region DR1′-DR2′, and DR1-DR2 and DR1′-DR2′ hybridize using each other as a splint. In some embodiments, DR1-DR2 comprises a nick between DR1 and DR2. In any of the preceding embodiments, DR1′-DR2′ can comprise a nick between DR1′ and DR2′. In any of the preceding embodiments, the method can further comprise, with or without gap filling, ligating DR1 and DR2 using DR1′ or DR2′ as a template, and/or with or without gap filling, ligating DR1′ and DR2′ using DR1 or DR2 as a template.

In any of the preceding embodiments, the method can further comprise ligating blunt ends formed by DR1/DR1′ hybridization and DR2/DR2′ hybridization. In any of the preceding embodiments, the method can further comprise ligating sticky ends formed by DR1/DR1′ hybridization and DR2/DR2′ hybridization, optionally wherein DR1 and DR2 form a first split hybridization region DR1-DR2, DR1′ and DR2′ form a second split hybridization region DR1′-DR2′, and DR1-DR2 and DR1′-DR2′ hybridize using each other as a splint. In any of the preceding embodiments, the method can further comprise ligating DR1 and DR2 and/or ligating DR1′ and DR2′ in the presence of a splint, wherein the splint facilitates ligation of DR1 to DR2 and ligation of DR1′ to DR2′. In any of the preceding embodiments, the method can further comprise ligating DR1 and DR2 and ligating DR1′ and DR2′ in the presence of a splint, wherein the splint facilitates ligation of DR1 to DR2 and ligation of DR1′ to DR2′.

In any of the preceding embodiments, DR1-DR2 can comprise a first gap between DR1 and DR2. In any of the preceding embodiments, DR1′-DR2′ can comprise a second gap between DR1′ and DR2′. In some embodiments, the first gap and/or the second gap are between about 1 and about 5 nucleotides in length. In some embodiments, the first gap and the second gap are between about 1 and about 5 nucleotides in length. In any of the preceding embodiments, the method can further comprise filling the first gap and ligating DR1 and DR2 using DR1′ or DR2′ as a template, and/or filling the second gap and ligating DR1′ and DR2′ using DR1 or DR2 as a template.

In any of the preceding embodiments, the first polynucleotide can comprise a DNA, an RNA, and/or a nucleic acid analog. In any of the preceding embodiments, the second polynucleotide can comprise a DNA, an RNA, and/or a nucleic acid analog. In any of the preceding embodiments, the target nucleic acid can comprise DNA, an RNA, and/or a nucleic acid analog. In any of the preceding embodiments, the target nucleic acid can be an mRNA.

In any of the preceding embodiments, the melting temperature (Tₘ) of HR1/HR1′ hybridization and the Tₘ of HR2/HR2′ hybridization are substantially the same. In any of the preceding embodiments, the melting temperature (Tₘ) of HR1/HR1′ hybridization and/or the Tₘ of HR2/HR2′ hybridization can be between about 40° C. and about 70° C. In any of the preceding embodiments, the melting temperature (Tₘ) of DR1/DR1′ hybridization, the Tₘ of DR2/DR2′ hybridization, and/or the Tₘ of DR1-DR2/DR1′-DR2′ hybridization can be lower than the Tₘ of HR1/HR1′ hybridization and/or the Tₘ of HR2/HR2′ hybridization. In any of the preceding embodiments, the melting temperature (Tₘ) of DR1/DR1′ hybridization, the Tₘ of DR2/DR2′ hybridization, and/or the Tₘ of DR1-DR2/DR1′-DR2′ hybridization can be lower than or to similar to room temperature, e.g., between about 16° C. and about 40° C. In any of the preceding embodiments, the melting temperature (Tₘ) of DR1/DR1′ hybridization, the Tₘ of DR2/DR2′ hybridization, and/or the Tₘ of DR1-DR2/DR1′-DR2′ hybridization can be between about 16° C. and about 40° C. In any of the preceding embodiments, the hybridization complex can be formed at a temperature higher than the melting temperature (Tₘ) of DR1/DR1′ hybridization, the Tₘ of DR2/DR2′ hybridization, and/or the Tₘ of DR1-DR2/DR1′-DR2′ hybridization, but lower than the Tₘ of HR1/HR1′ hybridization and/or the Tₘ of HR2/HR2′ hybridization. In any of the preceding embodiments, the hybridization complex can be formed at a temperature between about 30° C. and about 50° C., e.g., about 40° C. In any of the preceding embodiments, the hybridization complex can be formed at about 40° C. In any of the preceding embodiments, the hybridization complex can be formed at room temperature, e.g., between about 16° C. and about 40° C. In any of the preceding embodiments, the hybridization complex can be formed at a temperature between about 16° C. and about 40° C. In any of the preceding embodiments, the hybridization complex can be formed at or about the melting temperature (Tₘ) of DR1/DR1′ hybridization, the Tₘ of DR2/DR2′ hybridization, and/or the Tₘ of DR1-DR2/DR1′-DR2′ hybridization, e.g., within one, two, three, four, or five degrees above or below the melting temperature.

In any of the preceding embodiments, the method can further comprise a step of removing molecules that are not specifically hybridized to the target nucleic acid. In some embodiments, the removing step comprises one or more washes, e.g., a stringency wash. In some embodiments, the removing step comprises a stringency wash. In some embodiments, the removing step is performed at a temperature between or between about 10° C. and about 30° C., e.g., about 16° C. In some embodiments, the removing step is performed at room temperature.

In any of the preceding embodiments, formation of the circular polynucleotide can comprise a ligation reaction selected from the group consisting of enzymatic ligation, chemical ligation (e.g., click chemistry ligation), template dependent ligation, and/or template independent ligation. In some embodiments, the enzymatic ligation utilizes a ligase, e.g., a ligase having a DNA-splinted DNA ligase activity, such as a T4 DNA ligase. In some embodiments, the enzymatic ligation utilizes a ligase having a DNA-splinted DNA ligase activity. In some embodiments, the enzymatic ligation utilizes a T4 DNA ligase. In any of the preceding embodiments, the ligation reaction can be performed at a temperature lower than the temperature at which the hybridization complex is formed. In any of the preceding embodiments, the ligation reaction can be performed at a temperature lower than or similar to the melting temperature (Tₘ) of DR1/DR1′ hybridization, the Tₘ of DR2/DR2′ hybridization, and/or the Tₘ of DR1-DR2/DR1′-DR2′ hybridization, e.g., within one, two, three, four, or five degrees above or below the melting temperature. In some embodiments, the temperature at which the ligation reaction is performed is between about 10° C. and about 30° C., e.g., about 16° C. In some embodiments, the ligation reaction is performed at room temperature.

In any of the preceding embodiments, the method can further comprise a step of removing molecules that are not specifically hybridized in the hybridization complex after the ligation reaction. In some embodiments, the removing step comprises one or more washes, e.g., a stringency wash. In some embodiments, the removing step comprises a stringency wash.

In any of the preceding embodiments, the method can further comprise forming an amplification product using the circular polynucleotide as a template. In some embodiments, the amplification product is formed using isothermal amplification or non-isothermal amplification. In any of the preceding embodiments, the amplification product can be formed using rolling circle amplification (RCA). In some embodiments, the RCA comprises a linear RCA, a branched RCA, a dendritic RCA, or any combination thereof.

In any of the preceding embodiments, the splint can be used as a primer for forming the amplification product, and the circular polynucleotide can be used as a template for a polymerase to extend the primer and form the amplification product. In any of the preceding embodiments, the method can further comprise providing a primer for forming the amplification product, wherein the primer can hybridize to the circular polynucleotide, and the circular polynucleotide can be used as a template for a polymerase to extend the primer and form the amplification product.

In any of the preceding embodiments, the amplification product can be formed using a Phi29 polymerase. In any of the preceding embodiments, the amplification can be performed at a temperature lower than the melting temperature (Tₘ) of HR1/HR1′ hybridization and/or the Tₘ of HR2/HR2′ hybridization. In any of the preceding embodiments, the amplification can be performed at a temperature between about 15° C. and about 45° C., e.g., 30° C. or 35° C. In any of the preceding embodiments, the amplification can be performed at about 30° C. In any of the preceding embodiments, a sequence (e.g., a barcode sequence or complement thereof) in the amplification product can be determined, and the sequence may be indicative of the target nucleic acid or sequence thereof.

In any of the preceding embodiments, the target nucleic acid can be analyzed in situ in a tissue sample, e.g., a tissue section. In any of the preceding embodiments, the tissue sample can be a tissue section. In some embodiments, the tissue sample is an intact tissue sample or a non-homogenized tissue sample. In any of the preceding embodiments, the target nucleic acid can be in a cell in the tissue sample. In some embodiments, the methods further comprise permeabilizing and/or fixing the cell.

In any of the preceding embodiments, the tissue sample can be a fixed tissue sample, e.g., a formalin-fixed, paraffin-embedded (FFPE) sample, a frozen tissue sample, or a fresh tissue sample. In any of the preceding embodiments, the tissue sample can be embedded in a matrix, e.g., a hydrogel. In any of the preceding embodiments, the tissue sample can be embedded in a hydrogel. In some embodiments, the methods further comprise cross-linking the target nucleic acid and/or the amplification product to the matrix. In some embodiments, the methods further comprise cross-linking the target nucleic acid and the amplification product to the matrix.

In some embodiments, the determination comprises sequencing all or a portion of the amplification product and/or in situ hybridization to the amplification product. In some embodiments, the sequencing comprises sequencing hybridization, sequencing by ligation, and/or fluorescent in situ sequencing, and/or wherein the in situ hybridization comprises sequential fluorescent in situ hybridization. In any of the preceding embodiments, the determination can comprise hybridizing to the amplification product a detection oligonucleotide labeled with a fluorophore, an isotope, a mass tag, or a combination thereof. In any of the preceding embodiments, the determination can comprise imaging the amplification product. In any of the preceding embodiments, the target nucleic acid can be an mRNA, and the determination is performed when the target nucleic acid and/or the amplification product is in situ in the tissue sample.

Also provided herein are kits comprising a first polynucleotide and a second polynucleotide, wherein the first polynucleotide comprises docking region DR1, bridge region BR1, hybridization region HR1, barcode sequence BCa1, and docking region DR1′; the second polynucleotide comprises docking region DR2, bridge region BR2, hybridization region HR2, one or both of barcode sequence BCb1 and bridge region BR2′, and docking region DR2′; the first and second polynucleotides are capable of hybridizing to a target nucleic acid comprising hybridization regions HR1′ and HR2′, wherein HR1 and HR2 are capable of hybridizing to HR1′ and HR2′, respectively; and DR1 is capable of hybridizing to DR1′ and DR2 is capable of hybridizing to DR2′, wherein (i) the DR1/DR1′ hybridization and the DR2/DR2′ hybridization each forms a sticky end, or (ii) the DR1/DR1′ hybridization and the DR2/DR2′ hybridization each forms a blunt end. In some embodiments, the first and second polynucleotides are DNA molecules and the target nucleic acid is an mRNA molecule. In some embodiments, DR1 and DR2 does not hybridize to the target nucleic acid.

Also provided herein are compositions comprising a first polynucleotide and a second polynucleotide, wherein the first polynucleotide comprises docking region DR1, bridge region BR1, hybridization region HR1, barcode sequence BCa1, and docking region DR1′; the second polynucleotide comprises docking region DR2, bridge region BR2, hybridization region HR2, one or both of barcode sequence BCb1 and bridge region BR2′, and docking region DR2′; the first and second polynucleotides are hybridized to a target nucleic acid comprising hybridization regions HR1′ and HR2′, wherein HR1 and HR2 are hybridized to HR1′ and HR2′, respectively; and DR1 is hybridized to DR1′ and DR2 is hybridized to DR2′, wherein DR1 and DR2 does not hybridize to the target nucleic acid, wherein (i) the DR1/DR1′ hybridization and the DR2/DR2′ hybridization each forms a sticky end, or (ii) the DR1/DR1′ hybridization and the DR2/DR2′ hybridization each forms a blunt end.

In some embodiments, the kits and/or composition further comprise reagents for performing a ligation reaction and/or an amplification reaction. In some embodiments, the kits and/or composition further comprise reagents (e.g., detection probes, detectably labeled probe, or any intermediate probes provided herein) for detection of the first polynucleotide, second polynucleotide, or a product or derivative thereof.

In some embodiments, the first and second polynucleotides are DNA molecules and the target nucleic acid is an mRNA molecule. In any of the preceding embodiments, the composition can further comprise the target nucleic acid. In any of the preceding embodiments, the sticky ends can hybridize to each other. In some embodiments, the sticky ends are ligated to each other. In any of the preceding embodiments, the blunt ends can be ligated to each other.

### BRIEF DESCRIPTION OF THE DRAWINGS

FIG. 1 shows exemplary polynucleotides 101 and 102 that can form a circular polynucleotide used to analyze a nucleic acid sequence in an analayte, a nucleic acid sequence in a probe (e.g., a labelling agent) for the analyte, and/or a nucleic acid sequence in a product (e.g., RCA product) of the analyte and/or the probe.

FIG. 2 shows an exemplary method comprising hybridizing the polynucleotides of FIG. 1 to a target nucleic acid sequence, allowing the polynucleotides to hybridize to one another and ligating them to form a circular polynucleotide. The circular polynucleotide may be analyzed by one or more probes, including detection probes (e.g., fluorescently labelled detection probes) and/or intermediate probes that are detectable by detection probes. Alternatively, concurrently, or sequentially, the circular polynucleotide or a probe thereto may be amplified (e.g., using RCA) to generate an amplification product comprising multiple copies of the barcode sequences or complements thereof. The amplification product may be analyzed by one or more probes, including detection probes (e.g., fluorescently labelled detection probes) and/or intermediate probes that are detectable by detection probes.

FIG. 3 shows exemplary polynucleotides 301 and 302 that can form a circular polynucleotide, as well as a splint 303 that facilitates circularization of the polynucleotides upon hybridization to a nucleic acid sequence. The nucleic acid sequence can be in an analayte, in a probe (e.g., a labelling agent) for the analyte, and/or in a product (e.g., RCA product) of the analyte and/or the probe.

FIG. 4 shows an exemplary method comprising hybridizing the polynucleotides of FIG. 3 to a target nucleic acid sequence, allowing the polynucleotides to hybridize to a splint as a template for two ligation sites, and ligating the polynucleotides to form a circular polynucleotide. The circular polynucleotide may be analyzed by one or more probes, including detection probes (e.g., fluorescently labelled detection probes) and/or intermediate probes that are detectable by detection probes. Alternatively, concurrently, or sequentially, the circular polynucleotide or a probe thereto may be amplified (e.g., using RCA) to generate an amplification product comprising multiple copies of the barcode sequences or complements thereof. The amplification product may be analyzed by one or more probes, including detection probes (e.g., fluorescently labelled detection probes) and/or intermediate probes that are detectable by detection probes.

FIG. 5 shows exemplary circular probes hybridized to a target nucleic acid (e.g., an RNA transcript) assembled from two or more polynucleotides disclosed herein. The two or more polynucleotides collectively comprise a plurality of barcode sequences, and circular probes hybridizing to the same target nucleic acid may comprise different barcode sequences. The circular probes may be analyzed by one or more other probes, and/or amplified (e.g., using RCA) to generate an amplification product.

FIG. 6 shows the use of two exemplary polynucleotides as well as a splint in order to form a circular polynucleotide for analyzing a target nucleic acid. In this example, the splint spans one ligation site.

### DETAILED DESCRIPTION

Provided herein are methods for assembling composite padlock probes or circular probes for use in various applications, such as rolling circle amplification, e.g., for nucleic acid analysis such as in situ sequencing and/or in situ hybridization. In one embodiment, a plurality of polynucleotides are assembled, e.g., through hybridization followed by ligation, to form a padlock probe or circular probe. In some embodiments, the plurality of polynucleotides each contains (i) a hybridization region with which the polynucleotide hybridizes to a target nucleic acid of interest and (ii) a docking region. In one embodiment, the docking region is complementary to a splint, which may serve as a primer, for example for amplification of the formed padlock or circular probe. In another embodiment, the docking region of a polynucleotide is complementary to a second docking region in the polynucleotide such that the polynucleotide forms a structure similar to a hairpin loop. The docking regions of a polynucleotide may form a duplex comprising a sticky end, which may be connected to a sticky end formed by docking regions of another polynucleotide hybridized to the same or different nucleic acid of interest. In one embodiment, the hybridization region and docking region are separated by a bridge region, and that the docking region does not hybridize to the target nucleic acid of interest. As discussed herein, the organization and complementarity of the various regions of the polynucleotides provides structural components for the improved assembly of padlock or circular probes.

Traditional padlock probes comprise a linear DNA probe where the terminal ends of the probe are complementary to an internal sequence of a target molecule of interest. The nature of the complementarity brings the 5′ and 3′-ends of the probe sequence adjacent to each other such that the ends may be ligated to form a circle. This has a number of drawbacks that are addressed by the methods and polynucleotides described here.

One drawback of traditional padlock probes assembled by ligation occurs when the target nucleic acid of interest is a ribonucleic acid (RNA) molecule, such as an mRNA molecule. There is need for in situ transcriptomic tools for the spatial mapping of gene expression within tissues at cellular, or even subcellular resolution, including multiplexed in situ RNA hybridization and sequencing techniques. Rolling circle amplification of RNA sequences using padlock probes allows for amplification of the target sequence in a highly quantitative manner without relying on thermocyclers or other advanced read-out systems. However, the assembly of traditional padlock probes which are deoxynucleic acids (DNA) directly on an RNA template (e.g., without converting an RNA to cDNA) requires the use of an RNA-templated ligase to close the circle of a linear DNA probe to circularize the padlock. This ligation event is inefficient. While this efficiency can potentially be increased through the incorporation of ribonucleotides into DNA padlock probes, this requires the use of specialized RNA ligase enzymes (e.g., SplintR ligase) and can significantly increase the cost of manufacturing padlock probes, especially for multiplexed assays utilizing libraries of padlock probes.

The polynucleotide probes and methods of using them describe here are superior to previous methods because the docking regions of the polynucleotides allow for the use of a DNA-DNA templated ligation reaction(s), thus allowing for the use of a DNA-DNA templated ligase rather than an DNA-RNA or RNA-RNA templated ligase to circularize the probe complex. In some embodiments, the polynucleotide probes and methods provided herein do not use ribonucleotide(s) at or near to a ligation site. Although ribonucleotides may increase the efficiency of RNA-RNA templated or DNA-RNA templated ligation, DNA oligonucleotides containing ribonucleotides are expensive to make. For high-throughput multiplexed assays, thousands or even tens of thousands of such DNA oligonucleotides may need to be synthesized, making the assays cost prohibitive. In some embodiments, the polynucleotide probes and methods provided herein do not use an additional sequence 5′ to a target-specific binding site which is not hybridized to the target nucleic acid molecule upon hybridization of the probe to the target nucleic acid molecule and forms a 5′ flap containing one or more nucleotides at its 3′ end that is cleaved prior to ligation (e.g., using a 5′ Flap endonuclease (FEN) or other suitable enzyme with 5′ exonuclease activity).

Another drawback to traditional padlock probes is an upper level (e.g., upper bound) length limitation on the linear oligonucleotides that are circularized to form the padlock or circular probe. Limits on the length of such oligonucleotides are both functional and practical. Functionally, the longer an oligonucleotide sequence is, the more likely a sequence error is likely to result due to problems with the synthesis reaction. Errors in synthesis can lead to linear probes that are unable to circularize and thus are not able to serve as templates for rolling circle amplification. For example, assuming a linear oligonucleotide has a length of 100 bases, a synthesis error that occurs downstream of the initial sequence may produce a molecule that can hybridize to a target nucleic acid but is unable to circularize. In some examples, the synthesis error may result in a linear oligonucleotide of which the 5′ end can hybridize to a target nucleic acid (e.g., due to the 5′ end being synthesized first and thus being more accurate than the 3′ end), but the 3′ end contains a truncation or base error that abolishes templated ligation. In these examples, the erroneous padlock probes occupy the target nucleic acid but do not yield a successfully ligated circular probe for rolling circle amplification, leading to less efficient detection. Practically, synthesizing oligonucleotides significantly greater than 100 bases is typically not cost effective. In some embodiments, the polynucleotides described here address the length limitations of traditional padlock probes because multiple polynucleotides can be assembled into large padlock or circular probes, thus overcoming the bottleneck of oligonucleotide synthesis and making it possible to increase the barcoding space/potential within the padlock or circular probes.

### I. Polynucleotides

In some embodiments, the polynucleotide molecules provided here contain a first docking region (DR1) and a second docking region (DR1′), optionally a bridge region (BR), and a hybridization region (HR). In some embodiments, provided herein is a set of polynucleotides, in which a first polynucleotide comprises docking region DR1, hybridization region HR1, and docking region DR1′, and a second polynucleotide comprises docking region DR2, hybridization region HR2, and docking region DR2′. In some embodiments, HR1 and HR2 hybridize to hybridization regions HR1′ and HR2′, respectively. In some embodiments, hybridization regions HR1′ and HR2′ are adjacent to each other in the same polynucleotide molecule. In some embodiments, hybridization regions HR1′ and HR2′ are in different polynucleotide molecules.

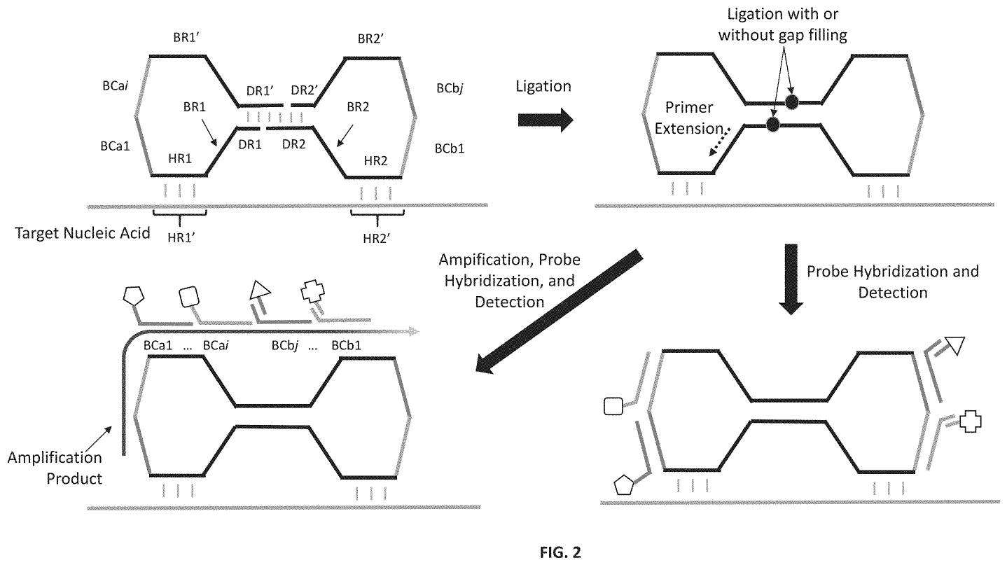

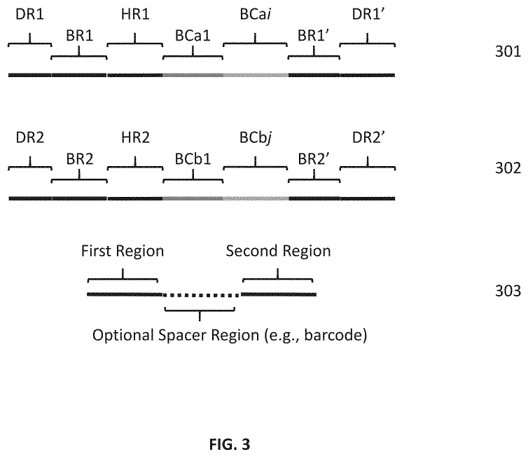

In some embodiments, the first polynucleotide comprises bridge region BR1 between DR1 and HR1. In some embodiments, the second polynucleotide comprises bridge region BR2 between DR2 and HR2. In some embodiments, the first polynucleotide comprises bridge region BR1′ between HR1 and DR1′. In some embodiments, the second polynucleotide comprises bridge region BR2′ between HR2 and DR2′. Not all bridge regions are necessary. For instance, in the example shown in FIG. 2, any one of bridge regions BR1, BR2, BR1′, and BR2′ may be omitted. In the example shown in FIG. 3, any one or two of bridge regions BR1, BR2, BR1′, and BR2′ may be omitted.

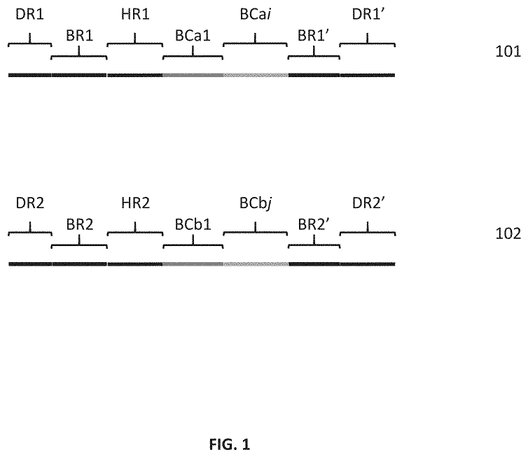

In some embodiments, the first polynucleotide and/or the second polynucleotide comprises one or more barcode sequences. The one or more barcode sequences may be between a hybridization region and a bridge region and/or between a bridge region and a docking region. In cases where there is no bridge region, the one or more barcode sequences may be between a hybridization region and a docking region, and may function as a bridge region to provide flexibility and permit hybridization of the docking region to another docking region or a splint. The one or more barcode sequences may also overlap with one another, and with any hybridization region, any bridge region, and/or any docking region of the polynucleotide. In some embodiments, a bridge region and/or a docking region of a polynucleotide disclosed herein comprises one or more barcode sequences. In some embodiments, provided herein is a plurality of pairs of first and second polynucleotides (e.g., 101 and 102 in FIG. 1, or 301 and 302 in FIG. 3). In some embodiments, different pairs of the first and second polynucleotides target different nucleic acid sequences, and each pair may comprise a bridge region and/or a docking region that correspond to a particular nucleic acid sequence to allow multiplexing. In some embodiments, two or more pairs of the first and second polynucleotides targeting different nucleic acid sequences may share one or more barcode sequences, one or more bridge region sequences, and/or one or more docking region sequences.

An exemplary arrangement of these components is shown in FIG. 1. For example, in some instances, the polynucleotide molecules described here comprise a first polynucleotide 101 and a second polynucleotide 102 that are configured to hybridize to a target molecule (such as an RNA molecule) and form a circular product comprising sequences of the first and second polynucleotide (e.g., by ligating the first and second polynucleotide together). Referring to FIG. 1, in some embodiments, the first polynucleotide 101 comprises a first docking region (DR1), a first bridge region (BR1), a hybridization region (HR1), a first barcode region (BCa1), optionally one or more additional barcode regions (BCai), wherein i is an integer number greater than or equal to zero, a second bridge region (BR1′), and a second docking region (DR1′). The barcode sequence BCai and bridge region (e.g., BR1 and/or BR1′) are optional components of the polynucleotide and may be included or omitted in any combination. With continued reference to FIG. 1, in some instances, the second polynucleotide 102 comprises a first docking region (DR2), a first bridge region (BR2), a hybridization region (HR2), a first barcode region (BCb1), optionally one or more additional barcode regions (BCbj), wherein j is an integer number greater than or equal to zero, a second bridge region (BR2′), and a second docking region (DR2′). The barcode sequence BCbj and bridge regions (e.g., BR2 and/or BR2′) are optional components of the polynucleotide and may be included or omitted in any combination. In any of the embodiments disclosed herein, the values of i and j can be selected independent of each other.

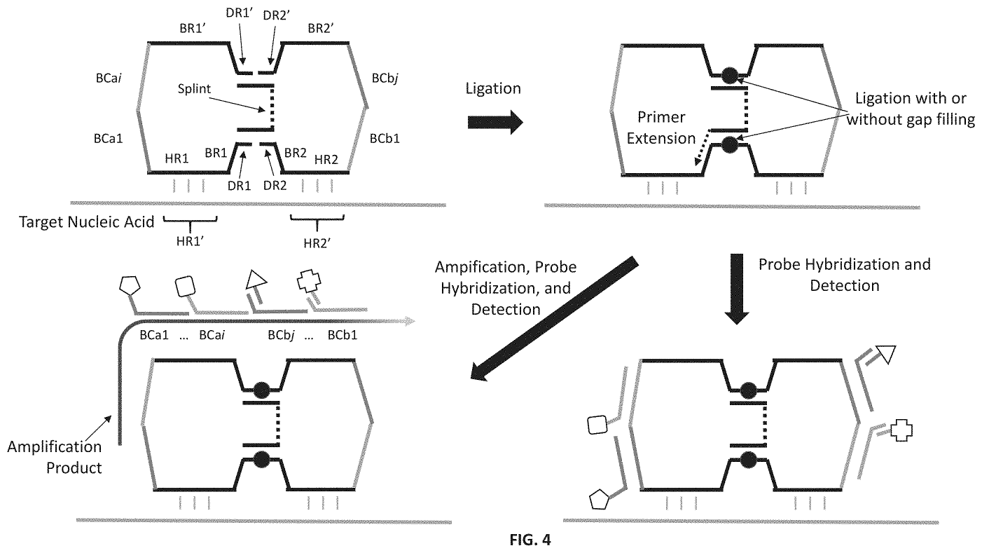

In some of any such embodiments, HR1 and HR2 are internal sequences of the first and second polynucleodetide, respectively. For example, HR1 of the first polynucleotide and HR2 of the second polynucleotide are each flanked by sequences of the respective polynucleotide on both sides. In some of any such embodiments, the docking regions are positioned as end sequences (e.g., positioned at the 3′ and/or the 5′ end) of the first and second polynucleodetide, respectively. In some cases, both ends of the first polynucletide is a docking region (e.g., DR1 and DR1′) and both ends of the second polynucletide is a docking region (e.g., DR2 and DR2′). In some of any embodiments herein, a docking region herein (e.g., DR1, DR2, DR1′, and DR2′) does not hybridize to the target nucleic acid. For example, as shown in FIG. 2 and FIG. 4, DR1 and DR2 in some examples do not hybridize to the target nucleic acid (e.g., RNA). In some of any embodiments herein, a docking region herein (e.g., DR1, DR2, DR1′, and DR2′) comprises one or more subregions that do not hybridize to the target nucleic acid, while one or more other subregions may hybridize to the target nucleic acid. In some embodiments, the ligation between docking regions (e.g., between DR1 and DR2) may be templated on a nucleic acid (e.g., DR1′ and/or DR2′, or a splint) other than a sequence of the target nucleic acid. In some embodiments, the ligation between docking regions (e.g., between DR1 and DR2) may not depend on the docking region(s) hybridizing to the target nucleic acid.

Furthermore, the arrangements of features shown in FIG. 1 may be 5′ to 3′ or 3′ to 5′. The first docking region (e.g., DR1 or DR2) is a 3′ or 5′ end sequence that can be complementary to all or a portion of the second docking region in the same polynucleotide (e.g., DR1′ or DR2′) or to a splint (see, e.g., FIG. 3). As shown in FIG. 3, the splint can comprise a first region that is complementary to the docking region (e.g., DR1 or DR2), a second region that is complementary to the second docking region (e.g., DR1′ or DR2′), and optionally a spacer region between the first and second regions of the splint. In some embodiments, the spacer region may be detected, e.g., via hybridization to one or more probes (e.g., a detectably labeled probe or an intermediate probe), as a control for whether the first and second polynucleotides are in proximity to each another and/or whether the first and second polynucleotides are specifically hybridized to the target sequences.

In some embodiments, the splint functions to facilitate ligation of the first and second polynucleotides. In some instances, the splint may also function as a primer, for example, in a primer extension reaction (e.g., RCA) using a circular probe formed from the first and second polynucleotide.

In some embodiments, a splint disclosed herein comprises one or more barcode sequences. In some embodiments, the spacer region of a splint comprises one or more barcode sequences. In some embodiments, different pairs of the first and second polynucleotides target different nucleic acid sequences, and each pair may hybridize to a splint that corresponds to a particular nucleic acid sequence to allow multiplexing. In some embodiments, two or more pairs of the first and second polynucleotides targeting different nucleic acid sequences may share a splint. In some embodiments, two or more pairs of the first and second polynucleotides targeting different nucleic acid sequences may hybridize to different splints that share a spacer region sequence.

The first docking regions and the second docking regions provide sequences to promote ligation events (e.g., between the first and second polynucleotides) that serve to circularize the polynucleotide(s) into a circular molecule. In some embodiments, the bridge region is disposed between the hybridization region and the docking region, and acts as a spacer between these regions. Preferably the bridge region is sufficiently long as to separate the docking region from the hybridization region so that the ends of the polynucleotide are available for subsequent ligation.

Polynucleotides can be any suitable length, but in some instances are between about 20 and about 200 nucleotides in length. In some embodiments, the first polynucleotide and/or the second polynucleotide, independent of each other, are between about 10 and about 20, between about 20 and about 25, between about 25 and about 30, between about 30 and about 35, between about 35 and about 40, between about 40 and about 45, between about 45 and about 50, between about 50 and about 60, between about 60 and about 70, between about 70 and about 80, between about 80 and about 90, between about 90 and about 100, between about 100 and about 110, between about 110 and about 120, between about 120 and about 130, between about 130 and about 140, between about 140 and about 150, between about 150 and about 160, between about 160 and about 170, between about 170 and about 180, between about 180 and about 190, or between about 190 and about 200 nucleotides in length.

FIG. 2 shows the assembly of two polynucleotides (see, e.g., FIG. 1) hybridized to a target nucleic acid of interest, for example an RNA molecule, to form a hybridization complex. As shown in FIG. 2, a first polynucleotide (e.g., polynucleotide 101 of FIG. 1) comprising a docking region DR1, bridge region BR1, hybridization region HR1, a barcode sequence BCa1 and docking region DR1′ is hybridized to a region of the target nucleic acid of interest (HR1′). The figure shows a second polynucleotide (e.g., polynucleotide 102 of FIG. 1) comprising a docking region DR2, bridge region BR2, hybridization region HR2, and docking region DR2′ hybridized to a second region of the target nucleic acid of interest (HR2′). For example, a first polynucleotide (e.g., polynucleotide 101 of FIG. 1) comprises a docking region DR1′ which is complementary to docking region DR1 and to at least a portion of DR2 on a second polynucleotide (e.g., polynucleotide 102 of FIG. 1). Similarly, the second polynucleotide (e.g., polynucleotide 102 of FIG. 1) comprises a docking region DR2 which is complementary to docking region DR2′ and to at least a portion of DR1′ on the first polynucleotide (e.g., polynucleotide 101 of FIG. 1). In another example (not show in FIG. 2), a first polynucleotide (e.g., polynucleotide 101 of FIG. 1) comprises a docking region DR1 which is complementary to docking region DR1′ and to at least a portion of DR2′ on a second polynucleotide (e.g., polynucleotide 102 of FIG. 1), while the second polynucleotide (e.g., polynucleotide 102 of FIG. 1) comprises a docking region DR2′ which is complementary to docking region DR2 and to at least a portion of DR1 on the first polynucleotide (e.g., polynucleotide 101 of FIG. 1). The regions BCai, BR1, BR1′, BR2, BR2′, BCb1, and BCbj are optional components of the polynucleotides and may be included or omitted in any combination.

In some instances, HR1′ and HR2′ are directly adjacent on the target nucleic acid and form a contiguous sequence. In other instances, HR1′ and HR2′ are not contiguous on the target nucleic acid (e.g., HR1′ and HR2′ are separated by one or more bases). In some embodiments, HR1′ and HR2′ are separated by one base, two bases, three bases, four bases, five bases, six bases, seven bases, eight bases, nine bases, 10 bases, or more than 10 bases. In some embodiments, HR1′ and HR2′ are separated by about 10 bases, about 15 bases, about 20 bases, about 25 bases, about 30 bases, about 35 bases, about 40 bases, about 45 bases, about 50 bases, about 55 bases, about 60 bases, about 65 bases, about 70 bases, about 75 bases, about 80 bases, about 85 bases, about 90 bases, about 95 bases, about 100 bases, or more than about 100 bases.

With continued reference to FIG. 2, in some embodiments, all or a portion of DR1 and DR1′ are complementary to one another and hybridize to form a hairpin loop-like structure. The embodiment illustrated in FIG. 2 shows DR1 and DR1′ forming an overhang once hybridized. In an alternative embodiment not shown in FIG. 2, the docking regions do not form an overhanging region. In both embodiments, the hybridization of DR1 and DR1′ and DR2 and DR2′ facilitates the ligation (see, e.g., FIG. 2) of the two polynucleotides by providing the 5′ and 3′-ends for a ligation reaction. In some embodiments, both polynucleotides are composed of deoxyribonucleic acids and as such, a DNA-DNA templated ligase catalyzes the ligation reaction, whether it is a “sticky end” or a blunt end ligation. The 5′ and 3′-ends of the docking regions may lie directly adjacent to one another, or there may be a nick or gap between the ends that may require gap filling. In some embodiments, there are nicks between DR1 and DR1′ and between DR2 and DR2′. In some embodiments, there are gaps between DR1 and DR1′ and between DR2 and DR2′. In some embodiments, one of DR1-DR1′ and DR2-DR2′ is separated by a nick while the other is separated by a gap. In some embodiments, the gap between DR1 and DR2 and the gap between DR1′ and DR2′ are, independent of each other, between 1 and 10 nucleotides in length. In some embodiments, the gap between DR1 and DR2 and the gap between DR1′ and DR2′, independent of each other, are one, two, three, four, five, six, seven, eight, nine, 10, or more than 10 nucleotides in length. In an embodiment comprising a plurality of polynucleotides, both types of junctions may be present and gaps of different lengths can also be present.

In some embodiments, bridge regions BR1 and BR2 separate docking regions DR1 and DR2 from hybridization regions HR1 and HR2, respectively. Without being limited to any particular mechanism of action, it is thought the separation provided by the bridge regions serves to separate the ends of the polynucleotides from the target nucleic acid and bring the docking regions into close proximity to facilitate DNA-DNA templated ligation. In some embodiments, the ligations of DR1 to DR2 and DR1′ to DR2′ are catalyzed by a ligase having a DNA-DNA templated ligase activity. In some instances, the first and/or second polynucleotide comprise a 5′ phosphate to facilitate ligation. In some instances, the first and/or second polynucleotide comprise a 3′-OH group to facilitate ligation. In some embodiments, the 5′ end of DR1′ comprises a phosphate group and/or the 3′ end of DR2′ comprises a hydroxyl group, and/or the 5′ end of DR2 comprises a phosphate group and/or the 3′ end of DR1 comprises a hydroxyl group. In some other embodiments, the 5′ end of DR2′ comprises a phosphate group and/or the 3′ end of DR1′ comprises a hydroxyl group, and/or the 5′ end of DR1 comprises a phosphate group and/or the 3′ end of DR2 comprises a hydroxyl group.

In some instances, one or both of DR1/DR1′ hybridization and DR2/DR2′ hybridization form a blunt end. Blunt ends are ligatable without an adaptor. In some embodiments, a suitable ligation adaptor may be used, and the blunt ends may be modified to provide a sequence that is complementary to the ligation adaptor, including, but not limited to a T-overhang, an A-overhang, a CG overhang, or any other ligatable sequence.

In some embodiments, each of the two polynucleotides is first hybridized to the target nucleic acid at a temperature higher than the melting temperatures of docking region hybridization. After removing unhybridized and/or nonspecifically hybridized polynucleotides (e.g., through a stringent wash), the temperature is lowered to about the melting temperature of docking region hybridization, e.g., within about 5, 4, 3, 2, or 1 degree above or below the melting temperature of docking region hybridization. The docking region hybridizations bring into close proximity with one another the free ends of DR1 and DR2 and the free ends of DR1′ and DR2′. The free ends of DR1 and DR2, as well as the free ends of DR1′ and DR2′, can then be ligated to one another either with or without gap filling, thus forming a circular polynucleotide hybridized to the target nucleic acid. RCA of the circular polynucleotide can then be initiated.

In some embodiments, each of the two polynucleotides is first hybridized to the target nucleic acid at a temperature higher than the melting temperatures of docking region hybridization. After removing unhybridized and/or nonspecifically hybridized polynucleotides (e.g., through a stringent wash), the temperature is lowered to below the melting temperature of docking region hybridization. The docking region hybridizations bring into close proximity with one another the free ends of DR1 and DR2 and the free ends of DR1′ and DR2′. The free ends of DR1 and DR2, as well as the free ends of DR1′ and DR2′, can then be ligated to one another either with or without gap filling, thus forming a circular polynucleotide hybridized to the target nucleic acid. RCA of the circular polynucleotide can then be initiated.

In some embodiments, a target nucleic acid is contacted with each of the two polynucleotides at a temperature lower than the melting temperatures of docking region hybridization. At this temperature, the two polynucleotides hybridize to the target nucleic acid. In addition, the docking regions also hybridize, bringing into close proximity with one another the free ends of DR1 and DR2 and the free ends of DR1′ and DR2′. Unhybridized and/or nonspecifically hybridized polynucleotides may be removed, e.g., through a stringent wash. The free ends of DR1 and DR2, as well as the free ends of DR1′ and DR2′, can then be ligated to one another either with or without gap filling, thus forming a circular polynucleotide hybridized to the target nucleic acid. RCA of the circular polynucleotide can then be initiated.

FIG. 3 and FIG. 4 show an alternative embodiment that utilizes a splint to facilitate the ligation of the polynucleotides. As shown in FIG. 3, the arrangement of the various components (e.g., DR1, BR1, DR1′, etc.) in polynucleotides 301 and 302 is similar to the arrangement shown in FIGS. 1-2, however DR1 and DR1′ are not complementary to one another in this embodiment. Instead, these regions as well as DR2 and DR2′, which are similarly not complementary to one another, are complementary to regions of the splint. As shown in FIG. 4, the splint 303 can comprise a first region that is complementary to the docking region (e.g., DR1 and/or DR2), a second region that is complementary to the second docking region (e.g., DR1′ and/or DR2′), and optionally a spacer region between the first and second regions of the splint. The splint may be a single oligonucleotide molecule, or may be provided as two or more oligonucleotide molecules. The hybridization of DR1, DR1′, DR2, and DR2′ to the splint allows it to facilitate the ligation of the two polynucleotides. In some embodiments, the two polynucleotides provide the 5′ and 3′-ends for a ligation reaction, which is facilitated by the splint and catalyzed by a ligase having a DNA-DNA templated ligase activity. In some embodiments, the splint does not comprise ribonucleotide(s). For example, in some instances, the splint comprises (1) a first region complementary to at least a portion of DR1′ and at least a portion of DR2′; and (2) a second region complementary to at least a portion of DR1 and at least a portion of DR2. As such, the splint functions to bring the 5′ and 3′ ends of the first and second polynucleotide (e.g., polynucleotides 101 and 102 of FIG. 1 or polynucleotides 301 and 302 of FIG. 3) in close proximity of one another for a DNA-DNA templated ligation. In some embodiments, the first complementary region and/or the second complementary region are at the 5′ or 3′ end of the splint. In other embodiments, the first complementary region and/or the second complementary region are an internal sequence of the splint. In some instances, the splint comprises a spacer region between the first and second complementary regions. In some embodiments, the spacer region is not complementary to the first polynucleotide, the second polynucleotide, and/or the target nucleic acid. In some embodiments, the spacer region is zero, one, two, three, four, five, six, seven, eight, nine, 10, or more than 10 nucleotides in length. The arrangements of features shown in FIG. 3 and FIG. 4 may be 5′ to 3′ or 3′ to 5′. For instance, each docking region DR, DR1′, DR2, or DR2′ can be a 3′ or 5′ end sequence that is complementary to a portion of the splint.

Thus, disclosed herein in some embodiments are probes that hybridize to an analyte (e.g., a target nucleic acid). In some instances, the probes hybridize to adjacent sequences (e.g., hybridization regions HR1′ and HR2′) on an analyte (e.g., a target nucleic acid). In some embodiments, HR1′ and R2′ of the analyte (e.g., target nucleic acid) are separated by a region which does not hybridize to any part of the first or second polynucleotide. In some cases, DR1 and DR2 of the first and second polynucleotide does not hybridize to the target nucleic acid. In some cases, DR1 and DR2 of the first and second polynucleotide does not comprise any subregion that hybridizes to the target nucleic acid, e.g., to the region separating the HR1′ and R2′ of the target nucleic acid. The probes disclosed herein include a first probe oligonucleotide (e.g., a first polynucleotide), a second probe oligonucleotide (e.g., a second polynucleotide), and a third probe oligonucleotide (e.g., a splint).

### (i) First Probe Oligonucleotide (e.g., First Polynucleotide)

Disclosed herein in some embodiments are methods of generating a ligation product. To generate a ligation product, a first probe oligonucleotide (e.g., a first polynucleotide) is used. In some embodiments, a first probe oligonucleotide (e.g., a first polynucleotide) hybridizes to an analyte (e.g., a target nucleic acid). In some instances, the first probe oligonucleotide (e.g., the first polynucleotide) is a sequence that is at least 40 nucleotides, at least 45 nucleotides, at least 50 nucleotides, at least 55 nucleotides, at least 60 nucleotides, at least 65 nucleotides, at least 70 nucleotides, at least 75 nucleotides, at least 80 nucleotides, at least 85 nucleotides, at least 90 nucleotides, at least 95 nucleotides, at least 100 nucleotides, or longer.

In some instances, a first probe oligonucleotide (e.g., a first polynucleotide) includes a sequence (e.g., HR1) that is fully (e.g., 100%) complementary to a sequence of an analyte (e.g., HR1′ of a target nucleic acid). In some instances, a first probe oligonucleotide (e.g., a first polynucleotide) includes a sequence (e.g., HR1) that is partially complementary to a sequence of an analyte (e.g., HR1′ of a target nucleic acid). Partially complementary includes at least 70%, at least 75%, at least 80%, at least 85%, at least 90%, at least 91%, at least 92%, at least 93%, at least 94%, at least 95%, at least 96%, at least 97%, at least 98%, or at least 99% complementary to a sequence of an analyte (e.g., HR1′ of a target nucleic acid).

In some instances, the first probe oligonucleotide (e.g., the first polynucleotide) includes a first barcode sequence (e.g., barcode sequence BCa1). In some instances, the first barcode sequence (e.g., barcode sequence BCa1) provides a sequence for hybridization of an oligonucleotide having one or more detectable moieties (e.g., a detection probe). In some instances, the first barcode sequence (e.g., barcode sequence BCa1) is fully complementary to an oligonucleotide having one or more detectable moieties (e.g., a detection probe). In some instances, the first barcode sequence (e.g., barcode sequence BCa1) is partially (e.g., at least 70%, at least 75%, at least 80%, at least 85%, at least 90%, at least 91%, at least 92%, at least 93%, at least 94%, at least 95%, at least 96%, at least 97%, at least 98%, or at least 99%) complementary to an oligonucleotide having one or more detectable moieties (e.g., a detection probe).

In some instances, the first probe oligonucleotide (e.g., the first polynucleotide) includes a second barcode sequence (e.g., BCai, wherein i is an integer of 2 or greater). In some instances, the second barcode sequence (e.g., BCai) provides a sequence for hybridization of an oligonucleotide having one or more detectable moieties (e.g., a detection probe). In some instances, the second barcode sequence (e.g., BCai) is fully complementary to an oligonucleotide having one or more detectable moieties (e.g., a detection probe). In some instances, the second barcode sequence (e.g., BCai) is partially (e.g., at least 70%, at least 75%, at least 80%, at least 85%, at least 90%, at least 91%, at least 92%, at least 93%, at least 94%, at least 95%, at least 96%, at least 97%, at least 98%, or at least 99%) complementary to an oligonucleotide having one or more detectable moieties (e.g., a detection probe).

In some instances, the first probe oligonucleotide (e.g., the first polynucleotide) includes one barcode sequence (e.g., barcode sequence BCa1). In some instances, the first probe oligonucleotide (e.g., the first polynucleotide) includes at least two barcode sequences (e.g., barcode sequence BCa1 and BCa2). In some instances, the first probe oligonucleotide (e.g., the first polynucleotide) includes at least three, at least four, at least five, or more barcode sequences. The barcodes enable transcriptome-level multiplexing potential by methods of sequential hybridization of oligonucleotides that have a detectable moiety to one or more barcode sequence.

In some instances, the first probe oligonucleotide (e.g., the first polynucleotide) includes a first docking sequence (e.g., DR1) that is a first docking site for hybridization of a third oligonucleotide (e.g., a splint). In some instances, the first docking sequence (e.g., DR1) is fully complementary to a sequence (e.g., a second region) of a third probe oligonucleotide (e.g., a splint). In some instances, the first docking sequence (e.g., DR1) is partially (e.g., at least 70%, at least 75%, at least 80%, at least 85%, at least 90%, at least 91%, at least 92%, at least 93%, at least 94%, at least 95%, at least 96%, at least 97%, at least 98%, or at least 99%) complementary to a sequence (e.g., a second region) of a third probe oligonucleotide (e.g., a splint).

In some instances, the first probe oligonucleotide (e.g., the first polynucleotide) includes a second docking sequence (e.g., DR1′) that is a second docking site for hybridization of a third probe oligonucleotide (e.g., a splint) to the first probe oligonucleotide (e.g., the first polynucleotide). In some instances, the first docking sequence (e.g., DR1) and the second docking sequence (e.g., DR1′) are at opposite (e.g., 5′ or 3′) ends of the first probe oligonucleotide (e.g., the first polynucleotide).

In some instances, the first probe oligonucleotide (e.g., the first polynucleotide) includes, in order from 5′ to 3′: a first docking sequence (e.g., DR1), a sequence that is complementary to a sequence of an analyte (e.g., HR1 complementary to HR1′ of a target nucleic acid), one or more barcode sequences (e.g., BCa1, . . . , and BCai), and a second docking sequence (e.g., DR1′). In some instances, the first probe oligonucleotide (e.g., the first polynucleotide) includes, in order from 5′ to 3′: a second docking sequence (e.g., DR1′), one or more barcode sequences (e.g., BCa1, . . . , and BCai), a sequence that is complementary to a sequence of an analyte (e.g., HR1 complementary to HR1′ of a target nucleic acid), and a first docking sequence (e.g., DR1).

### (ii) Second Probe Oligonucleotide (e.g., Second Polynucleotide)

Also disclosed herein is a second probe oligonucleotide (e.g., a second polynucleotide). In some embodiments, a second probe oligonucleotide (e.g., a second polynucleotide)hybridizes to an analyte (e.g., a target nucleic acid). In some instances, the second probe oligonucleotide (e.g., the second polynucleotide) is a sequence that is at least 40 nucleotides, at least 45 nucleotides, at least 50 nucleotides, at least 55 nucleotides, at least 60 nucleotides, at least 65 nucleotides, at least 70 nucleotides, at least 75 nucleotides, at least 80 nucleotides, at least 85 nucleotides, at least 90 nucleotides, at least 95 nucleotides, at least 100 nucleotides, or longer.

In some instances, the first probe oligonucleotide (e.g., the first polynucleotide) and the second probe oligonucleotide (e.g., the second polynucleotide) hybridize to sequences (e.g., HR1′ and HR2′) that are immediately adjacent to one another on the same analyte (e.g., on the same target nucleic acid). In some instances, the first probe oligonucleotide (e.g., the first polynucleotide) and the second probe oligonucleotide (e.g., the second polynucleotide) hybridize to sequences that are at least 5 at least 10, at least 15, at least 20, at least 25, at least 30, at least 35, at least 40, at least 45, at least 50, at least 75, at least 100, or more nucleotides apart.

In some instances, the second probe oligonucleotide (e.g., the second polynucleotide) includes a sequence (e.g., HR2) that is fully (e.g., 100%) complementary to a sequence of an analyte (HR2′ of a target nucleic acid). In some instances, the second probe oligonucleotide (e.g., the second polynucleotide) includes a sequence (e.g., HR2) that is partially complementary to a sequence of an analyte (e.g., HR2′ of a target nucleic acid). Partially complementary includes at least 70%, at least 75%, at least 80%, at least 85%, at least 90%, at least 91%, at least 92%, at least 93%, at least 94%, at least 95%, at least 96%, at least 97%, at least 98%, or at least 99% complementary to a sequence of an analyte (e.g., HR2′ of a target nucleic acid).

In some instances, the second probe oligonucleotide (e.g., the second polynucleotide) includes a third barcode sequence (e.g., BCb1). In some instances, the second barcode sequence (e.g., BCb1) provides a sequence for hybridization of an oligonucleotide having one or more detectable moieties (e.g., a detection probe). In some instances, the third barcode sequence (e.g., BCb1) is fully complementary to an oligonucleotide having one or more detectable moieties (e.g., a detection probe). In some instances, the third barcode sequence (e.g., BCb1) is partially (e.g., at least 70%, at least 75%, at least 80%, at least 85%, at least 90%, at least 91%, at least 92%, at least 93%, at least 94%, at least 95%, at least 96%, at least 97%, at least 98%, or at least 99%) complementary to an oligonucleotide having one or more detectable moieties (e.g., a detection probe).

In some instances, the second probe oligonucleotide (e.g., the second polynucleotide) includes a fourth barcode sequence (e.g., BCbj, wherein j is an integer of 2 or greater). In some instances, the fourth barcode sequence (e.g., BCbj) provides a sequence for hybridization of an oligonucleotide having one or more detectable moieties (e.g., a detection probe). In some instances, the fourth barcode sequence (e.g., BCbj) is fully complementary to an oligonucleotide having one or more detectable moieties (e.g., a detection probe). In some instances, the fourth barcode sequence (e.g., BCbj) is partially (e.g., at least 70%, at least 75%, at least 80%, at least 85%, at least 90%, at least 91%, at least 92%, at least 93%, at least 94%, at least 95%, at least 96%, at least 97%, at least 98%, or at least 99%) complementary to an oligonucleotide having one or more detectable moieties (e.g., a detection probe).

In some instances, the second probe oligonucleotide (e.g., the second polynucleotide) includes one barcode sequence (e.g., BCb1). In some instances, the second probe oligonucleotide (e.g., the second polynucleotide) includes at least two barcode sequences (e.g., BCb1 and BCb2). In some instances, the second probe oligonucleotide (e.g., the second polynucleotide) includes at least three, at least four, at least five, or more barcode sequences. The barcodes enable transcriptome-level multiplexing potential by methods of sequential hybridization of oligonucleotides that have a detectable moiety to one or more barcode sequence.

In some instances, the second probe oligonucleotide (e.g., the second polynucleotide) includes a third docking sequence (e.g., DR2) that is a docking site for hybridization of a third oligonucleotide (e.g., a splint). In some instances, the third docking sequence (e.g., DR2) is fully complementary to a sequence (e.g., a second region) of a third probe oligonucleotide (e.g., a splint). In some instances, the third docking sequence (e.g., DR2) is partially (e.g., at least 70%, at least 75%, at least 80%, at least 85%, at least 90%, at least 91%, at least 92%, at least 93%, at least 94%, at least 95%, at least 96%, at least 97%, at least 98%, or at least 99%) complementary to a sequence (e.g., a second region) of a third probe oligonucleotide (e.g., a splint).

In some instances, the second probe oligonucleotide (e.g., the second polynucleotide) includes a fourth docking sequence (e.g., DR2′) that is another docking site for hybridization of a third probe oligonucleotide (e.g., a splint) to the second probe oligonucleotide (e.g., the second polynucleotide). In some instances, the third docking sequence (e.g., DR2) and the fourth docking sequence (e.g., DR2′) are at opposite (e.g., 5′ or 3′) ends of the second probe oligonucleotide (e.g., the second polynucleotide).

In some instances, the second probe oligonucleotide (e.g., the second polynucleotide) includes, in order from 5′ to 3′: a third docking sequence (e.g., DR2), a sequence that is complementary to a sequence of an analyte (e.g., HR2 complementary to HR2′ of a target nucleic acid), one or more barcode sequences (e.g., BCb1, . . . , and BCbj), and a fourth docking sequence (e.g., DR2′). In some instances, the second probe oligonucleotide (e.g., the second polynucleotide) includes, in order from 5′ to 3′: a fourth docking sequence (e.g., DR2′), one or more barcode sequences (e.g., BCb1, . . . , and BCbj), a sequence that is complementary to a sequence of an analyte e.g., HR2 complementary to HR2′ of a target nucleic acid), and a third docking sequence (e.g., DR2).

(iii) Third Probe Oligonucleotide (e.g., Splint)

Also disclosed herein is a third probe oligonucleotide (e.g., a splint). In some embodiments, the third probe oligonucleotide (e.g., the splint) includes sequences that are fully or partially complementary to two sequences of the first probe oligonucleotide (e.g., the first polynucleotide) and to two sequences of the second probe oligonucleotide (e.g., the second polynucleotide). In some instances, the third probe oligonucleotide (e.g., the splint) includes sequences that are fully or partially complementary to a fragment on the 5′ end of the first probe oligonucleotide (e.g., the first polynucleotide), a fragment on the 3′ end of the first oligonucleotide (e.g., the first polynucleotide), a fragment on the 5′ end of the second probe oligonucleotide (e.g., the second polynucleotide), and a fragment on the 3′ end of the second probe oligonucleotide (e.g., the second polynucleotide).

In some embodiments, the splint is used to bring various regions of the first polynucleotide in proximity. In some embodiments, the splint is used to bring various regions of the second polynucleotide in proximity. In some embodiments, the splint is used to bring the first polynucleotide in proximity with the second polynucleotide. In some cases, the splint is used to bring two sequences in sufficient proximity for hybridization and/or ligation. In some embodiments, the splint may also serve as a primer for an amplification reaction (e.g., RCA of the circular polynucleotide can then be initiated using the splint polynucleotide as a primer), and in some instances a separate primer may be provided for the amplification reaction. In some instances, each splint molecule can be used to facilitate two or more separate ligations. For example, the splint may span two or more different junctions where the ligations can occur. In some cases, one splint molecule can be used to facilitate the ligation of DR1 to DR2 and ligation of DR1′ to DR2′, which may be catalyzed by the same ligase or by different ligases and may occur simultaneously or sequentially in any order.

In some instances, the third probe oligonucleotide (e.g., the splint) includes sequences (e.g., sequences contained in a second region of the splint) that are fully or partially complementary to the first docking sequence (e.g., DR1) on the first probe oligonucleotide (e.g., the first polynucleotide). In some instances, the third probe oligonucleotide (e.g., the splint) includes sequences (e.g., sequences contained in a first region of the splint) that are fully or partially complementary to the second docking sequence (e.g., DR1′) on the first probe oligonucleotide (e.g., the first polynucleotide). In some instances, the third probe oligonucleotide (e.g., the splint) includes sequences (e.g., sequences contained in the second region of the splint) that are fully or partially complementary to the third docking sequence (e.g., DR2) on the second probe oligonucleotide (e.g., the second polynucleotide). In some instances, the third probe oligonucleotide (e.g., the splint) includes sequences (e.g., sequences contained in the first region of the splint) that are fully or partially complementary to the fourth docking sequence (e.g., DR2′) on the second probe oligonucleotide (e.g., the second polynucleotide).

In some instances, the third probe oligonucleotide (e.g., the splint) hybridizes to the first probe oligonucleotide (e.g., the first polynucleotide) and the second probe oligonucleotide (e.g., the second polynucleotide) at the second and fourth docking sequences (e.g., DR1′ and DR2′), respectively. That is, in some instances, the third probe oligonucleotide (e.g., the splint) hybridizes to the regions of the first probe oligonucleotide (e.g., the first polynucleotide) and the second probe oligonucleotide (e.g., the second polynucleotide) that are opposite of the sequence that hybridizes to the analyte (e.g., that are opposite HR1 and HR2 that hybridize to the target nucleic acid). Thus, in some instances, the third probe oligonucleotide (e.g., the splint) has two sequences of hybridization (instead of four sequences) to the first and second oligonucleotide (e.g., as shown in FIG. 4).

The 5′ and 3′-ends of the docking regions of the first and second polynucleotides may lie directly adjacent to one another or there may be a nick or gap between the ends that may require gap filling, using the splint as a template. In some embodiments, there are nicks between DR1 and DR1′ and between DR2 and DR2′. In some embodiments, there are gaps between DR1 and DR1′ and between DR2 and DR2′. In some embodiments, one of DR1-DR1′ and DR2-DR2′ is separated by a nick while other is separated by a gap. In some embodiments, the gap between DR1 and DR2 and the gap between DR1′ and DR2′ are, independent of each other, are between 1 and 10 nucleotides in length. In some embodiments, the gap between DR1 and DR2 and the gap between DR1′ and DR2′ are, independent of each other, are one, two, three, four, five, six, seven, eight, nine, 10, or more than 10 nucleotides in length. In an embodiment comprising a plurality of polynucleotides, both types of junctions may be present and gaps of different lengths can also be present.

In some of any such embodiments, HR1 of the first polynucleotide hybridizes to the target nucleic acid and/or HR2 of the second polynucleotide hybridizes to the target nucleic acid. In some embodiments, for example, in FIG. 2 and FIG. 4, hybridization of the first polynucleotide to the target nucleic acid (e.g., HR1 to HR1′) occurs simultaneously with, prior to, or after hybridization of the second polynucleotide to the target nucleic acid (e.g., HR2 to HR2′). In some aspects, ligation of DR1′ to DR2′ and/or DR1 to DR2 does not occur unless HR1 of the first polynucleotide and/or HR2 of the second polynucleotide is hybridized to the target nucleic acid. In some aspects, hybridization of HR1 of the first polynucleotide and/or HR2 of the second polynucleotide to the target nucleic acid provides conditions sufficient (e.g., stability) for downstream events such as hybridization of DR1′ to DR1; hybridization of DR2′ to DR2; hybridization of DR1′, DR1, DR2′, DR2 to a region of the splint; and/or ligation of DR1′ to DR2′ and/or DR1 to DR2. In some embodiments, the length of the regions DR1, DR2, DR1′, DR2′, HR1, and HR2, can be tuned accordingly for the desired stability of hybridization conditions of complementary regions.

In some embodiments, each of the two polynucleotides is first hybridized to the target nucleic acid at a temperature higher than the melting temperatures of splint/docking region hybridization. After removing unhybridized and/or nonspecifically hybridized polynucleotides (e.g., through a stringent wash), the temperature is lowered to about the melting temperature of docking region hybridization, e.g., within about 5, 4, 3, 2, or 1 degree above or below the melting temperature of docking region hybridization, and the splint is provided in order to bring into close proximity with one another the free ends of DR1 and DR2 and the free ends of DR1′ and DR2′. The free ends of DR1 and DR2, as well as the free ends of DR1′ and DR2′, can then be ligated to one another either with or without gap filling, thus forming a circular polynucleotide hybridized to the target nucleic acid. RCA of the circular polynucleotide can then be initiated, e.g., using the splint polynucleotide as a primer.

In some embodiments, each of the two polynucleotides is first hybridized to the target nucleic acid at a temperature higher than the melting temperatures of splint/docking region hybridization. After removing unhybridized and/or nonspecifically hybridized polynucleotides (e.g., through a stringent wash), the temperature is lowered to below the melting temperature of splint/docking region hybridization, and the splint is provided in order to bring into close proximity with one another the free ends of DR1 and DR2 and the free ends of DR1′ and DR2′. The free ends of DR1 and DR2, as well as the free ends of DR1′ and DR2′, can then be ligated to one another either with or without gap filling, thus forming a circular polynucleotide hybridized to the target nucleic acid. RCA of the circular polynucleotide can then be initiated, e.g., using the splint polynucleotide as a primer.

In some embodiments, a target nucleic acid is contacted with each of the two polynucleotides and the splint, at a temperature higher than the melting temperatures of splint/docking region hybridization. At this temperature, the two polynucleotides hybridize to the target nucleic acid, while there is little or no splint/docking region hybridization. The temperature can then be lowered to below the melting temperature of splint/docking region hybridization, such that the splint brings into close proximity with one another the free ends of DR1 and DR2 and the free ends of DR1′ and DR2′. Unhybridized and/or nonspecifically hybridized polynucleotides may be removed, e.g., through a stringent wash. The free ends of DR1 and DR2, as well as the free ends of DR1′ and DR2′, can then be ligated to one another either with or without gap filling, thus forming a circular polynucleotide hybridized to the target nucleic acid. RCA of the circular polynucleotide can then be initiated, e.g., using the splint polynucleotide as a primer.

In some embodiments, a target nucleic acid is contacted with each of the two polynucleotides and the splint, at a temperature lower than the melting temperatures of splint/docking region hybridization. At this temperature, the two polynucleotides hybridize to the target nucleic acid. In addition, the docking regions also hybridize to the splint, bringing into close proximity with one another the free ends of DR1 and DR2 and the free ends of DR1′ and DR2′. Unhybridized and/or nonspecifically hybridized polynucleotides may be removed, e.g., through a stringent wash. The free ends of DR1 and DR2, as well as the free ends of DR1′ and DR2′, can then be ligated to one another either with or without gap filling, thus forming a circular polynucleotide hybridized to the target nucleic acid. RCA of the circular polynucleotide can then be initiated, e.g., using the splint polynucleotide as a primer.

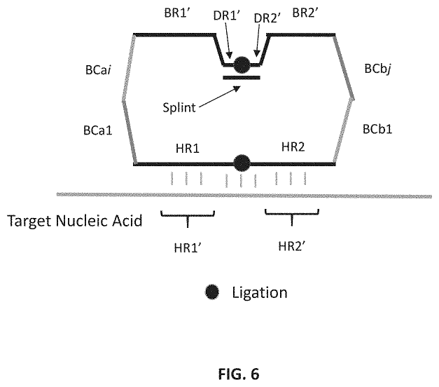

FIG. 6 shows an alternative embodiment. In this embodiment, a splint and the target nucleic acid of interest are utilized to facilitate the ligation of the polynucleotides. The arrangement of the various components is similar to the arrangement shown in FIG. 2, however there are no docking regions DR1 or DR2. Instead, HR1 and HR2 hybridize to the target nucleic acid of interest and a nick or gap is formed between HR1 and HR2, while DR1′ and DR2′ are complementary to regions of the splint. In some embodiments, the hybridization of HR1 and HR2 to the target nucleic acid of interest provides the 5′ and 3′-ends required for a ligation reaction. In some embodiments, this arrangement is a DNA/RNA complex, and a ligase having an DNA-RNA templated ligase activity catalyzes the ligation. In some embodiments, the complex formed by DR1′, DR2′ and the splint supplies the 5′ and 3′-ends for a ligation reaction, however this reaction is templated by the splint and catalyzed by a ligase having a DNA-DNA templated ligase activity, such as a T4 DNA ligase. The arrangements of features shown in FIG. 6 may be 5′ to 3′ or 3′ to 5′. For instance, each docking region DR1′ or DR2′ can be a 3′ or 5′ end sequence that is complementary to a portion of the splint, and each hybridization region HR1 or HR2 can be a 3′ or 5′ end sequence that is complementary to a portion of the target nucleic acid, e.g., an RNA such as an mRNA molecule.

As an alternative to the embodiment in FIG. 6, in some instances there are no docking regions DR1′ or DR2′. Instead, there are docking regions DR1 and DR2 and bridge regions BR1 and BR2; while HR1 and HR2 hybridize to the target nucleic acid of interest (to HR1′ and HR2′, respectively) and DR1 and DR2 (instead of DR1′ and DR2′) hybridize to the splint, DR1′ and DR2′ hybridize to the target nucleic acid of interest, e.g., to the region between HR1′ and HR2′. Ligation between DR1 and DR2 can be a DNA-templated ligation while ligation between DR1′ and DR2′ can be an RNA-templated ligation.

In some embodiments, each of the two polynucleotides is first hybridized to the target nucleic acid at a temperature higher than the melting temperatures of splint/docking region hybridization. The target nucleic acid brings into close proximity with one another the free ends of HR1 and HR2. After removing unhybridized and/or nonspecifically hybridized polynucleotides (e.g., through a stringent wash), the temperature is lowered to about the melting temperature of splint/docking region hybridization, e.g., within about 5, 4, 3, 2, or 1 degree above or below the melting temperature of splint/docking region hybridization, and the splint is provided in order to bring into close proximity with one another the free ends of DR1′ and DR2′. The free ends of HR1 and HR2, as well as the free ends of DR1′ and DR2′, can be ligated to one another either with or without gap filling, thus forming a circular polynucleotide hybridized to the target nucleic acid. RCA of the circular polynucleotide can then be initiated using the splint polynucleotide as a primer.

In some embodiments, each of the two polynucleotides is first hybridized to the target nucleic acid at a temperature higher than the melting temperatures of splint/docking region hybridization. The target nucleic acid brings into close proximity with one another the free ends of HR1 and HR2. After removing unhybridized and/or nonspecifically hybridized polynucleotides (e.g., through a stringent wash), the temperature is lowered to below the melting temperature of splint/docking region hybridization, and the splint is provided in order to bring into close proximity with one another the free ends of DR1′ and DR2′. The free ends of HR1 and HR2, as well as the free ends of DR1′ and DR2′, can be ligated to one another either with or without gap filling, thus forming a circular polynucleotide hybridized to the target nucleic acid. RCA of the circular polynucleotide can then be initiated using the splint polynucleotide as a primer.

In some embodiments, a target nucleic acid is contacted with each of the two polynucleotides and the splint, at a temperature higher than the melting temperatures of splint/docking region hybridization. At this temperature, the two polynucleotides hybridize to the target nucleic acid, bringing into close proximity with one another the free ends of HR1 and HR2, while there is little or no splint/docking region hybridization. The temperature can then be lowered to below the melting temperature of splint/docking region hybridization, such that the splint brings into close proximity with one another the free ends of DR1′ and DR2′. Unhybridized and/or nonspecifically hybridized polynucleotides may be removed, e.g., through a stringent wash. The free ends of HR1 and HR2, as well as the free ends of DR1′ and DR2′, can be ligated to one another either with or without gap filling, thus forming a circular polynucleotide hybridized to the target nucleic acid. RCA of the circular polynucleotide can then be initiated, e.g., using the splint polynucleotide as a primer.

In some embodiments, a target nucleic acid is contacted with each of the two polynucleotides and the splint, at a temperature lower than the melting temperatures of splint/docking region hybridization. At this temperature, the two polynucleotides hybridize to the target nucleic acid, bringing into close proximity with one another the free ends of HR1 and HR2. In addition, the docking regions also hybridize to the splint, bringing into close proximity with one another the free ends of DR1′ and DR2′. Unhybridized and/or nonspecifically hybridized polynucleotides may be removed, e.g., through a stringent wash. The free ends of HR1 and HR2, as well as the free ends of DR1′ and DR2′, can be ligated to one another either with or without gap filling, thus forming a circular polynucleotide hybridized to the target nucleic acid. RCA of the circular polynucleotide can then be initiated, e.g., using the splint polynucleotide as a primer.

In some embodiments, the ligation of HR1 and HR2 (with or without prior gap filling) and the ligation of DR1′ and DR2′ (with or without prior gap filling) are catalyzed by two different kinds of ligases. In some embodiment, the ligation of HR1 and HR2 comprises an RNA-templated DNA ligation, and the ligation of DR1′ and DR2′ comprises a DNA-templated DNA ligation. In some embodiments, the first polynucleotide and/or the second polynucleotide do not comprise ribonucleotide(s) at or near a ligation site, e.g., at or near the 5′ or 3′ end of HR1, at or near the 5′ or 3′ end of HR2, at or near the 5′ or 3′ end of DR1′, and/or at or near the 5′ or 3′ end of DR2′. In some embodiments, the splint (e.g., in FIGS. 3-4 and 6) does not comprise ribonucleotide(s). In some embodiments, the first polynucleotide, the second polynucleotide, and/or the splint (e.g., in FIGS. 3-4 and 6) do not comprise an additional sequence 5′ to a hybridization region that forms a 5′ flap containing one or more nucleotides at its 3′ end that is cleaved prior to ligation (e.g., using a 5′ Flap endonuclease (FEN) or other suitable enzyme with 5′ exonuclease activity).

Although the drawings discussed above illustrate the assembly of two polynucleotides to form a padlock or circular probe, a plurality of polynucleotides may be assembled utilizing the structural components described. As such, padlock or circular probes comprising 2, 3, 4, 5, 6, 7, 8, 9, 10, or more polynucleotides are contemplated.

The docking region disclosed here serve to facilitate the binding of the polynucleotide to a cognate sequence so that a ligation reaction can be catalyzed. In one embodiment a polynucleotide will comprise one or more docking regions. The number of docking regions can be related to the number of ligation events occurring to form the circular probe. For example, when two polynucleotides are ligated to form a circular probe, four docking regions may be present, when three polynucleotides are ligated to form a circular probe, six docking regions may be present, and so on. In FIG. 6, the docking regions can be viewed as comprising the portion of the polynucleotides that hybridize with the target nucleic acid of interest. Docking regions can be between 1 and about 20 nucleotides in length. In some embodiments, the docking regions described herein can be, independent of each other, about 2, about 4, about 6, about 8, about 10, about 12, about 14, about 16, about 18, or more than 18 nucleotides in length.

The sequence composition and length of the docking regions may be selected by considering the melting temperature (Tₘ) of the desired interaction of the docking regions, if the polynucleotide constituents directly interact with the docking regions of another polynucleotide, or with a splint probe. In some embodiments, where a plurality of hybridization regions are present, the melting temperature (Tₘ) of each sequence (polynucleotide and target nucleic acid of interest) is substantially the same. In other embodiments, the temperatures of melting are between about 40° C. and about 70° C. In some embodiments, the docking region temperatures of melting may be lower than or similar to room temperature, e.g., between about 16° C. and about 40° C.

The padlock or circular probes comprising of a plurality of polynucleotides will comprise at least one barcode sequence. In one embodiment, where a plurality of polynucleotides are assembled into a circular probe, one polynucleotide may comprise one or more barcode sequences, while the other polynucleotide or polynucleotides either lack a barcode sequence or contain one or more barcode sequences.

In some embodiments, more than two polynucleotides can be assembled to form a padlock or circular probe as disclosed herein. For example, after formation of a padlock or circular probe between a first polynucleotide and a second polynucleotide, the padlock or circular probe may optionally be cleaved in order to assemble a third polynucleotide. In one embodiment, a first polynucleotide comprises barcode sequence BCa1 and optionally additional barcode sequence(s) BCai, wherein i is an integer of 1 or greater, a second polynucleotide comprises barcode sequence BCb1, and optionally additional barcode sequence(s) BCbj, wherein j is an integer of 1 or greater, a third polynucleotide comprises barcode sequences BCc1 and optionally additional barcode sequence(s) BCck, wherein k is an integer of 1 or greater, etc., and the values of i, j, and k can be selected independent of each other. In one embodiment, the barcode sequences are distinct and do not overlap one another. In another embodiment, the barcode sequences overlap.

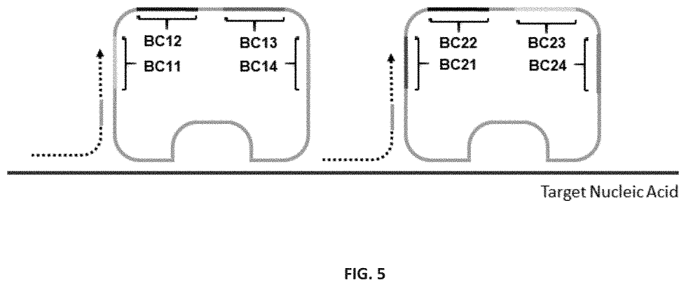

In some aspects of the present disclosure, target nucleic acids are targeted through primary probes, which are barcoded through the incorporation of specific sequences into the primary probes, in addition to the sequence that binds the targeted nucleic acid. In some embodiments, a targeting probe (e.g., a primary probe that binds directly to an RNA molecule) comprises a plurality of barcodes, such as 2, 3, 4, 5, 6, 7, 8, 9, 10, or more barcodes. The barcodes may comprise overlapping or non-overlapping sequences. In some embodiments, a primary probe having a plurality of barcodes, such as two, three, four, five, six, seven, eight, or more barcodes, may bypass the need of using secondary probes having secondary barcodes. For example, as shown in FIG. 5, different barcode subsets BC11/BC12/BC13/BC14 and BC21/BC22/BC23/BC24 can be allocated on different probe subsets. In some embodiments, a primary probe having a plurality of barcodes may be constructed using a plurality of polynucleotides disclosed herein, e.g., as shown in any of in FIGS. 1-4 and 6.

Heterogeneous assemblies of polynucleotides are contemplated as components of the circular probes disclosed here. For example, a circular probe can comprise first and second polynucleotides and disposed between them a plurality of intervening sequences comprising other functional components such as one or more barcode sequences.

Although the barcode sequences described herein can be any suitable length, barcoded sequences are typically between about 5 and about 30 nucleotides in length, e.g., between about 10 and about 25 nucleotides in length, and can serve as is a unique identifier of a gene, is an error-checking barcode, and/or identifies an mRNA as a splice variant and/or identify a splice junction sequence, as non-limiting examples.

Composite padlock or circular probes comprising of a plurality of polynucleotides may comprise at least one bridge region. In one embodiment, where a plurality of polynucleotides are assembled into a circular probe, one polynucleotide may comprise a plurality of bridge regions while the other polynucleotides comprising the circular probe either lack a bridge region or contain one or more bridge region. Bridge regions are between about 1 and about 20 nucleotides in length. In some embodiments, the bridge regions described herein can be, independent of each other, about 2, about 4, about 6, about 8, about 10, about 12, about 14, about 16, about 18, or more than 18 nucleotides in length.

Composite padlock or circular probes comprising a plurality of polynucleotides will comprise at least one hybridization region per assembled circular probe. In another embodiment, where two polynucleotides are ligated to form the circular probe, at least one hybridization region is present. Although the hybridization sequences described herein can be any suitable length, hybridization regions are preferably between about 5 and 35 nucleotides in length, preferably between about 8 and about 25 nucleotides in length, and preferably between about 10 and 20 nucleotides in length. In some embodiments, the hybridization regions are about 5, about 10, about 15, about 20, about 25, about 30, or about 35 nucleotides in length. In an embodiment containing multiple hybridization regions, those regions may be substantially identical in length or the length of these regions may differ.

The sequence composition and length of the hybridization regions is affected by the melting temperature (Tₘ) of the desired interaction of the hybridization region of the polynucleotide and the corresponding cognate region in the target nucleic acid of interest, e.g., the hybridization complex. In some embodiments, where a plurality of hybridization regions are present, the Tₘ of each sequence (polynucleotide and target nucleic acid of interest) is substantially the same. In other embodiments, the temperatures of melting are between about 40° C. and about 70° C. In some embodiments, the Tₘ of the docking regions may be lower than the Tₘ of the hybridization regions in the hybridization complex.

In some embodiments, the hybridization complex is formed at a temperature between about 30° C. and about 50° C., e.g., about 40° C. In any of the preceding embodiments, the hybridization complex can be formed at a temperature between about 16° C. and about 40° C. In some embodiments, the hybridization complex is formed at a temperature between or between about 10° C. and about 30° C., e.g., about 16° C. In some embodiments, the hybridization complex is formed at or at about 16° C.

In any of the embodiments disclosed herein, each of the plurality of polynucleotides forming the composite padlock or circular probes, and any splint disclosed herein, can be any suitable length independent of one another, for example, between about 5 and about 10, between about 10 and about 20, between about 20 and about 25, between about 25 and about 30, between about 30 and about 35, between about 35 and about 40, between about 40 and about 45, between about 45 and about 50, between about 50 and about 60, between about 60 and about 70, between about 70 and about 80, between about 80 and about 90, between about 90 and about 100, between about 100 and about 110, between about 110 and about 120, between about 120 and about 130, between about 130 and about 140, between about 140 and about 150, between about 150 and about 160, between about 160 and about 170, between about 170 and about 180, between about 180 and about 190, or between about 190 and about 200 nucleotides in length.

In any of the embodiments disclosed herein, the circular probe formed by the plurality of polynucleotides can be any suitable length, for example, between about 20 and about 25, between about 25 and about 30, between about 30 and about 35, between about 35 and about 40, between about 40 and about 45, between about 45 and about 50, between about 50 and about 60, between about 60 and about 70, between about 70 and about 80, between about 80 and about 90, between about 90 and about 100, between about 100 and about 110, between about 110 and about 120, between about 120 and about 130, between about 130 and about 140, between about 140 and about 150, between about 150 and about 160, between about 160 and about 170, between about 170 and about 180, between about 180 and about 190, between about 190 and about 200, between about 200 and about 250, between about 250 and about 300, between about 300 and about 350, between about 350 and about 400, or more than 400 nucleotides in length.

In any of the embodiments disclosed herein, each of the plurality of polynucleotides forming the composite padlock or circular probes, and any splint disclosed herein, can be a modified nucleic acid molecule or comprise modified nucleotides or modified nucleosides, such as methylated nucleotides and nucleotide analogs, uracyl, other sugars, and linking groups such as fluororibose and thioate, and nucleotide branches. In some embodiments, the polynucleotide and/or the splint may include non-nucleotide components. Exemplary modified nucleic acids include amine-modified nucleotides such as aminoallyl (aa)-dUTP, aa-dCTP, aa-dGTP, and/or aa-dATP, 2-Aminopurine, 2,6-Diaminopurine (2-Amino-dA), inverted dT, 5-Methyl dC, 2′-deoxy-Inosine, Super T (5-hydroxybutynl-2′-deoxyuridine), Super G (8-aza-7-deazaguanosine), locked nucleic acids (LNAs), unlocked nucleic acids (UNAs, e.g., UNA-A, UNA-U, UNA-C, UNA-G), Iso-dG, Iso-dC, 2′ Fluoro bases (e.g., Fluoro C, Fluoro U, Fluoro A, and Fluoro G), and combinations of the foregoing.

In any of the embodiments disclosed herein, each of the plurality of polynucleotides forming the composite padlock or circular probes, and any splint disclosed herein, can be a DNA molecule comprising one or more ribonucleotides.

### II. Samples, Analytes, and Target Nucleic Acids

A target nucleic acid in a sample that may be processed and/or analyzed using a method disclosed herein may be or be comprised in an analyte (e.g., a nucleic acid analyte, such as genomic DNA, mRNA transcript, or cDNA, or a product thereof, e.g., an extension or amplification product, such as an RCA product) and/or may be or be comprised in a labelling agent for one or more analytes (e.g., a nucleic acid analyte or a non-nucleic acid analyte) in a sample. Exemplary analytes and labelling agents are described below.

A. Samples

A sample disclosed herein can be or derived from any biological sample. Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. In addition to the subjects described above, a biological sample can be obtained from a prokaryote such as a bacterium, an archaea, a virus, or a viroid. A biological sample can also be obtained from non-mammalian organisms (e.g., a plant, an insect, an arachnid, a nematode, a fungus, or an amphibian). A biological sample can also be obtained from a eukaryote, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

The biological sample can include any number of macromolecules, for example, cellular macromolecules and organelles (e.g., mitochondria and nuclei). The biological sample can be a nucleic acid sample and/or protein sample. The biological sample can be a carbohydrate sample or a lipid sample. The biological sample can be obtained as a tissue sample, such as a tissue section, biopsy, a core biopsy, needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample may comprise cells which are deposited on a surface.

Cell-free biological samples can include extracellular polynucleotides. Extracellular polynucleotides can be isolated from a bodily sample, e.g., blood, plasma, serum, urine, saliva, mucosal excretions, sputum, stool, and tears.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

Biological samples can include analytes (e.g., protein, RNA, and/or DNA) that are embedded in a matrix (e.g., in a 3D matrix). In some embodiments, amplicons (e.g., rolling circle amplification products) derived from or associated with analytes (e.g., protein, RNA, and/or DNA) can be embedded in a 3D matrix. In some embodiments, a 3D matrix may comprise a network of natural molecules and/or synthetic molecules that are chemically and/or enzymatically linked, e.g., by crosslinking. In some embodiments, a 3D matrix may comprise a synthetic polymer. In some embodiments, a 3D matrix comprises a hydrogel.

In some embodiments, a substrate herein can be any support that is insoluble in aqueous liquid and which allows for positioning of biological samples, analytes, features, and/or reagents (e.g., probes) on the support. In some embodiments, a biological sample can be attached to a substrate. Attachment of the biological sample can be irreversible or reversible, depending upon the nature of the sample and subsequent steps in the analytical method. In certain embodiments, the sample can be attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose.

In some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

### (i) Tissue Sectioning

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick.

More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analysed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analysed successively to obtain three-dimensional information about the biological sample.

### (ii) Freezing

In some embodiments, the biological sample (e.g., a tissue section as described above) can be prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

(iii) Fixation and Postfixation

In some embodiments, the biological sample can be prepared using formalin-fixation and paraffin-embedding (FFPE), which are established methods. In some embodiments, cell suspensions and other non-tissue samples can be prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, acetone fixation is used with fresh frozen samples, which can include, but are not limited to, cortex tissue, mouse olfactory bulb, human brain tumor, human post-mortem brain, and breast cancer samples. When acetone fixation is performed, pre-permeabilization steps (described below) may not be performed. Alternatively, acetone fixation can be performed in conjunction with permeabilization steps.

In some embodiments, the methods provided herein comprises one or more post-fixing (also referred to as postfixation) steps. In some embodiments, one or more post-fixing step is performed after contacting a sample with a polynucleotide disclosed herein, e.g., one or more probes such as a circular or padlock probe. In some embodiments, one or more post-fixing step is performed after a hybridization complex comprising a probe and a target is formed in a sample. In some embodiments, one or more post-fixing step is performed prior to a ligation reaction disclosed herein, such as the ligation to circularize a padlock probe.

In some embodiments, one or more post-fixing step is performed after contacting a sample with a binding or labelling agent (e.g., an antibody or antigen binding fragment thereof) for a non-nucleic acid analyte such as a protein analyte. The labelling agent can comprise a nucleic acid molecule (e.g., reporter oligonucleotide) comprising a sequence corresponding to the labelling agent and therefore corresponds to (e.g., uniquely identifies) the analyte. In some embodiments, the labelling agent can comprise a reporter oligonucleotide comprising one or more barcode sequences.

A post-fixing step may be performed using any suitable fixation reagent disclosed herein, for example, 3% (w/v) paraformaldehyde in DEPC-PBS.

### (iv) Embedding

As an alternative to paraffin embedding described above, a biological sample can be embedded in any of a variety of other embedding materials to provide structural substrate to the sample prior to sectioning and other handling steps. In some cases, the embedding material can be remove, e.g., prior to analysis of tissue sections obtained from the sample. Suitable embedding materials include, but are not limited to, waxes, resins (e.g., methacrylate resins), epoxies, and agar.

In some embodiments, the biological sample can be embedded in a matrix (e.g., a hydrogel matrix). In some aspects, the embedding material can be applied to the sample one or more times. Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

In some embodiments, the biological sample is immobilized in the hydrogel via cross-linking of the polymer material that forms the hydrogel. Cross-linking can be performed chemically and/or photochemically, or alternatively by any other hydrogel-formation method known in the art.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 μm to about 2 mm.

Additional methods and aspects of hydrogel embedding of biological samples are described for example in Chen et al., Science 347(6221):543-548, 2015, the entire contents of which are incorporated herein by reference.

### (v) Staining

To facilitate visualization, biological samples can be stained using a wide variety of stains and staining techniques. In some embodiments, for example, a sample can be stained using any number of stains, including but not limited to, acridine orange, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, propidium iodide, rhodamine, or safranine.

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and Giemsa stain.

In some embodiments, biological samples can be destained. Methods of destaining or discoloring a biological sample are known in the art, and generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

### (vi) Isometric Expansion

In some embodiments, a biological sample embedded in a matrix (e.g., a hydrogel) can be isometrically expanded. Isometric expansion methods that can be used include hydration, a preparative step in expansion microscopy, as described in Chen et al., Science 347(6221):543-548, 2015.

Isometric expansion can be performed by anchoring one or more components of a biological sample to a gel, followed by gel formation, proteolysis, and swelling. In some embodiments, analytes in the sample, products of the analytes, and/or probes associated with analytes in the sample can be anchored to the matrix (e.g., hydrogel). Isometric expansion of the biological sample can occur prior to immobilization of the biological sample on a substrate, or after the biological sample is immobilized to a substrate. In some embodiments, the isometrically expanded biological sample can be removed from the substrate prior to contacting the substrate with probes disclosed herein.

In general, the steps used to perform isometric expansion of the biological sample can depend on the characteristics of the sample (e.g., thickness of tissue section, fixation, cross-linking), and/or the analyte of interest (e.g., different conditions to anchor RNA, DNA, and protein to a gel).

In some embodiments, proteins in the biological sample are anchored to a swellable gel such as a polyelectrolyte gel. An antibody can be directed to the protein before, after, or in conjunction with being anchored to the swellable gel. DNA and/or RNA in a biological sample can also be anchored to the swellable gel via a suitable linker. Examples of such linkers include, but are not limited to, 6-((Acryloyl)amino) hexanoic acid (Acryloyl-X SE) (available from ThermoFisher, Waltham, Mass.), Label-IT Amine (available from MirusBio, Madison, Wis.) and Label X (described for example in Chen et al., Nat. Methods 13:679-684, 2016, the entire contents of which are incorporated herein by reference).

Isometric expansion of the sample can increase the spatial resolution of the subsequent analysis of the sample. The increased resolution in spatial profiling can be determined by comparison of an isometrically expanded sample with a sample that has not been isometrically expanded.

In some embodiments, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some embodiments, the sample is isometrically expanded to at least 2× and less than 20× of its non-expanded size.

(vii) Crosslinking and De-Crosslinking

In some embodiments, the biological sample is reversibly cross-linked prior to or during an in situ assay round. In some aspects, the analytes, polynucleotides and/or amplification product (e.g., amplicon) of an analyte or a probe (e.g., first and/or second polynucleotide) bound thereto can be anchored to a polymer matrix. For example, the polymer matrix can be a hydrogel. In some embodiments, one or more of the polynucleotide probe(s) and/or amplification product (e.g., amplicon) thereof can be modified to contain functional groups that can be used as an anchoring site to attach the polynucleotide probes and/or amplification product to a polymer matrix. In some embodiments, a modified probe comprising oligo dT may be used to bind to mRNA molecules of interest, followed by reversible crosslinking of the mRNA molecules.

In some embodiments, the biological sample is immobilized in a hydrogel via cross-linking of the polymer material that forms the hydrogel. Cross-linking can be performed chemically and/or photochemically, or alternatively by any other hydrogel-formation method known in the art. A hydrogel may include a macromolecular polymer gel including a network. Within the network, some polymer chains can optionally be cross-linked, although cross-linking does not always occur.

In some embodiments, a hydrogel can include hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some embodiments, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference.

In some embodiments, the hydrogel can form the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

In some embodiments, hydrogel formation on a substrate occurs before, contemporaneously with, or after probes are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

In embodiments in which a hydrogel is formed within a biological sample, functionalization chemistry can be used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible.

In some embodiments, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, transposase, ligase, proteinase K, and DNAse. Additional reagents can also include reverse transcriptase enzymes, including enzymes with terminal transferase activity, primers, and switch oligonucleotides. In some embodiments, optical labels are added to the hydrogel subunits before, contemporaneously with, and/or after polymerization.

In some embodiments, HTC reagents are added to the hydrogel before, contemporaneously with, and/or after polymerization. In some embodiments, a cell labelling agent is added to the hydrogel before, contemporaneously with, and/or after polymerization. In some embodiments, a cell-penetrating agent is added to the hydrogel before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums).

In some embodiments, a method disclosed herein comprises de-crosslinking the reversibly cross-linked biological sample. The de-crosslinking does not need to be complete. In some embodiments, only a portion of crosslinked molecules in the reversibly cross-linked biological sample are de-crosslinked and allowed to migrate.

(viii) Tissue Permeabilization and Treatment

In some embodiments, a biological sample can be permeabilized to facilitate transfer of analytes out of the sample, and/or to facilitate transfer of species (such as probes) into the sample. If a sample is not permeabilized sufficiently, the amount of analyte captured from the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™ or Tween-20™), and enzymes (e.g., trypsin, proteases). In some embodiments, the biological sample can be incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some embodiments, the biological sample can be permeabilized by adding one or more lysis reagents to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes.

Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

In some embodiments, the biological sample can be permeabilized by non-chemical permeabilization methods. Non-chemical permeabilization methods are known in the art. For example, non-chemical permeabilization methods that can be used include, but are not limited to, physical lysis techniques such as electroporation, mechanical permeabilization methods (e.g., bead beating using a homogenizer and grinding balls to mechanically disrupt sample tissue structures), acoustic permeabilization (e.g., sonication), and thermal lysis techniques such as heating to induce thermal permeabilization of the sample.

Additional reagents can be added to a biological sample to perform various functions prior to analysis of the sample. In some embodiments, DNase and RNase inactivating agents or inhibitors such as proteinase K, and/or chelating agents such as EDTA, can be added to the sample. For example, a method disclosed herein may comprise a step for increasing accessibility of a nucleic acid for binding, e.g., a denaturation step to opening up DNA in a cell for hybridization by a probe. For example, proteinase K treatment may be used to free up DNA with proteins bound thereto.

### (ix) Selective Enrichment of RNA Species

In some embodiments, where RNA is the analyte, one or more RNA analyte species of interest can be selectively enriched. For example, one or more species of RNA of interest can be selected by addition of one or more oligonucleotides to the sample. In some embodiments, the additional oligonucleotide is a sequence used for priming a reaction by an enzyme (e.g., a polymerase). For example, one or more primer sequences with sequence complementarity to one or more RNAs of interest can be used to amplify the one or more RNAs of interest, thereby selectively enriching these RNAs.

In some embodiments, one or more nucleic acid probes can be used to hybridize to a target nucleic acid (e.g., cDNA or RNA molecule, such as an mRNA) and ligated in a templated ligation reaction (e.g., RNA-templated ligation (RTL) or DNA-templated ligation (e.g., on cDNA)) to generate a product for analysis. In some aspects, when two or more analytes are analyzed, a first and second probe that is specific for (e.g., specifically hybridizes to) each RNA or cDNA analyte are used. For example, in some embodiments of the methods provided herein, templated ligation is used to detect gene expression in a biological sample. An analyte of interest (such as a protein), bound by a labelling agent or binding agent (e.g., an antibody or epitope binding fragment thereof), wherein the binding agent is conjugated or otherwise associated with a reporter oligonucleotide comprising a reporter sequence that identifies the binding agent, can be targeted for analysis. Probes may be hybridized to the reporter oligonucleotide and ligated in a templated ligation reaction to generate a product for analysis. In some embodiments, gaps between the probe oligonucleotides may first be filled prior to ligation, using, for example, Mu polymerase, DNA polymerase, RNA polymerase, reverse transcriptase, VENT polymerase, Taq polymerase, and/or any combinations, derivatives, and variants (e.g., engineered mutants) thereof. In some embodiments, the assay can further include amplification of templated ligation products (e.g., by multiplex PCR).

In some embodiments, an oligonucleotide with sequence complementarity to the complementary strand of captured RNA (e.g., cDNA) can bind to the cDNA. For example, biotinylated oligonucleotides with sequence complementary to one or more cDNA of interest binds to the cDNA and can be selected using biotinylation-strepavidin affinity using any of a variety of methods known to the field (e.g., streptavidin beads).

Alternatively, one or more species of RNA can be down-selected (e.g., removed) using any of a variety of methods. For example, probes can be administered to a sample that selectively hybridize to ribosomal RNA (rRNA), thereby reducing the pool and concentration of rRNA in the sample. Additionally and alternatively, duplex-specific nuclease (DSN) treatment can remove rRNA (see, e.g., Archer, et al, Selective and flexible depletion of problematic sequences from RNA-seq libraries at the cDNA stage, BMC Genomics, 15 401, (2014), the entire contents of which are incorporated herein by reference). Furthermore, hydroxyapatite chromatography can remove abundant species (e.g., rRNA) (see, e.g., Vandernoot, V. A., cDNA normalization by hydroxyapatite chromatography to enrich transcriptome diversity in RNA-seq applications, Biotechniques, 53(6) 373-80, (2012), the entire contents of which are incorporated herein by reference).

A biological sample may comprise one or a plurality of analytes of interest. Methods for performing multiplexed assays to analyze two or more different analytes in a single biological sample are provided.

B. Analytes

The methods and compositions disclosed herein can be used to detect and analyze a wide variety of different analytes. In some aspects, an analyte can include any biological substance, structure, moiety, or component to be analyzed. In some aspects, a target disclosed herein may similarly include any analyte of interest. In some examples, a target or analyte can be directly or indirectly detected.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

The analyte may include any biomolecule, macromolecule, or chemical compound, including a protein or peptide, a lipid or a nucleic acid molecule, or a small molecule, including organic or inorganic molecules. The analyte may be a cell or a microorganism, including a virus, or a fragment or product thereof. An analyte can be any substance or entity for which a specific binding partner (e.g. an affinity binding partner) can be developed. Such a specific binding partner may be a nucleic acid probe (for a nucleic acid analyte) and may lead directly to the generation of a RCA template (e.g. a padlock or other circularizable probe). Alternatively, the specific binding partner may be coupled to a nucleic acid, which may be detected using an RCA strategy, e.g. in an assay which uses or generates a circular nucleic acid molecule which can be the RCA template.

Analytes of particular interest may include nucleic acid molecules, such as DNA (e.g. genomic DNA, mitochondrial DNA, plastid DNA, viral DNA, etc.) and RNA (e.g. mRNA, microRNA, rRNA, snRNA, viral RNA, etc.), and synthetic and/or modified nucleic acid molecules, (e.g. including nucleic acid domains comprising or consisting of synthetic or modified nucleotides such as LNA, PNA, morpholino, etc.), proteinaceous molecules such as peptides, polypeptides, proteins or prions or any molecule which includes a protein or polypeptide component, etc., or fragments thereof, or a lipid or carbohydrate molecule, or any molecule which comprise a lipid or carbohydrate component. The analyte may be a single molecule or a complex that contains two or more molecular subunits, e.g. including but not limited to protein-DNA complexes, which may or may not be covalently bound to one another, and which may be the same or different. Thus in addition to cells or microorganisms, such a complex analyte may also be a protein complex or protein interaction. Such a complex or interaction may thus be a homo- or hetero-multimer. Aggregates of molecules, e.g. proteins may also be target analytes, for example aggregates of the same protein or different proteins. The analyte may also be a complex between proteins or peptides and nucleic acid molecules such as DNA or RNA, e.g. interactions between proteins and nucleic acids, e.g. regulatory factors, such as transcription factors, and DNA or RNA.

(i) Endogenous Analytes

In some embodiments, an analyte herein is endogenous to a biological sample and can include nucleic acid analytes and non-nucleic acid analytes. Methods and compositions disclosed herein can be used to analyze nucleic acid analytes (e.g., using a nucleic acid probe or probe set that directly or indirectly hybridizes to a nucleic acid analyte) and/or non-nucleic acid analytes (e.g., using a labelling agent that comprises a reporter oligonucleotide and binds directly or indirectly to a non-nucleic acid analyte) in any suitable combination.

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte is inside a cell or on a cell surface, such as a transmembrane analyte or one that is attached to the cell membrane. In some embodiments, the analyte can be an organelle (e.g., nuclei or mitochondria). In some embodiments, the analyte is an extracellular analyte, such as a secreted analyte. Exemplary analytes include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

Examples of nucleic acid analytes include DNA analytes such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample.

Examples of nucleic acid analytes also include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

In some embodiments described herein, an analyte may be a denatured nucleic acid, wherein the resulting denatured nucleic acid is single-stranded. The nucleic acid may be denatured, for example, optionally using formamide, heat, or both formamide and heat. In some embodiments, the nucleic acid is not denatured for use in a method disclosed herein.

In certain embodiments, an analyte can be extracted from a live cell. Processing conditions can be adjusted to ensure that a biological sample remains live during analysis, and analytes are extracted from (or released from) live cells of the sample. Live cell-derived analytes can be obtained only once from the sample, or can be obtained at intervals from a sample that continues to remain in viable condition.

Methods and compositions disclosed herein can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate.

In any embodiment described herein, the analyte comprises a target sequence. In some embodiments, the target sequence may be endogenous to the sample, generated in the sample, added to the sample, or associated with an analyte in the sample. In some embodiments, the target sequence is a single-stranded target sequence (e.g., a sequence in a rolling circle amplification product). In some embodiments, the analytes comprise one or more single-stranded target sequences. In one aspect, a first single-stranded target sequence is not identical to a second single-stranded target sequence. In another aspect, a first single-stranded target sequence is identical to one or more second single-stranded target sequence. In some embodiments, the one or more second single-stranded target sequence is comprised in the same analyte (e.g., nucleic acid) as the first single-stranded target sequence. Alternatively, the one or more second single-stranded target sequence is comprised in a different analyte (e.g., nucleic acid) from the first single-stranded target sequence.

(ii) Labelling Agents

In some embodiments, provided herein are methods and compositions for analyzing endogenous analytes (e.g., RNA, ssDNA, and cell surface or intracellular proteins and/or metabolites) in a sample using one or more labelling agents. In some embodiments, an analyte labelling agent may include an agent that interacts with an analyte (e.g., an endogenous analyte in a sample). In some embodiments, the labelling agents can comprise a reporter oligonucleotide that is indicative of the analyte or portion thereof interacting with the labelling agent. For example, the reporter oligonucleotide may comprise a barcode sequence that permits identification of the labelling agent. In some cases, the sample contacted by the labelling agent can be further contacted with a probe (e.g., a single-stranded probe sequence), that hybridizes to a reporter oligonucleotide of the labelling agent, in order to identify the analyte associated with the labelling agent. In some embodiments, the analyte labelling agent comprises an analyte binding moiety and a labelling agent barcode domain comprising one or more barcode sequences, e.g., a barcode sequence that corresponds to the analyte binding moiety and/or the analyte. An analyte binding moiety barcode includes to a barcode that is associated with or otherwise identifies the analyte binding moiety. In some embodiments, by identifying an analyte binding moiety by identifying its associated analyte binding moiety barcode, the analyte to which the analyte binding moiety binds can also be identified. An analyte binding moiety barcode can be a nucleic acid sequence of a given length and/or sequence that is associated with the analyte binding moiety. An analyte binding moiety barcode can generally include any of the variety of aspects of barcodes described herein.

In some embodiments, the method comprises one or more post-fixing (also referred to as post-fixation) steps after contacting the sample with one or more labelling agents.

In the methods and systems described herein, one or more labelling agents capable of binding to or otherwise coupling to one or more features may be used to characterize analytes, cells and/or cell features. In some instances, cell features include cell surface features. Analytes may include, but are not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

In some embodiments, an analyte binding moiety may include any molecule or moiety capable of binding to an analyte (e.g., a biological analyte, e.g., a macromolecular constituent). A labelling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labelling agents can include (e.g., are attached to) a reporter oligonucleotide that is indicative of the cell surface feature to which the binding group binds. For example, the reporter oligonucleotide may comprise a barcode sequence that permits identification of the labelling agent. For example, a labelling agent that is specific to one type of cell feature (e.g., a first cell surface feature) may have coupled thereto a first reporter oligonucleotide, while a labelling agent that is specific to a different cell feature (e.g., a second cell surface feature) may have a different reporter oligonucleotide coupled thereto. For a description of exemplary labelling agents, reporter oligonucleotides, and methods of use, see, e.g., U.S. Pat. No. 10,550,429; U.S. Pat. Pub. 20190177800; and U.S. Pat. Pub. 20190367969, which are each incorporated by reference herein in their entirety.

In some embodiments, an analyte binding moiety includes one or more antibodies or antigen binding fragments thereof. The antibodies or antigen binding fragments including the analyte binding moiety can specifically bind to a target analyte. In some embodiments, the analyte is a protein (e.g., a protein on a surface of the biological sample (e.g., a cell) or an intracellular protein). In some embodiments, a plurality of analyte labelling agents comprising a plurality of analyte binding moieties bind a plurality of analytes present in a biological sample. In some embodiments, the plurality of analytes includes a single species of analyte (e.g., a single species of polypeptide). In some embodiments in which the plurality of analytes includes a single species of analyte, the analyte binding moieties of the plurality of analyte labelling agents are the same. In some embodiments in which the plurality of analytes includes a single species of analyte, the analyte binding moieties of the plurality of analyte labelling agents are the different (e.g., members of the plurality of analyte labelling agents can have two or more species of analyte binding moieties, wherein each of the two or more species of analyte binding moieties binds a single species of analyte, e.g., at different binding sites). In some embodiments, the plurality of analytes includes multiple different species of analyte (e.g., multiple different species of polypeptides).

In other instances, e.g., to facilitate sample multiplexing, a labelling agent that is specific to a particular cell feature may have a first plurality of the labelling agent (e.g., an antibody or lipophilic moiety) coupled to a first reporter oligonucleotide and a second plurality of the labelling agent coupled to a second reporter oligonucleotide.

In some aspects, these reporter oligonucleotides may comprise nucleic acid barcode sequences that permit identification of the labelling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being readily detected, e.g., using sequencing or array technologies.

Attachment (coupling) of the reporter oligonucleotides to the labelling agents may be achieved through any of a variety of direct or indirect, covalent or non-covalent associations or attachments. For example, oligonucleotides may be covalently attached to a portion of a labelling agent (such a protein, e.g., an antibody or antibody fragment) using chemical conjugation techniques (e.g., Lightning-Link® antibody labelling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry may be used to couple reporter oligonucleotides to labelling agents. Commercially available kits, such as those from Thunderlink and Abcam, and techniques common in the art may be used to couple reporter oligonucleotides to labelling agents as appropriate. In another example, a labelling agent is indirectly (e.g., via hybridization) coupled to a reporter oligonucleotide comprising a barcode sequence that identifies the label agent. For instance, the labelling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labelling agent to the reporter oligonucleotide. In some embodiments, the reporter oligonucleotides are releasable from the labelling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein. In some instances, the reporter oligonucleotides described herein may include one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, a sequencing primer or primer biding sequence (such as an R1, R2, or partial R1 or R2 sequence).

In some cases, the labelling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labelling agent (or reporter oligonucleotide) either directly or indirectly (e.g., the label can be conjugated to a molecule that can bind to the labelling agent or reporter oligonucleotide). In some cases, a label is conjugated to a first oligonucleotide that is complementary (e.g., hybridizes) to a sequence of the reporter oligonucleotide.

In some embodiments, multiple different species of analytes (e.g., polypeptides) from the biological sample can be subsequently associated with the one or more physical properties of the biological sample. For example, the multiple different species of analytes can be associated with locations of the analytes in the biological sample. Such information (e.g., proteomic information when the analyte binding moiety(ies) recognizes a polypeptide(s)) can be used in association with other spatial information (e.g., genetic information from the biological sample, such as DNA sequence information, transcriptome information (e.g., sequences of transcripts), or both). For example, a cell surface protein of a cell can be associated with one or more physical properties of the cell (e.g., a shape, size, activity, or a type of the cell). The one or more physical properties can be characterized by imaging the cell. The cell can be bound by an analyte labelling agent comprising an analyte binding moiety that binds to the cell surface protein and an analyte binding moiety barcode that identifies that analyte binding moiety. Results of protein analysis in a sample (e.g., a tissue sample or a cell) can be associated with DNA and/or RNA analysis in the sample.

(iii) Multiplexing (e.g., Endogenous Analyte and/or Labelling Agent)

In some embodiments, provided herein are methods and compositions for analyzing one or more endogenous analytes, products thereof, and/or a labelling agent in a biological sample. In some embodiments, multiplexing allows for detection of multiple analytes of interest (e.g., proteins, DNA, and/or RNA) as well as analyte interaction and co-localization. In some embodiments, this can be achieved by combining use of the polynucleotides described herein or in combination with other detection methods and reagents (e.g., labelling agents). In some embodiments, an endogenous analyte (e.g., a viral or cellular DNA or RNA) or a product (e.g., a hybridization product, a ligation product, an extension product (e.g., by a DNA or RNA polymerase), a replication product, a transcription/reverse transcription product, and/or an amplification product such as a rolling circle amplification (RCA) product) thereof is analyzed. In some embodiments, a labelling agent that directly or indirectly binds to an analyte in the biological sample is analyzed. In some embodiments, a product (e.g., a hybridization product, a ligation product, an extension product (e.g., by a DNA or RNA polymerase), a replication product, a transcription/reverse transcription product, and/or an amplification product such as a rolling circle amplification (RCA) product) of a labelling agent that directly or indirectly binds to an analyte in the biological sample is analyzed.

a. Hybridization

In some embodiments, a product of an endogenous analyte and/or a labelling agent is a hybridization product comprising the pairing of substantially complementary or complementary nucleic acid sequences within two different molecules, one of which is the endogenous analyte or the labelling agent (e.g., or an associated reporter oligonucleotide). The other molecule can be another endogenous molecule or another labelling agent such as a probe. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

Various probes and probe sets can be hybridized to an endogenous analyte and/or a labelling agent and each probe may comprise one or more barcode sequences. Exemplary barcoded probes or probe sets may be based on a padlock probe, a gapped padlock probe, a SNAIL (Splint Nucleotide Assisted Intramolecular Ligation) probe set, a PLAYR (Proximity Ligation Assay for RNA) probe set, a PLISH (Proximity Ligation in situ Hybridization) probe set, and RNA-templated ligation probes. The specific probe or probe set design can vary.

b. Ligation

In some embodiments, a product of an endogenous analyte and/or a labelling agent is a ligation product. In some embodiments, the ligation product is formed between two or more endogenous analytes. In some embodiments, the ligation product is formed between an endogenous analyte and a labelling agent. In some embodiments, the ligation product is formed between two or more labelling agent. In some embodiments, the ligation product is an intramolecular ligation of an endogenous analyte. In some embodiments, the ligation product is an intramolecular ligation of a labelling agent, for example, the circularization of a circularizable probe or probe set upon hybridization to a target sequence. The target sequence can be comprised in an endogenous analyte (e.g., nucleic acid such as genomic DNA or mRNA) or a product thereof (e.g., cDNA from a cellular mRNA transcript), or in a labelling agent (e.g., the reporter oligonucleotide) or a product thereof.

In some embodiments, provided herein is a probe or probe set capable of DNA-templated ligation, such as from a cDNA molecule. See, e.g., U.S. Pat. No. 8,551,710, which is hereby incorporated by reference in its entirety. In some embodiments, provided herein is a probe or probe set capable of RNA-templated ligation. See, e.g., U.S. Pat. Pub. 2020/0224244 which is hereby incorporated by reference in its entirety. In some embodiments, the probe set is a SNAIL probe set. See, e.g., U.S. Pat. Pub. 20190055594, which is hereby incorporated by reference in its entirety.

In some embodiments, the ligation herein is a proximity ligation of ligating two (or more) nucleic acid sequences that are in proximity with each other, e.g., through enzymatic means (e.g., a ligase). In some embodiments, proximity ligation can include a “gap-filling” step that involves incorporation of one or more nucleic acids by a polymerase, based on the nucleic acid sequence of a template nucleic acid molecule, spanning a distance between the two nucleic acid molecules of interest (see, e.g., U.S. Pat. No. 7,264,929, the entire contents of which are incorporated herein by reference). A wide variety of different methods can be used for proximity ligating nucleic acid molecules, including (but not limited to) “sticky-end” and “blunt-end” ligations. Additionally, single-stranded ligation can be used to perform proximity ligation on a single-stranded nucleic acid molecule. Sticky-end proximity ligations involve the hybridization of complementary single-stranded sequences between the two nucleic acid molecules to be joined, prior to the ligation event itself. Blunt-end proximity ligations generally do not include hybridization of complementary regions from each nucleic acid molecule because both nucleic acid molecules lack a single-stranded overhang at the site of ligation.

In some embodiments, provided herein is a multiplexed proximity ligation assay. See, e.g., U.S. Pat. Pub. 20140194311 which is hereby incorporated by reference in its entirety. In some embodiments, provided herein is a probe or probe set capable of proximity ligation, for instance a proximity ligation assay for RNA (e.g., PLAYR) probe set. See, e.g., U.S. Pat. Pub. 20160108458, which is hereby incorporated by reference in its entirety. In some embodiments, a circular probe can be indirectly hybridized to the target nucleic acid. In some embodiments, the circular construct is formed from a probe set capable of proximity ligation, for instance a proximity ligation in situ hybridization (PLISH) probe set. See, e.g., U.S. Pat. Pub. 2020/0224243 which is hereby incorporated by reference in its entirety.

In some embodiments, a probe such as a padlock probe may be used to analyze a reporter oligonucleotide, which may generated using proximity ligation or be subjected to proximity ligation. In some examples, the reporter oligonucleotide of a labelling agent that specifically recognizes a protein can be analyzed using in situ hybridization (e.g., sequential hybridization) and/or in situ sequencing (e.g., using padlock probes and rolling circle amplification of ligated padlock probes). Further, the reporter oligonucleotide of the labelling agent and/or a complement thereof and/or a product (e.g., a hybridization product, a ligation product, an extension product (e.g., by a DNA or RNA polymerase), a replication product, a transcription/reverse transcription product, and/or an amplification product) thereof can be recognized by another labelling agent and analyzed.

In some embodiments, an analyte (a nucleic acid analyte or non-nucleic acid analyte) can be specifically bound by two labelling agents (e.g., antibodies) each of which is attached to a reporter oligonucleotide (e.g., DNA) that can participate in ligation, replication, and sequence detection, analysis, and/or decoding reactions, e.g., using a probe or probe set (e.g. a padlock probe, a SNAIL probe set, a circular probe, or a padlock probe and a connector). In some embodiments, the probe set may comprise two or more probe oligonucleotides, each comprising a region that is complementary to each other. For example, a proximity ligation reaction can include reporter oligonucleotides attached to pairs of antibodies that can be joined by ligation if the antibodies have been brought in proximity to each other, e.g., by binding the same target protein (complex), and the DNA ligation products that form are then used to template amplification, as described for example in Soderberg et al., Methods. (2008), 45(3): 227-32, the entire contents of which are incorporated herein by reference. In some embodiments, a proximity ligation reaction can include reporter oligonucleotides attached to antibodies that each bind to one member of a binding pair or complex, for example, for analyzing a binding between members of the binding pair or complex. For detection of analytes using oligonucleotides in proximity, see, e.g., U.S. Patent Application Publication No. 2002/0051986, the entire contents of which are incorporated herein by reference. In some embodiments, two analytes in proximity can be specifically bound by two labelling agents (e.g., antibodies) each of which is attached to a reporter oligonucleotide (e.g., DNA) that can participate, when in proximity when bound to their respective targets, in ligation, replication, and/or sequence decoding reactions

In some embodiments, one or more reporter oligonucleotides (and optionally one or more other nucleic acid molecules such as a connector) aid in the ligation of the probe. Upon ligation, the probe may form a circularized probe. In some embodiments, one or more suitable probes can be used and ligated, wherein the one or more probes comprise a sequence that is complementary to the one or more reporter oligonucleotides (or portion thereof). The probe may comprise one or more barcode sequences. In some embodiments, the one or more reporter oligonucleotide may serve as a primer for rolling circle amplification (RCA) of the circularized probe. In some embodiments, a nucleic acid other than the one or more reporter oligonucleotide is used as a primer for rolling circle amplification (RCA) of the circularized probe. For example, a nucleic acid capable of hybridizing to the circularized probe at a sequence other than sequence(s) hybridizing to the one or more reporter oligonucleotide can be used as the primer for RCA. In other examples, the primer in a SNAIL probe set is used as the primer for RCA.

In some embodiments, one or more analytes can be specifically bound by two primary antibodies, each of which is in turn recognized by a secondary antibody each attached to a reporter oligonucleotide (e.g., DNA). Each nucleic acid molecule can aid in the ligation of the probe to form a circularized probe. In some instances, the probe can comprise one or more barcode sequences. Further, the reporter oligonucleotide may serve as a primer for rolling circle amplification of the circularized probe. The nucleic acid molecules, circularized probes, and RCA products can be analyzed using any suitable method disclosed herein for in situ analysis.

In some embodiments, the ligation involves chemical ligation. In some embodiments, the ligation involves template dependent ligation. In some embodiments, the ligation involves template independent ligation. In some embodiments, the ligation involves enzymatic ligation.

In some embodiments, the enzymatic ligation involves use of a ligase. In some aspects, the ligase used herein comprises an enzyme that is commonly used to join polynucleotides together or to join the ends of a single polynucleotide. An RNA ligase, a DNA ligase, or another variety of ligase can be used to ligate two nucleotide sequences together. Ligases comprise ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some embodiments, the ligase is a T4 RNA ligase. In some embodiments, the ligase is a splintR ligase. In some embodiments, the ligase is a single stranded DNA ligase. In some embodiments, the ligase is a T4 DNA ligase. In some embodiments, the ligase is a ligase that has an DNA-splinted DNA ligase activity. In some embodiments, the ligase is a ligase that has an RNA-splinted DNA ligase activity.

In some embodiments, the ligation herein is a direct ligation. In some embodiments, the ligation herein is an indirect ligation. “Direct ligation” means that the ends of the polynucleotides hybridize immediately adjacently to one another to form a substrate for a ligase enzyme resulting in their ligation to each other (intramolecular ligation). Alternatively, “indirect” means that the ends of the polynucleotides hybridize non-adjacently to one another, e.g., separated by one or more intervening nucleotides or “gaps”. In some embodiments, said ends are not ligated directly to each other, but instead occurs either via the intermediacy of one or more intervening (so-called “gap” or “gap-filling” (oligo)nucleotides) or by the extension of the 3′ end of a probe to “fill” the “gap” corresponding to said intervening nucleotides (intermolecular ligation). In some cases, the gap of one or more nucleotides between the hybridized ends of the polynucleotides may be “filled” by one or more “gap” (oligo)nucleotide(s) which are complementary to a splint, padlock probe, or target nucleic acid. The gap may be a gap of 1 to 60 nucleotides or a gap of 1 to 40 nucleotides or a gap of 3 to 40 nucleotides. In specific embodiments, the gap may be a gap of about 1, 2, 3, 4, 5, 6, 7, 8, 9 or 10 or more nucleotides, of any integer (or range of integers) of nucleotides in between the indicated values. In some embodiments, the gap between said terminal regions may be filled by a gap oligonucleotide or by extending the 3′ end of a polynucleotide. In some cases, ligation involves ligating the ends of the probe to at least one gap (oligo)nucleotide, such that the gap (oligo)nucleotide becomes incorporated into the resulting polynucleotide. In some embodiments, the ligation herein is preceded by gap filling. In other embodiments, the ligation herein does not require gap filling.

In some embodiments, ligation of the polynucleotides produces polynucleotides with melting temperature higher than that of unligated polynucleotides. Thus, in some aspects, ligation stabilizes the hybridization complex containing the ligated polynucleotides prior to subsequent steps, comprising amplification and detection.

In some aspects, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tₘ) of the DNA strands. This selectively reduces the concentration of annealed mismatched substrates (expected to have a slightly lower Tₘ around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

c. Primer Extension and Amplification

In some embodiments, a product is a primer extension product of an analyte, a labelling agent (e.g., or an associated reporter oligonucleotide), a probe or probe set bound to the analyte (e.g., a padlock probe bound to genomic DNA, mRNA, or cDNA), or a probe or probe set bound to the labelling agent (e.g., a padlock probe bound to one or more reporter oligonucleotides from the same or different labelling agents).

A primer is generally a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer, may in some cases, refer to a primer binding sequence. A primer extension reaction generally refers to any method where two nucleic acid sequences become linked (e.g., hybridized) by an overlap of their respective terminal complementary nucleic acid sequences (for example, 3′ termini). Such linking can be followed by nucleic acid extension (e.g., an enzymatic extension) of one, or both termini using the other nucleic acid sequence as a template for extension. Enzymatic extension can be performed by an enzyme including, but not limited to, a polymerase and/or a reverse transcriptase.

In some embodiments, a product of an endogenous analyte and/or a labelling agent is an amplification product of one or more polynucleotides, for instance, a circular probe or circularizable probe or probe set. In some embodiments, the amplifying is achieved by performing rolling circle amplification (RCA). In other embodiments, a primer that hybridizes to the circular probe or circularized probe is added and used as such for amplification. In some embodiments, the RCA comprises a linear RCA, a branched RCA, a dendritic RCA, or any combination thereof.

In some embodiments, the amplification is performed at a temperature between or between about 20° C. and about 60° C. In some embodiments, the amplification is performed at a temperature between or between about 30° C. and about 40° C. In some aspects, the amplification step, such as the rolling circle amplification (RCA) is performed at a temperature between at or about 25° C. and at or about 50° C., such as at or about 25° C., 27° C., 29° C., 31° C., 33° C., 35° C., 37° C., 39° C., 41° C., 43° C., 45° C., 47° C., or 49° C.

In some embodiments, upon addition of a DNA polymerase in the presence of appropriate dNTP precursors and other cofactors, a primer is elongated to produce multiple copies of the circular template. This amplification step can utilize isothermal amplification or non-isothermal amplification. In some embodiments, after the formation of the hybridization complex and association of the amplification probe, the hybridization complex is rolling-circle amplified to generate an amplicon (e.g., a DNA nanoball) containing multiple copies of the circular template or a sequence thereof. Techniques for rolling circle amplification (RCA) are known in the art such as linear RCA, a branched RCA, a dendritic RCA, or any combination thereof. (See, e.g., Baner et al, Nucleic Acids Research, 26:5073-5078, 1998; Lizardi et al, Nature Genetics 19:226, 1998; Mohsen et al., Acc Chem Res. 2016 Nov. 15; 49(11): 2540-2550; Schweitzer et al. Proc. Natl Acad. Sci. USA 97:101 13-1 19, 2000; Faruqi et al, BMC Genomics 2:4, 2000; Nallur et al, Nucl. Acids Res. 29:el 18, 2001; Dean et al. Genome Res. 1 1:1095-1099, 2001; Schweitzer et al, Nature Biotech. 20:359-365, 2002; U.S. Pat. Nos. 6,054,274, 6,291,187, 6,323,009, 6,344,329 and 6,368,801). Exemplary polymerases for use in RCA comprise DNA polymerase such phi29 (φ29) polymerase, Klenow fragment, Bacillus stearothermophilus DNA polymerase (BST), T4 DNA polymerase, T7 DNA polymerase, or DNA polymerase I. In some aspects, DNA polymerases that have been engineered or mutated to have desirable characteristics can be employed. In some embodiments, the polymerase is phi29 DNA polymerase.

In some aspects, during the amplification step, modified nucleotides can be added to the reaction to incorporate the modified nucleotides in the amplification product (e.g., nanoball). Exemplary of the modified nucleotides comprise amine-modified nucleotides. In some aspects of the methods, for example, for anchoring or cross-linking of the generated amplification product (e.g., nanoball) to a scaffold, to cellular structures and/or to other amplification products (e.g., other nanoballs). In some aspects, the amplification products comprises a modified nucleotide, such as an amine-modified nucleotide. In some embodiments, the amine-modified nucleotide comprises an acrylic acid N-hydroxysuccinimide moiety modification. Examples of other amine-modified nucleotides comprise, but are not limited to, a 5-Aminoallyl-dUTP moiety modification, a 5-Propargylamino-dCTP moiety modification, a N6-6-Aminohexyl-dATP moiety modification, or a 7-Deaza-7-Propargylamino-dATP moiety modification.

In some aspects, the polynucleotides and/or amplification product (e.g., amplicon) can be anchored to a polymer matrix. For example, the polymer matrix can be a hydrogel. In some embodiments, one or more of the polynucleotide probe(s) can be modified to contain functional groups that can be used as an anchoring site to attach the polynucleotide probes and/or amplification product to a polymer matrix. Exemplary modification and polymer matrix that can be employed in accordance with the provided embodiments comprise those described in, for example, WO 2014/163886, WO 2017/079406, US 2016/0024555, US 2018/0251833 and US 2017/0219465. In some examples, the scaffold also contains modifications or functional groups that can react with or incorporate the modifications or functional groups of the probe set or amplification product. In some examples, the scaffold can comprise oligonucleotides, polymers or chemical groups, to provide a matrix and/or support structures.

The amplification products may be immobilized within the matrix generally at the location of the nucleic acid being amplified, thereby creating a localized colony of amplicons. The amplification products may be immobilized within the matrix by steric factors. The amplification products may also be immobilized within the matrix by covalent or noncovalent bonding. In this manner, the amplification products may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products are resistant to movement or unraveling under mechanical stress.

In some aspects, the amplification products are copolymerized and/or covalently attached to the surrounding matrix thereby preserving their spatial relationship and any information inherent thereto. For example, if the amplification products are those generated from DNA or RNA within a cell embedded in the matrix, the amplification products can also be functionalized to form covalent attachment to the matrix preserving their spatial information within the cell thereby providing a subcellular localization distribution pattern. In some embodiments, the provided methods involve embedding the one or more polynucleotide probe sets and/or the amplification products in the presence of hydrogel subunits to form one or more hydrogel-embedded amplification products. In some embodiments, the hydrogel-tissue chemistry described comprises covalently attaching nucleic acids to in situ synthesized hydrogel for tissue clearing, enzyme diffusion, and multiple-cycle sequencing while an existing hydrogel-tissue chemistry method cannot. In some embodiments, to enable amplification product embedding in the tissue-hydrogel setting, amine-modified nucleotides are comprised in the amplification step (e.g., RCA), functionalized with an acrylamide moiety using acrylic acid N-hydroxysuccinimide esters, and copolymerized with acrylamide monomers to form a hydrogel.

In some embodiments, the RCA template may comprise the target analyte, or a part thereof, where the target analyte is a nucleic acid, or it may be provided or generated as a proxy, or a marker, for the analyte. As noted above, many assays are known for the detection of numerous different analytes, which use a RCA-based detection system, e.g., where the signal is provided by generating a RCP from a circular RCA template which is provided or generated in the assay, and the RCP is detected to detect the analyte. The RCP may thus be regarded as a reporter which is detected to detect the target analyte. However, the RCA template may also be regarded as a reporter for the target analyte; the RCP is generated based on the RCA template, and comprises complementary copies of the RCA template. The RCA template determines the signal which is detected, and is thus indicative of the target analyte. As will be described in more detail below, the RCA template may be a probe (e.g., a primary probe such as a padlock probe), or a part or component of a probe, or may be generated from a probe, or it may be a component of a detection assay (i.e. a reagent in a detection assay), which is used as a reporter for the assay, or a part of a reporter, or signal-generation system. The RCA template used to generate the RCP may thus be a circular (e.g. circularized) reporter nucleic acid molecule, namely from any RCA-based detection assay which uses or generates a circular nucleic acid molecule as a reporter for the assay. Since the RCA template generates the RCP reporter, it may be viewed as part of the reporter system for the assay.

In some embodiments, a product herein includes a molecule or a complex generated in a series of reactions, e.g., hybridization, ligation, extension, replication, transcription/reverse transcription, and/or amplification (e.g., rolling circle amplification), in any suitable combination. For example, a product comprising a target sequence for a probe disclosed herein may be a hybridization complex formed comprising a nucleic acid in a sample and an exogenously added nucleic acid probe. The exogenously added nucleic acid probe may comprise an overhang that does not hybridize to the nucleic acid but hybridizes to another probe. The exogenously added nucleic acid probe may be optionally ligated to a nucleic acid molecule or another exogenous nucleic acid molecule. In other examples, a product comprising a target sequence for a probe disclosed herein may be an RCP generated using a circularizable probe or probe set which hybridizes to a nucleic acid molecule (e.g., genomic DNA or mRNA) or product thereof (e.g., a transcript such as cDNA, a DNA-templated ligation product of two probes, or an RNA-templated ligation product of two probes). In other examples, a target sequence for a probe disclosed herein may be comprised by a probe (e.g., an intermediate probe such as a secondary probe) hybridized to an RCP. The probe may comprise an overhang that does not hybridize to the RCP but hybridizes to another probe. The probe may be optionally ligated to a nucleic acid molecule or another probe, e.g., an anchor probe that hybridize to the RCP.

C. Target Sequences

A target sequence for a probe (e.g., a circular probe formed by the polynucleotides disclosed herein) may be comprised in any analyte disclose herein, including an endogenous analyte (e.g., a viral or cellular nucleic acid), a labelling agent (e.g., an associated reporter oligonucleotide), or a product of an endogenous analyte and/or a labelling agent. In some embodiments, the target sequence is a DNA sequence.

In some aspects, one or more of the target sequences includes one or more barcode(s), e.g., at least two, three, four, five, six, seven, eight, nine, ten, or more barcodes. Barcodes can spatially-resolve molecular components found in biological samples, for example, within a cell or a tissue sample. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for identification and/or quantification of individual sequencing-reads (e.g., a barcode can be or can include a unique molecular identifier or “UMI”). In some aspects, a barcode comprises about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

In some embodiments, a barcode includes two or more sub-barcodes that together function as a single barcode. For example, a polynucleotide barcode can include two or more polynucleotide sequences (e.g., sub-barcodes) that are separated by one or more non-barcode sequences. In some embodiments, the one or more barcode(s) can also provide a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the polynucleotide.

In any of the preceding embodiments, barcodes (e.g., primary and/or secondary barcode sequences) can be analyzed (e.g., detected or sequenced) using any suitable methods or techniques, including those described herein, such as RNA sequential probing of targets (RNA SPOTs), sequential fluorescent in situ hybridization (seqFISH), single-molecule fluorescent in situ hybridization (smFISH), multiplexed error-robust fluorescence in situ hybridization (MERFISH), hybridization-based in situ sequencing (HybISS), in situ sequencing, targeted in situ sequencing, fluorescent in situ sequencing (FISSEQ), sequencing by synthesis (SBS), sequencing by ligation (SBL), sequencing by hybridization (SBH), or spatially-resolved transcript amplicon readout mapping (STARmap). In any of the preceding embodiments, the methods provided herein can include analyzing the barcodes by sequential hybridization and detection with a plurality of labelled probes (e.g., detection probes or oligonucleotides).

In some embodiments, in a barcode sequencing or analysis method, barcode sequences are detected for identification of other molecules including nucleic acid molecules (DNA or RNA) longer than the barcode sequences themselves, as opposed to direct sequencing of the longer nucleic acid molecules. In some embodiments, a N-mer barcode sequence comprises 4^{N} complexity given a sequencing read of N bases, and a much shorter sequencing read may be required for molecular identification compared to non-barcode sequencing methods such as direct sequencing. For example, 1024 molecular species may be identified using a 5-nucleotide barcode sequence (45=1024), whereas 8 nucleotide barcodes can be used to identify up to 65,536 molecular species, a number greater than the total number of distinct genes in the human genome. In some embodiments, the barcode sequences contained in the probes or RCPs are detected, rather than endogenous sequences, which can be an efficient read-out in terms of information per cycle of sequencing. Because the barcode sequences are pre-determined, they can also be designed to feature error detection and correction mechanisms, see, e.g., U.S. Pat. Pub. 20190055594 and WO2019199579A1, which are hereby incorporated by reference in their entirety.

### III. In Situ Sequence Analysis

In some embodiments, sequence analysis can be performed in situ. In situ sequence analysis methods are particularly useful, for example, when the biological sample remains intact after analytes on the sample surface (e.g., cell surface analytes) or within the sample (e.g., intracellular analytes) have been barcoded. In situ sequence analysis typically involves incorporation of a labeled nucleotide (e.g., fluorescently labeled mononucleotides or dinucleotides) in a sequential, template-dependent manner or hybridization of a labeled primer (e.g., a labeled random hexamer) to a nucleic acid template such that the identities (i.e., nucleotide sequence) of the incorporated nucleotides or labeled primer extension products can be determined, and consequently, the nucleotide sequence of the corresponding template nucleic acid. Aspects of in situ sequence analysis are described, for example, in Mitra et al., (2003) Anal. Biochem. 320, 55-65, and Lee et al., (2014) Science, 343(6177), 1360-1363, the entire contents of each of which are incorporated herein by reference.

In addition, examples of methods and systems for performing in situ sequencing are described in PCT Patent Application Publication Nos. WO2014/163886, WO2018/045181, WO2018/045186, U.S. Patent Application Publication Nos US2019/0177718, US2019/0194709, and in U.S. Pat. Nos. 10,138,509 and 10,179,932, the entire contents of each of which are incorporated herein by reference. Exemplary techniques for in situ sequence analysis include, but are not limited to, STARmap (described for example in Wang et al., (2018) Science, 361(6499) 5691), MERFISH (described for example in Moffitt, (2016) Methods in Enzymology, 572, 1-49), and FISSEQ (described for example in U.S. Patent Application Publication No. 2019/0032121). The entire contents of each of the foregoing references are incorporated herein by reference.

The composite padlock or circular probes disclosed herein can be used with a variety of techniques, including multiplexed in situ hybridization or in situ sequencing technology of an intact tissue or non-homogenized tissue. In some embodiments, the target nucleic acid is in a cell in a tissue. In some embodiments the tissue has been fixed and permeabilized.

Aspects of the invention include fixing intact tissue. The term “fixing” or “fixation” as used herein is the process of preserving biological material (e.g., tissues, cells, organelles, molecules, etc.) from decay and/or degradation. Fixation may be accomplished using any convenient protocol. Fixation can include contacting the sample with a fixation reagent (i.e., a reagent that contains at least one fixative). Samples can be contacted by a fixation reagent for a wide range of times, which can depend on the temperature, the nature of the sample, and on the fixative(s). For example, a sample can be contacted by a fixation reagent for 24 or less hours, 18 or less hours, 12 or less hours, 8 or less hours, 6 or less hours, 4 or less hours, 2 or less hours, 60 or less minutes, 45 or less minutes, 30 or less minutes, 25 or less minutes, 20 or less minutes, 15 or less minutes, 10 or less minutes, 5 or less minutes, or 2 or less minutes.

A sample can be contacted by a fixation reagent at various temperatures, depending on the protocol and the reagent used. For example, in some instances a sample can be contacted by a fixation reagent at a temperature ranging from −22° C. to 55° C., where specific ranges of interest include, but are not limited to 50 to 54° C., 40 to 44° C., 35 to 39° C., 28 to 32° C., 20 to 26° C., 0 to 6° C., and −18 to −22° C. In some instances a sample can be contacted by a fixation reagent at a temperature of −20° C., 4° C., room temperature (22-25° C.), 30° C., 37° C., 42° C., or 52° C.

Any convenient fixation reagent can be used. Common fixation reagents include crosslinking fixatives, precipitating fixatives, oxidizing fixatives, mercurials, and the like. Crosslinking fixatives chemically join two or more molecules by a covalent bond and a wide range of cross-linking reagents can be used. Examples of suitable cross liking fixatives include but are not limited to aldehydes (e.g., formaldehyde, also commonly referred to as “paraformaldehyde” and “formalin”; glutaraldehyde; etc.), imidoesters, NHS (N-Hydroxysuccinimide) esters, and the like. Examples of suitable precipitating fixatives include but are not limited to alcohols (e.g., methanol, ethanol, etc.), acetone, acetic acid, etc. In some embodiments, the fixative is formaldehyde (i.e., paraformaldehyde or formalin). A suitable final concentration of formaldehyde in a fixation reagent is 0.1 to 10%, 1-8%, 1-4%, 1-2%, 3-5%, or 3.5-4.5%, including about 1.6% for 10 minutes. In some embodiments the sample is fixed in a final concentration of 4% formaldehyde (as diluted from a more concentrated stock solution, e.g., 38%, 37%, 36%, 20%, 18%, 16%, 14%, 10%, 8%, 6%, etc.). In some embodiments the sample is fixed in a final concentration of 10% formaldehyde. In some embodiments the sample is fixed in a final concentration of 1% formaldehyde. In some embodiments, the fixative is glutaraldehyde. A suitable concentration of glutaraldehyde in a fixation reagent is 0.1 to 1%. A fixation reagent can contain more than one fixative in any combination. For example, in some embodiments the sample is contacted with a fixation reagent containing both formaldehyde and glutaraldehyde. In addition to the fixation methods described, tissue may be paraffin-embedded (e.g., FFPE), a frozen, or processed fresh tissue.

In some embodiments, the methods disclosed include embedding the sample in a hydrogel. The hydrogel-tissue chemistry described includes covalently attaching nucleic acids to in situ synthesized hydrogel for tissue clearing, enzyme diffusion, and multiple-cycle sequencing. For exemplary compositions, methodologies, and descriptions of hydrogels and processing hydrogel embedded tissues, see, e.g., U.S. Pat. Nos. 10,138,509; 10,545,075; 10,563,257; and PCT Patent App. PCT/US2019/025835, published as WO2019199579A1; each of which is hereby incorporated by reference in their entirety.

As used herein, the terms “hydrogel” or “hydrogel network” mean a network of polymer chains that are water-insoluble, sometimes found as a colloidal gel in which water is the dispersion medium. In other words, hydrogels are a class of polymeric materials that can absorb large amounts of water without dissolving. Hydrogels can contain over 99% water and may include natural or synthetic polymers, or a combination thereof.

Hydrogels also possess a degree of flexibility very similar to natural tissue, due to their significant water content. A detailed description of suitable hydrogels may be found in published U.S. patent application 20100055733, herein specifically incorporated by reference. As used herein, the terms “hydrogel subunits” or “hydrogel precursors” mean monomers (e.g., hydrophilic monomers), prepolymers, or polymers that can be crosslinked, or “polymerized”, to form a three-dimensional (3D) hydrogel network. Without being bound by any scientific theory, it is believed that embedding the biological specimen in the presence of hydrogel subunits crosslinks the components of the specimen to the hydrogel subunits, thereby securing molecular components in place, preserving the tissue architecture and cell morphology.

In some embodiments, the embedding includes copolymerizing the one or more amplicons with acrylamide. As used herein, the term “copolymer” describes a polymer which contains more than one type of subunit. The term encompasses polymer which include two, three, four, five, or six types of subunits.

Tissue specimens suitable for use with the methods described herein generally include any type of tissue specimens collected from living or dead subjects, such as, e.g., biopsy specimens and autopsy specimens, of which include, but are not limited to, epithelium, muscle, connective, and nervous tissue. Tissue specimens may be collected and processed using the methods described herein and subjected to microscopic analysis immediately following processing, or may be preserved and subjected to microscopic analysis at a future time, e.g., after storage for an extended period of time. In some embodiments, the methods described herein may be used to preserve tissue specimens in a stable, accessible and fully intact form for future analysis. In some embodiments, the methods described herein may be used to analyze a previously-preserved or stored tissue specimen. In some embodiments, the intact tissue includes brain tissue such as visual cortex slices. In some embodiments, the intact tissue is a thin slice with a thickness of 5-20 μm, including, but not limited to, e.g., 5-18 μm, 5-15 μm, or 5-10 μm. In other embodiments, the intact tissue is a thick slice with a thickness of 50-200 μm, including, but not limited to, e.g., 50-150 μm, 50-100 μm, or 50-80 μm.

The terms “permeabilization” or “permeabilize” as used herein refer to the process of rendering the cells (cell membranes etc.) of a sample permeable to experimental reagents such as nucleic acid probes, antibodies, chemical substrates, etc. Any convenient method and/or reagent for permeabilization can be used.

In some embodiments, the methods described herein support multiplexed spatial analysis. In some embodiments, spatial analysis of a biological analyte can be performed individually for each analyte of interest. In some embodiments, multiple biological analytes can be detected and spatially analyzed simultaneously within the same biological sample. In some embodiments, multiplexing (e.g., simultaneously detecting multiple markers) allows for examination of spatial arrangement of analytes of interest (e.g., proteins, DNA, RNA) as well as analyte interaction and co-localization thereby facilitating simultaneous analysis of multiple tissue markers. In some embodiments, multiple biological analytes can be detected and spatially analyzed at different times (e.g., detection and analysis of one category of analyte, followed by detection and analysis of another category of analyte, or detection and analysis of one analyte followed by detection and analysis of another analyte). For example, detection, examination, and analysis of one or more mRNA transcripts within a biological sample can occur prior to the detection, examination, and analysis of one or more mRNA transcripts.

In some embodiments, one or more of the barcodes disclosed herein can be correlated with the sequence complementary to the analyte, and thus a particular analyte. A number (n) of analytes can be examined by introducing (n) different sequences complementary to an analyte/barcode pluralities to the sample. In some embodiments, sequences complementary to an analyte can be used in multiplexed methods to analyze 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, 600, 700, 800, 900, 1000, or more analytes.

In some embodiments, methods provided herein include analyzing the barcode of the composite padlock or circular probes using multiplexed spatial imaging. In some embodiments, analyzing the barcode of the amplicons includes employing RNA sequential probing of targets (RNA SPOTs), sequential fluorescent in situ hybridization (seqFISH), single-molecule fluorescent in situ hybridization (smFISH), multiplexed error-robust fluorescence in situ hybridization (MERFISH), in situ sequencing, hybridization-based in situ sequencing (HybISS), targeted in situ sequencing, fluorescent in situ sequencing (FISSEQ), or spatially-resolved transcript amplicon readout mapping (STARmap). In some embodiments, the methods provided herein include analyzing the barcode of the composite padlock or circular probes by sequential hybridization and detection with a plurality of labelled probes. A variety of light-based sequencing technologies are known in the art. See, e.g., Landegren et al., Genome Res. 8:769-76 (1998); Kwok, Pharmocogenomics 1:95-100 (2000); and Shi, Clin. Chem. 47:164-172 (2001).

In some embodiments, the methods of the invention include the step of performing rolling circle amplification in the presence of a target nucleic acid of interest, wherein the performing includes contacting a target nucleic acid that comprises hybridization regions HR1′ and HR2′ with a first polynucleotide and a second polynucleotide to form a hybridization complex (see, e.g., FIGS. 1-4). In one embodiment, the first polynucleotide comprises a docking region DR1, a bridge region BR1, a hybridization region HR1, a barcode sequence BCa1, and a docking region DR1′ and the second polynucleotide comprises a docking region DR2, a bridge region BR2, a hybridization region HR2, one or both of barcode sequence BCb1 and a bridge region BR2′, and a docking region DR2′, where HR1 and HR2 hybridize to HR1′ and 11R2′, respectively. In one embodiment, the DR1/DR1′ hybridization and the DR2/DR2′ hybridization each forms a sticky end or a blunt end for ligation. Following ligation and thus circularization, an amplification primer is added, which binds to the hybridization complex, and is then used to amplify the circular probe.

The nature of the ligation reaction depends on the structural components of the polynucleotides used to form the padlock or circular probe. In one embodiment, the polynucleotides comprise complementary docking regions that self-assemble the two or more polynucleotides into a padlock probe that is either ready for ligation because no gaps exist between the docking regions, or is ready for a fill-in process, which will then permit the ligation of the polynucleotides to form the circular probe. In another embodiment, the docking regions are complementary to a splint primer. In one embodiment, the splint primer is complementary to one pair of docking regions of two polynucleotides. In another embodiment, the splint primer is complementary to two pairs of docking regions. In one aspect of this embodiment, the splint primer has two regions of complementarity to the docking regions of the polynucleotides that form the padlock probe. Typically, a splint probe of this embodiment will comprise a first docking region complementary sequence, a spacer, and a second docking region complementary sequence.

In some embodiments, ligation of the polynucleotides is achieved by adding ligase to the hybridization complex to generate a closed nucleic acid circle. In some embodiments, the adding ligase includes adding DNA ligase. The term “ligase” as used herein refers to an enzyme that is commonly used to join polynucleotides together or to join the ends of a single polynucleotide. Ligases include ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases include bacterial ligases such as E. coli DNA ligase and Taq DNA ligase, Ampligase® thermostable DNA ligase (Epicentre® Technologies Corp., part of Illumina®, Madison, Wis.) and phage ligases such as T3 DNA ligase, T4 DNA ligase and T7 DNA ligase and mutants thereof.

Other types of ligation are also contemplated for use with the disclosed methods. For example, the ligation reaction can be selected from the group consisting of enzymatic ligation, chemical ligation (e.g., click chemistry ligation), and template dependent ligation, or any combination thereof. The nature of the ligation reaction will determine the temperate at which the reaction is performed. In some embodiments, the ligation reaction is performed at a temperature lower than the temperature at which the hybridization complex is formed. In some embodiments, the ligation reaction is performed at a temperature lower than or similar to the melting temperature (Tₘ) of DR1/DR1′ hybridization, the Tₘ of DR2/DR2′ hybridization, and/or the Tₘ of DR1-DR2/DR1′-DR2′ hybridization. In some embodiments, the temperature at which the ligation reaction is performed is between about 10° C. and about 30° C., e.g., about 16° C.

Following formation of the circular probe, in some embodiments, an amplification primer is added. The amplification primer is complementary to the target nucleic acid and the circular probe. In some instances, such as where a splint is used to facilitate formation of the circular probe (see, e.g., FIG. 3 or FIG. 4), the splint may also function as an amplification primer. Washing steps can be performed at any point during the process to remove non-specifically bound probes, probes that have ligated, etc.

Upon addition of a DNA polymerase in the presence of appropriate dNTP precursors and other cofactors, the amplification primer is elongated by replication of multiple copies of the template. This amplification product can be readily detected by binding to a probe to one or more barcode sequences.

Amplification is next performed. The amplification step can utilize isothermal amplification or non-isothermal amplification. In some embodiments, after the formation of the hybridization complex and association of the amplification probe, the hybridization complex is rolling-circle amplified to generate a cDNA nanoball (i.e., amplicon) containing multiple copies of the cDNA. Techniques for rolling circle amplification (RCA) are known in the art such as linear RCA, a branched RCA, a dendritic RCA, or any combination thereof. (See, e.g., Baner et al, Nucleic Acids Research, 26:5073-5078, 1998; Lizardi et al, Nature Genetics 19:226, 1998; Schweitzer et al. Proc. Natl Acad. Sci. USA 97:101 13-1 19, 2000; Faruqi et al, BMC Genomics 2:4, 2000; Nallur et al, Nucl. Acids Res. 29:el 18, 2001; Dean et al. Genome Res. 1 1:1095-1099, 2001; Schweitzer et al, Nature Biotech. 20:359-365, 2002; U.S. Pat. Nos. 6,054,274, 6,291,187, 6,323,009, 6,344,329 and 6,368,801). In some embodiments the polymerase is phi29 DNA polymerase.

Following amplification, the sequence of the amplicon or a portion thereof, is determined, for example by sequencing, or imaging the amplicon. The sequencing can comprise sequencing by hybridization, sequencing by ligation, and/or fluorescent in situ sequencing, and/or wherein the in situ hybridization comprises sequential fluorescent in situ hybridization. The determination can also be achieved with the use of probes (e.g., probes used for sequencing by ligation).

In some embodiments, the one or more barcode sequences in the amplicon are contacted with probes configured to detect (e.g., through hybridization of labeled probes) the barcode sequence. In some embodiments, the probes are two or more nucleotides in length. In some embodiments, the probes are at or about two nucleotides in length. In some embodiments, the probes are at or about three nucleotides in length. In some embodiments, the probes are at or about four nucleotides in length. In some embodiments, the probes are at or about five nucleotides in length. In some embodiments, the probes are at or about five nucleotides in length. In some embodiments, the probes are at or about six nucleotides in length. In some embodiments, the probes are at or about seven nucleotides in length. In some embodiments, the probes are at or about eight nucleotides in length. In some embodiments, the probes are at or about nine nucleotides in length. In some embodiments, the probes are at or about 10 nucleotides in length. In some embodiments, the probes are at or about 12 nucleotides in length. In some embodiments, the probes are at or about 14 nucleotides in length. In some embodiments, the probes are at or about 16 nucleotides in length. In some embodiments, the probes are at or about 18 nucleotides in length. In some embodiments, the probes are at or about 20 nucleotides in length. In some embodiments, the probes are at or about 22 nucleotides in length. In some embodiments, the probes are at or about 24 nucleotides in length. In some embodiments, the probes are at or about 26 nucleotides in length. In some embodiments, the probes are at or about 28 nucleotides in length. In some embodiments, the probes are at or about 30 nucleotides in length. In some embodiments, the use of an 8-nt barcode would enable sequencing of the entire transcriptome. In some embodiments, the use of an 8-nt barcode would enable sequencing of 65,536 genes.

The probes can be labeled. In some embodiments, probe labels include fluorophores, isotopes, mass tags, or combinations thereof.

In some embodiments, the probes have sequences with formula NxByNz, wherein N is an unknown degenerate base; B is a known interrogatory base; and x, y, and z are integers independent of each other, wherein x and/or z equal zero or greater and y equals one or greater. In some embodiments, x and/or z equals 0. In some embodiments, x and/or z equals 1. In some embodiments, x and/or z equals 2. In some embodiments, x and/or z equals 4. In some embodiments, x and/or z equals 6. In some embodiments, x and/or z equals 8. In some embodiments, x and/or z equals 10. In some embodiments, x and/or z equals 12. In some embodiments, x and/or z equals 14. In some embodiments, x and/or z equals 16. In some embodiments, x and/or z equals 18. In some embodiments, x and/or z equals 20. In some embodiments, y equals 1. In some embodiments, y equals 2. In some embodiments, y equals 3. In some embodiments, y equals 4. In some embodiments, y equals 5. In some embodiments, y equals 6. In some embodiments, y equals 7. In some embodiments, y equals 8. In some embodiments, y equals 9. In some embodiments, y equals 10. In some embodiments, y equals 12. In some embodiments, y equals 14. In some embodiments, y equals 16. In some embodiments, y equals 18. In some embodiments, y equals 20.

In some aspects, the term “barcode” refers a label, or identifier, that conveys or is capable of conveying information (e.g., information about a target nucleic acid in a sample or a molecule such as a probe polynucleotide), such as a nucleic acid sequence that is used to identify a target nucleic acid, such as the presence of an RNA molecule. In some aspects, a barcode provides information for identification of the target nucleic acid.

Various methods can be used to detect nucleic acid sequences (e.g., a sequence of the first polynucleotide and/or second polynucleotide, or a product thereof). In some embodiments, detection of an RNA molecule is enabled by detection of nucleic acid sequence templated from the RNA or a cDNA of the RNA by sequencing by synthesis (SBS), sequencing by ligation (SBL), or sequencing by hybridization (SBH). In some embodiments, the detection of targeted RNA species is enabled by the detection of nucleic acid sequence contained in the probe or nucleic acid component of the probe complex by sequencing by synthesis (SBS), sequencing by ligation (SBL), or sequencing by hybridization (SBH); e.g. “barcode” sequencing. In some other embodiments, the detection of the targeted RNA species comprises detection of both nucleic acid sequence templated from RNA or cDNA and nucleic acid sequence contained in the probe or nucleic acid component of the probe complex, sequencing by synthesis (SBS), sequencing by ligation (SBL), or sequencing by hybridization (SBH).

In some embodiments, the barcode sequences comprise 4^{N} complexity given a sequencing read of N bases, and a much shorter sequencing read may be required for molecular identification by non-barcode sequencing methods such as direct sequencing of an RNA target or a cDNA. For example, 1024 molecular species may be identified using a 5-nucleotide barcode sequence (45=1024), whereas 8 nucleotide barcodes can be used to identify up to 65,536 molecular species, a number greater than the total number of distinct genes in the human genome. In some embodiments, the barcode sequences contained in the probes are detected, rather than endogenous sequences, which can be an efficient read-out in terms of information per cycle of sequencing. Because the barcode sequences are pre-determined, they can also be designed to feature error detection and correction mechanisms.

In some aspects, one or more of the polynucleotide probe(s) includes one or more barcode(s). In some aspects, at least two, three, four, five, six, seven, eight, nine, 10, or more barcodes are included in the padlock or circular probe formed of the plurality of polynucleotides.

The barcode sequencing methods disclosed herein can be applied to any sample from which spatial information is of interest. For example, the sample can be a biological sample, including a cell, a tissue, and a cellular matrix. Depending on the application, the biological sample can also be whole blood, serum, plasma, mucosa, saliva, cheek swab, urine, stool, cells, tissue, bodily fluid or a combination thereof.

Barcodes can spatially-resolve molecular components found in biological samples, for example, within a cell or a tissue sample. In some embodiments, a barcode includes two or more sub-barcodes that together function as a single barcode. For example, a polynucleotide barcode can include two or more polynucleotide sequences (e.g., sub-barcodes) that are separated by one or more non-barcode sequences.

In some embodiments, the one or more barcode(s) can also provide a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the polynucleotide.

In some embodiments, the composite padlock or circular probes herein are primary probes (e.g., ones that bind to a target mRNA molecule directly) and comprise one or more primary barcode sequences. In some embodiments, an amplification product (e.g., an RCA product) comprises multiple copies of the one or more primary barcode sequences or complementary sequences thereof, and the amplification product is detected using one or more detection probes (e.g., a detectably labeled oligo such as fluorescent oligonucleotides) that hybridize to the one or more primary barcode sequences or complementary sequences thereof.

In some embodiments, the method further comprises using one or more secondary probes that hybridize to the one or more primary barcode sequences or complementary sequences thereof on one or more primary probes. In some embodiments, the one or more secondary probes hybridize to an amplification product (e.g., an RCA product) comprising multiple copies of the one or more primary barcode sequences or complementary sequences thereof. In some embodiments, the one or more secondary probes comprise one or more secondary barcode sequences and are detected using one or more detection probes (e.g., a detectably labeled oligo such as fluorescent oligonucleotides) that hybridize to the one or more second barcode sequences or complementary sequences thereof.

In some embodiments, one or more barcodes of a probe are targeted by detectably labeled detection oligonucleotides, such as fluorescently labeled oligonucleotides. In some embodiments, one or more decoding schemes are used to decode the signals, such as fluorescence, for sequence determination. In any of the preceding embodiments, barcodes (e.g., primary and/or secondary barcode sequences) can be analyzed (e.g., detected or sequenced) using any suitable methods or techniques, including those described herein, such as RNA sequential probing of targets (RNA SPOTs), sequential fluorescent in situ hybridization (seqFISH), single-molecule fluorescent in situ hybridization (smFISH), multiplexed error-robust fluorescence in situ hybridization (MERFISH), in situ sequencing, hybridization-based in situ sequencing (HybISS), targeted in situ sequencing, fluorescent in situ sequencing (FISSEQ), or spatially-resolved transcript amplicon readout mapping (STARmap). In any of the preceding embodiments, the methods provided herein can include analyzing the barcodes by sequential hybridization and detection with a plurality of labelled probes (e.g., detection oligonucleotides). Exemplary decoding schemes are described in Eng et al., “Transcriptome-scale Super-Resolved Imaging in Tissues by RNA SeqFISH+,” Nature 568(7751):235-239 (2019); Chen et al., “Spatially resolved, highly multiplexed RNA profiling in single cells,” Science; 348(6233):aaa6090 (2015); Gyllborg et al., Nucleic Acids Res (2020) 48(19):e112; U.S. Pat. No. 10,457,980 B2; US 2016/0369329 A1; WO 2018/026873 A1; and US 2017/0220733 A1, all of which are incorporated by reference in their entirety. In some embodiments, these assays enable signal amplification, combinatorial decoding, and error correction schemes at the same time.

In some embodiments, nucleic acid hybridization can be used for sequencing. These methods utilize labeled nucleic acid decoder probes that are complementary to at least a portion of a barcode sequence. Multiplex decoding can be performed with pools of many different probes with distinguishable labels. Non-limiting examples of nucleic acid hybridization sequencing are described for example in U.S. Pat. No. 8,460,865, and in Gunderson et al., Genome Research 14:870-877 (2004).

In some embodiments, real-time monitoring of DNA polymerase activity can be used during sequencing. For example, nucleotide incorporations can be detected through fluorescence resonance energy transfer (FRET), as described for example in Levene et al., Science (2003), 299, 682-686, Lundquist et al., Opt. Lett. (2008), 33, 1026-1028, and term “perfectly et al., Proc. Natl. Acad. Sci. USA (2008), 105, 1176-1181.

In some aspects, the analysis and/or sequence determination can be carried out at room temperature for best preservation of tissue morphology with low background noise and error reduction. In some embodiments, the analysis and/or sequence determination comprises eliminating error accumulation as sequencing proceeds.

In some embodiments, the analysis and/or sequence determination involves washing to remove unbound polynucleotides, thereafter revealing a fluorescent product for imaging.

In some aspects, the detection (comprising imaging) is carried out using any of a number of different types of microscopy, e.g., confocal microscopy, two-photon microscopy, light-field microscopy, intact tissue expansion microscopy, and/or CLARITY™-optimized light sheet microscopy (COLM).

In some embodiments, fluorescence microscopy is used for detection and imaging of the detection probe. In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The “fluorescence microscope” comprises any microscope that uses fluorescence to generate an image, whether it is a more simple set up like an epifluorescence microscope, or a more complicated design such as a confocal microscope, which uses optical sectioning to get better resolution of the fluorescent image.

In some embodiments, confocal microscopy is used for detection and imaging of the detection probe. Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity—so long exposures are often required. As only one point in the sample is illuminated at a time, 2D or 3D imaging requires scanning over a regular raster (i.e., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. CLARITY™-optimized light sheet microscopy (COLM) provides an alternative microscopy for fast 3D imaging of large clarified samples. COLM interrogates large immunostained tissues, permits increased speed of acquisition and results in a higher quality of generated data.

Other types of microscopy that can be employed comprise bright field microscopy, oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECSTM), electrostatic force microscopy (EFM), fluidic force microscope (FuidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM), and intact tissue expansion microscopy (exM).

In some embodiments, provided herein is a method for analyzing a biological sample, the method comprising contacting the biological sample comprising a target nucleic acid with a first polynucleotide and a second polynucleotide to form a hybridization complex, wherein: the first polynucleotide comprises docking region DR1, hybridization region HR1, and docking region DR1′, the second polynucleotide comprises docking region DR2, hybridization region HR2, and docking region DR2′, the target nucleic acid comprises hybridization regions HR1′ and HR2′, and HR1 and HR2 hybridize to HR1′ and HR2′, respectively; the first polynucleotide comprises bridge region BR1 between DR1 and HR and/or the second polynucleotide comprises bridge region BR2 between DR2 and HR2; and the first polynucleotide comprises bridge region BR1′ between HR1 and DR1′ and/or the second polynucleotide comprises bridge region BR2′ between HR2 and DR2′. In some embodiments, DR1 and DR2 does not hybridize to the target nucleic acid in the hybridization complex. The hybridization complex can be formed at a temperature that is lower than the melting temperature of HR1/HR1′ hybridization and HR2/HR2′ hybridization. In some embodiments, for example as shown in FIG. 2, the hybridization complex is formed at a temperature higher than the melting temperature of DR1/DR1′ hybridization, DR2/DR2′ hybridization, or DR1-DR2/DR1′-DR2′ hybridization, such that when the first and second polynucleotides hybridize to the target nucleic acid (e.g., RNA), there is little or no DR1/DR1′ hybridization, DR2/DR2′ hybridization, or DR1-DR2/DR1′-DR2′ hybridization. In the example shown in FIG. 4, the hybridization complex is formed at a temperature higher than the melting temperature of DR1-DR2 hybridized to the splint or DR1′-DR2′ hybridized to the splint, such that when the first and second polynucleotides hybridize to the target nucleic acid (e.g., RNA), there is little or no splint hybridization. Once the polynucleotides are specifically hybridzed to the target nucleic acid, the sample may be brought to a lower temperature than the hybridization temperature, to allow docking regions to hybrize to one another or to a splint and subsequent ligation. One or more wash steps may be performed to remove non-specifically hybridized molecules.

In some embodiments, provided herein is a method for analyzing a biological sample, the method comprising: (a) contacting the biological sample comprising a target nucleic acid with a first polynucleotide and a second polynucleotide to form a hybridization complex, wherein: the first polynucleotide comprises docking region DR1, hybridization region HR1, and docking region DR1′, the second polynucleotide comprises docking region DR2, hybridization region HR2, and docking region DR2′, the target nucleic acid comprises hybridization regions HR1′ and HR2′, and HR1 and HR2 hybridize to HR1′ and HR2′, respectively, the first polynucleotide comprises bridge region BR1 between DR1 and HR1 and/or the second polynucleotide comprises bridge region BR2 between DR2 and HR2, and the first polynucleotide comprises bridge region BR1′ between HR1 and DR1′ and/or the second polynucleotide comprises bridge region BR2′ between HR2 and DR2′; wherein DR1 and DR2 does not hybridize to the target nucleic acid; (b) connecting DR1 to DR2 and DR1′ to DR2′, whereby the first polynucleotide and the second polynucleotide form a circular polynucleotide hybridized to the target nucleic acid; (c) performing rolling circle amplification in the presence of a primer that hybridizes to the circular polynucleotide, using the circular polynucleotide as a template for a polymerase to extend the primer and form an amplification product; and (d) detecting the amplification product in the biological sample. In some embodiments, the amplification product is generated and detected in situ. In some embodiments, the target nucleic acid and/or the amplification product is immobilized in the biological sample. In some embodiments, the target nucleic acid and/or the amplification product is crosslinked to one or more other molecules (e.g., a cellular molecule or an extracellular molecule) in the biological sample, a matrix such as a hydrogel, and/or one or more functional groups on a substrate. In some embodiments, the biological sample is a processed or cleared biological sample. In some embodiments, the biological sample is a tissue sample. In some embodiments, the tissue sample is a tissue slice between about 1 μm and about 50 μm in thickness, optionally wherein the tissue slice is between about 5 μm and about 35 μm in thickness. In some embodiments, the tissue sample is embedded in a hydrogel. In some embodiments, the amplification product is detected by sequential hybridization, sequencing by hybridization, sequencing by ligation, sequencing by synthesis, sequencing by binding, or a combination thereof.

### IV. Compositions and Kits

Also provided are compositions and kits, for example, comprising one or more polynucleotides (e.g., provided in Section I) of the polynucleotide probe set and reagents for performing the methods provided herein, for example, reagents required for one or more steps including hybridization, ligation, amplification, detection, sequencing, sample preparation, embedding and/or anchoring as described herein.

In some embodiments, provided herein is a hybridization complex comprising a first polynucleotide and a second polynucleotide, wherein: the first polynucleotide comprises docking region DR1, hybridization region HR1, and docking region DR1′, the second polynucleotide comprises docking region DR2, hybridization region HR2, and docking region DR2′, and the first and second polynucleotides are capable of hybridizing to a target nucleic acid comprising hybridization regions HR1′ and HR2′, wherein HR1 and HR2 are capable of hybridizing to HR1′ and HR2′, respectively, wherein the first polynucleotide comprises bridge region BR1 between DR1 and HR1 and/or the second polynucleotide comprises bridge region BR2 between DR2 and HR2; wherein the first polynucleotide comprises bridge region BR1′ between HR1 and DR1′ and/or the second polynucleotide comprises bridge region BR2′ between HR2 and DR2′; and DR1 is hybridized to DR1′ and DR2 is hybridized to DR2′, wherein DR1 and DR2 does not hybridize to the target nucleic acid; wherein the DR1/DR1′ hybridization and the DR2/DR2′ hybridization each forms a sticky end, and the two sticky ends are hybridized to each other. In some embodiments, the first and second polynucleotides are DNA molecules and the target nucleic acid is an RNA (e.g., mRNA) molecule. In some embodiments, the hybridization complex further comprises the target nucleic acid hybridized to the first and second polynucleotides.

In some embodiments, provided herein is a hybridization complex comprising a first polynucleotide, a second polynucleotide, and a splint, wherein: the first polynucleotide comprises docking region DR1, hybridization region HR1, and docking region DR1′, the second polynucleotide comprises docking region DR2, hybridization region HR2, and docking region DR2′, and the first and second polynucleotides are capable of hybridizing to a target nucleic acid comprising hybridization regions HR1′ and HR2′, wherein HR1 and HR2 are capable of hybridizing to HR1′ and HR2′, respectively; wherein the first polynucleotide comprises bridge region BR1 between DR1 and HR1 and/or the second polynucleotide comprises bridge region BR2 between DR2 and HR2; wherein the first polynucleotide comprises bridge region BR1′ between HR1 and DR1′ and/or the second polynucleotide comprises bridge region BR2′ between HR2 and DR2′; wherein DR1 and DR2 does not hybridize to the target nucleic acid; and wherein DR1, DR1′, DR2, and DR2′ are hybridized to the splint which comprises (1) a first region complementary to at least a portion of DR1′ and at least a portion of DR2′, and (2) a second region complementary to at least a portion of DR1 and at least a portion of DR2, optionally wherein the splint further comprises a spacer region between the first and second complementary regions. In some embodiments, the first and second polynucleotides and the splint are DNA molecules and the target nucleic acid is an RNA (e.g., mRNA) molecule. In some embodiments, the hybridization complex further comprises the target nucleic acid hybridized to the first and second polynucleotides.

In some embodiments, provided herein is a kit comprising a first polynucleotide and a second polynucleotide, wherein: the first polynucleotide comprises docking region DR1, hybridization region HR1, and docking region DR1′, the second polynucleotide comprises docking region DR2, hybridization region HR2, and docking region DR2′, and the first and second polynucleotides are capable of hybridizing to a target nucleic acid comprising hybridization regions HR1′ and HR2′, wherein HR1 and HR2 are capable of hybridizing to HR1′ and HR2′, respectively; wherein the first polynucleotide comprises bridge region BR1 between DR1 and HR1 and/or the second polynucleotide comprises bridge region BR2 between DR2 and HR2; wherein the first polynucleotide comprises bridge region BR1′ between HR1 and DR1′ and/or the second polynucleotide comprises bridge region BR2′ between HR2 and DR2′; wherein DR1 and DR2 does not hybridize to the target nucleic acid; and wherein DR1 is capable of hybridizing to DR1′ and DR2 is capable of hybridizing to DR2′, wherein the DR1/DR1′ hybridization and the DR2/DR2′ hybridization each forms a sticky end, and the two sticky ends comprise overhangs that are complementary to each other. In some embodiments, the first and second polynucleotides are DNA molecules and the target nucleic acid is an RNA (e.g., mRNA) molecule. In some embodiments, the first polynucleotide and/or the second polynucleotide comprises one or more barcode sequences.

In some embodiments, provided herein is a kit comprising a first polynucleotide and a second polynucleotide, wherein: the first polynucleotide comprises docking region DR1, hybridization region HR1, and docking region DR1′, the second polynucleotide comprises docking region DR2, hybridization region HR2, and docking region DR2′, the first and second polynucleotides are capable of hybridizing to a target nucleic acid comprising hybridization regions HR1′ and HR2′, wherein HR1 and HR2 are capable of hybridizing to HR1′ and HR2′, respectively; wherein the first polynucleotide comprises bridge region BR1 between DR1 and HR1 and/or the second polynucleotide comprises bridge region BR2 between DR2 and HR2; wherein the first polynucleotide comprises bridge region BR1′ between HR1 and DR1′ and/or the second polynucleotide comprises bridge region BR2′ between HR2 and DR2′; wherein DR1 and DR2 does not hybridize to the target nucleic acid; and wherein DR1, DR1′, DR2, and DR2′ are capable of hybridizing to a splint which comprises (1) a first region complementary to at least a portion of DR1′ and at least a portion of DR2′, and (2) a second region complementary to at least a portion of DR1 and at least a portion of DR2, optionally wherein the splint further comprises a spacer region between the first and second complementary regions. In some embodiments, the kit further comprises the splint. In some embodiments, the first and second polynucleotides and the splint are DNA molecules and the target nucleic acid is an RNA (e.g., mRNA) molecule. In some embodiments, the first polynucleotide, the second polynucleotide, and/or the splint comprises one or more barcode sequences.

The various components of the kit may be present in separate containers or certain compatible components may be precombined into a single container. In some embodiments, the kits further contain instructions for using the components of the kit to practice the provided methods.

In some embodiments, the kits can contain reagents and/or consumables required for performing one or more steps of the provided methods. In some embodiments, the kits contain reagents for embedding the biological sample. In some embodiments, the kits contain reagents, such as enzymes and buffers for ligation and/or amplification, such as ligases and/or polymerases. In some aspects, the kit can also include any of the reagents described herein, e.g., wash buffer, and ligation buffer. In some embodiments, the kits contain reagents for detection and/or sequencing, such as barcode detection probes or detectable labels. In some embodiments, the kits optionally contain other components, for example: nucleic acid primers, enzymes and reagents, buffers, nucleotides, modified nucleotides, reagents for additional assays.

### V. Terminology

Specific terminology is used throughout this disclosure to explain various aspects of the apparatus, systems, methods, and compositions that are described.

As used herein, the singular forms “a,” “an,” and “the” include plural referents unless the context clearly dictates otherwise. For example, “a” or “an” means “at least one” or “one or more.”

The term “about” as used herein refers to the usual error range for the respective value readily known to the skilled person in this technical field. Reference to “about” a value or parameter herein includes (and describes) embodiments that are directed to that value or parameter per se.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

### (i) Barcode

A “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes.

Barcodes can have a variety of different formats. For example, barcodes can include polynucleotide barcodes, random nucleic acid and/or amino acid sequences, and synthetic nucleic acid and/or amino acid sequences. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for identification and/or quantification of individual sequencing-reads (e.g., a barcode can be or can include a unique molecular identifier or “UMI”).

Barcodes can spatially-resolve molecular components found in biological samples, for example, at single-cell resolution (e.g., a barcode can be or can include a “spatial barcode”). In some embodiments, a barcode includes both a UMI and a spatial barcode. In some embodiments, a barcode includes two or more sub-barcodes that together function as a single barcode. For example, a polynucleotide barcode can include two or more polynucleotide sequences (e.g., sub-barcodes) that are separated by one or more non-barcode sequences.

### (ii) Nucleic Acid and Nucleotide

The terms “nucleic acid” and “nucleotide” are intended to be consistent with their use in the art and to include naturally-occurring species or functional analogs thereof. Particularly useful functional analogs of nucleic acids are capable of hybridizing to a nucleic acid in a sequence-specific fashion (e.g., capable of hybridizing to two nucleic acids such that ligation can occur between the two hybridized nucleic acids) or are capable of being used as a template for replication of a particular nucleotide sequence. Naturally-occurring nucleic acids generally have a backbone containing phosphodiester bonds. An analog structure can have an alternate backbone linkage including any of a variety of those known in the art. Naturally-occurring nucleic acids generally have a deoxyribose sugar (e.g., found in deoxyribonucleic acid (DNA)) or a ribose sugar (e.g. found in ribonucleic acid (RNA)).

A nucleic acid can contain nucleotides having any of a variety of analogs of these sugar moieties that are known in the art. A nucleic acid can include native or non-native nucleotides. In this regard, a native deoxyribonucleic acid can have one or more bases selected from the group consisting of adenine (A), thymine (T), cytosine (C), or guanine (G), and a ribonucleic acid can have one or more bases selected from the group consisting of uracil (U), adenine (A), cytosine (C), or guanine (G). Useful non-native bases that can be included in a nucleic acid or nucleotide are known in the art.

(iii) Probe and Target

A “probe” or a “target,” when used in reference to a nucleic acid or sequence of a nucleic acids, is intended as a semantic identifier for the nucleic acid or sequence in the context of a method or composition, and does not limit the structure or function of the nucleic acid or sequence beyond what is expressly indicated.

### (iv) Oligonucleotide and Polynucleotide

The terms “oligonucleotide” and “polynucleotide” are used interchangeably to refer to a single-stranded multimer of nucleotides from about 2 to about 500 nucleotides in length. Oligonucleotides can be synthetic, made enzymatically (e.g., via polymerization), or using a “split-pool” method. Oligonucleotides can include ribonucleotide monomers (i.e., can be oligoribonucleotides) and/or deoxyribonucleotide monomers (i.e., oligodeoxyribonucleotides). In some examples, oligonucleotides can include a combination of both deoxyribonucleotide monomers and ribonucleotide monomers in the oligonucleotide (e.g., random or ordered combination of deoxyribonucleotide monomers and ribonucleotide monomers). An oligonucleotide can be 4 to 10, 10 to 20, 21 to 30, 31 to 40, 41 to 50, 51 to 60, 61 to 70, 71 to 80, 80 to 100, 100 to 150, 150 to 200, 200 to 250, 250 to 300, 300 to 350, 350 to 400, or 400-500 nucleotides in length, for example. Oligonucleotides can include one or more functional moieties that are attached (e.g., covalently or non-covalently) to the multimer structure. For example, an oligonucleotide can include one or more detectable labels (e.g., a radioisotope or fluorophore).

### (v) Hybridizing, Hybridize, Annealing, and Anneal

The terms “hybridizing,” “hybridize,” “annealing,” and “anneal” are used interchangeably in this disclosure, and refer to the pairing of substantially complementary or complementary nucleic acid sequences within two different molecules. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

### (vi) Primer

A “primer” is a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer, may in some cases, refer to a primer binding sequence.

(vii) Primer Extension

A “primer extension” refers to any method where two nucleic acid sequences (e.g., a constant region from each of two distinct capture probes) become linked (e.g., hybridized) by an overlap of their respective terminal complementary nucleic acid sequences (i.e., for example, 3′ termini). Such linking can be followed by nucleic acid extension (e.g., an enzymatic extension) of one, or both termini using the other nucleic acid sequence as a template for extension. Enzymatic extension can be performed by an enzyme including, but not limited to, a polymerase and/or a reverse transcriptase.

(viii) Nucleic Acid Extension

A “nucleic acid extension” generally involves incorporation of one or more nucleic acids (e.g., A, G, C, T, U, nucleotide analogs, or derivatives thereof) into a molecule (such as, but not limited to, a nucleic acid sequence) in a template-dependent manner, such that consecutive nucleic acids are incorporated by an enzyme (such as a polymerase or reverse transcriptase), thereby generating a newly synthesized nucleic acid molecule. For example, a primer that hybridizes to a complementary nucleic acid sequence can be used to synthesize a new nucleic acid molecule by using the complementary nucleic acid sequence as a template for nucleic acid synthesis. Similarly, a 3′ polyadenylated tail of an mRNA transcript that hybridizes to a poly (dT) sequence (e.g., capture domain) can be used as a template for single-strand synthesis of a corresponding cDNA molecule.
(ix) Amplification

An “amplification” encompasses generating copies of genetic material, including DNA and RNA sequences. In a typical amplification, the reaction mixture includes the genetic material to be amplified, an enzyme, one or more primers that are employed in a primer extension reaction, and reagents for the reaction. The oligonucleotide primers are of sufficient length to provide for hybridization to complementary genetic material under annealing conditions. The length of the primers generally depends on the length of the amplification domains, but will typically be at least 4 bases, at least 5 bases, at least 6 bases, at least 8 bases, at least 9 bases, at least 10 base pairs (bp), at least 11 bp, at least 12 bp, at least 13 bp, at least 14 bp, at least 15 bp, at least 16 bp, at least 17 bp, at least 18 bp, at least 19 bp, at least 20 bp, at least 25 bp, at least 30 bp, at least 35 bp, and can be as long as 40 bp or longer, where the length of the primers will generally range from 18 to 50 bp. The genetic material can be contacted with a single primer or a set of two primers (forward and reverse primers), depending upon whether primer extension, linear or exponential amplification of the genetic material is desired.

In some embodiments, the amplification process uses a DNA polymerase enzyme. The DNA polymerase activity can be provided by one or more distinct DNA polymerase enzymes. In certain embodiments, the DNA polymerase enzyme is from a bacterium, e.g., the DNA polymerase enzyme is a bacterial DNA polymerase enzyme. For instance, the DNA polymerase can be from a bacterium of the genus Escherichia, Bacillus, Thermophilus, or Pyrococcus.

Suitable examples of DNA polymerases that can be used include, but are not limited to: E. coli DNA polymerase I, Bsu DNA polymerase, Bst DNA polymerase, Taq DNA polymerase, VENT™ DNA polymerase, DEEPVENT™ DNA polymerase, LongAmp® Taq DNA polymerase, LongAmp® Hot Start Taq DNA polymerase, Crimson LongAmp® Taq DNA polymerase, Crimson Taq DNA polymerase, OneTaq® DNA polymerase, OneTaq® Quick-Load® DNA polymerase, Hemo KlenTaq® DNA polymerase, REDTaq® DNA polymerase, Phusion® DNA polymerase, Phusion® High-Fidelity DNA polymerase, Platinum Pfx DNA polymerase, AccuPrime Pfx DNA polymerase, Phi29 DNA polymerase, Klenow fragment, Pwo DNA polymerase, Pfu DNA polymerase, T4 DNA polymerase and T7 DNA polymerase enzymes.

The term “DNA polymerase” includes not only naturally-occurring enzymes but also all modified derivatives thereof, including also derivatives of naturally-occurring DNA polymerase enzymes. For instance, in some embodiments, the DNA polymerase can have been modified to remove 5′-3′ exonuclease activity. Sequence-modified derivatives or mutants of DNA polymerase enzymes that can be used include, but are not limited to, mutants that retain at least some of the functional, e.g. DNA polymerase activity of the wild-type sequence. Mutations can affect the activity profile of the enzymes, e.g. enhance or reduce the rate of polymerization, under different reaction conditions, e.g. temperature, template concentration, primer concentration, etc. Mutations or sequence-modifications can also affect the exonuclease activity and/or thermostability of the enzyme.

In some embodiments, amplification can include reactions such as, but not limited to, a strand-displacement amplification reaction, a rolling circle amplification reaction, a ligase chain reaction, a transcription-mediated amplification reaction, an isothermal amplification reaction, and/or a loop-mediated amplification reaction.

In some embodiments, amplification uses a single primer that is complementary to the 3′ tag of target DNA fragments. In some embodiments, amplification uses a first and a second primer, where at least a 3′ end portion of the first primer is complementary to at least a portion of the 3′ tag of the target nucleic acid fragments, and where at least a 3′ end portion of the second primer exhibits the sequence of at least a portion of the 5′ tag of the target nucleic acid fragments. In some embodiments, a 5′ end portion of the first primer is non-complementary to the 3′ tag of the target nucleic acid fragments, and a 5′ end portion of the second primer does not exhibit the sequence of at least a portion of the 5′ tag of the target nucleic acid fragments. In some embodiments, the first primer includes a first universal sequence and/or the second primer includes a second universal sequence.

In some embodiments (e.g., when the amplification amplifies captured DNA), the amplification products can be ligated to additional sequences using a DNA ligase enzyme. The DNA ligase activity can be provided by one or more distinct DNA ligase enzymes. In some embodiments, the DNA ligase enzyme is from a bacterium, e.g., the DNA ligase enzyme is a bacterial DNA ligase enzyme. In some embodiments, the DNA ligase enzyme is from a virus (e.g., a bacteriophage). For instance, the DNA ligase can be T4 DNA ligase. Other enzymes appropriate for the ligation step include, but are not limited to, Tth DNA ligase, Taq DNA ligase, Thermococcus sp. (strain 9oN) DNA ligase (9oN™ DNA ligase, available from New England Biolabs, Ipswich, Mass.), and Ampligase™ (available from Epicentre Biotechnologies, Madison, Wis.). Derivatives, e.g. sequence-modified derivatives, and/or mutants thereof, can also be used.

In some embodiments, genetic material is amplified by reverse transcription polymerase chain reaction (RT-PCR). The desired reverse transcriptase activity can be provided by one or more distinct reverse transcriptase enzymes, suitable examples of which include, but are not limited to: M-MLV, MuLV, AMV, HIV, ArrayScript™, MultiScribe™, ThermoScript™, and SuperScript® I, II, III, and IV enzymes. “Reverse transcriptase” includes not only naturally occurring enzymes, but all such modified derivatives thereof, including also derivatives of naturally-occurring reverse transcriptase enzymes.

In addition, reverse transcription can be performed using sequence-modified derivatives or mutants of M-MLV, MuLV, AMV, and HIV reverse transcriptase enzymes, including mutants that retain at least some of the functional, e.g. reverse transcriptase, activity of the wild-type sequence. The reverse transcriptase enzyme can be provided as part of a composition that includes other components, e.g. stabilizing components that enhance or improve the activity of the reverse transcriptase enzyme, such as RNase inhibitor(s), inhibitors of DNA-dependent DNA synthesis, e.g. actinomycin D. Many sequence-modified derivative or mutants of reverse transcriptase enzymes, e.g. M-MLV, and compositions including unmodified and modified enzymes are commercially available, e.g. ArrayScript™, MultiScribe™, ThermoScript™, and SuperScript® I, II, III, and IV enzymes.

Certain reverse transcriptase enzymes (e.g. Avian Myeloblastosis Virus (AMV) Reverse Transcriptase and Moloney Murine Leukemia Virus (M-MuLV, MMLV) Reverse Transcriptase) can synthesize a complementary DNA strand using both RNA (cDNA synthesis) and single-stranded DNA (ssDNA) as a template. Thus, in some embodiments, the reverse transcription reaction can use an enzyme (reverse transcriptase) that is capable of using both RNA and ssDNA as the template for an extension reaction, e.g. an AMV or MMLV reverse transcriptase.

In some embodiments, the quantification of RNA and/or DNA is carried out by real-time PCR (also known as quantitative PCR or qPCR), using techniques well known in the art, such as but not limited to “TAQMAN™” or “SYBR®”, or on capillaries (“LightCycler® Capillaries”). In some embodiments, the quantification of genetic material is determined by optical absorbance and with real-time PCR. In some embodiments, the quantification of genetic material is determined by digital PCR. In some embodiments, the genes analyzed can be compared to a reference nucleic acid extract (DNA and RNA) corresponding to the expression (mRNA) and quantity (DNA) in order to compare expression levels of the target nucleic acids.

### (x) Antibody

An “antibody” is a polypeptide molecule that recognizes and binds to a complementary target antigen. Antibodies typically have a molecular structure shape that resembles a Y shape. Naturally-occurring antibodies, referred to as immunoglobulins, belong to one of the immunoglobulin classes IgG, IgM, IgA, IgD, and IgE. Antibodies can also be produced synthetically. For example, recombinant antibodies, which are monoclonal antibodies, can be synthesized using synthetic genes by recovering the antibody genes from source cells, amplifying into an appropriate vector, and introducing the vector into a host to cause the host to express the recombinant antibody. In general, recombinant antibodies can be cloned from any species of antibody-producing animal using suitable oligonucleotide primers and/or hybridization probes. Recombinant techniques can be used to generate antibodies and antibody fragments, including non-endogenous species.

Synthetic antibodies can be derived from non-immunoglobulin sources. For example, antibodies can be generated from nucleic acids (e.g., aptamers), and from non-immunoglobulin protein scaffolds (such as peptide aptamers) into which hypervariable loops are inserted to form antigen binding sites. Synthetic antibodies based on nucleic acids or peptide structures can be smaller than immunoglobulin-derived antibodies, leading to greater tissue penetration.

Antibodies can also include affimer proteins, which are affinity reagents that typically have a molecular weight of about 12-14 kDa. Affimer proteins generally bind to a target (e.g., a target protein) with both high affinity and specificity. Examples of such targets include, but are not limited to, ubiquitin chains, immunoglobulins, and C-reactive protein. In some embodiments, affimer proteins are derived from cysteine protease inhibitors, and include peptide loops and a variable N-terminal sequence that provides the binding site.

Antibodies can also refer to an “epitope binding fragment” or “antibody fragment,” which as used herein, generally refers to a portion of a complete antibody capable of binding the same epitope as the complete antibody, albeit not necessarily to the same extent. Although multiple types of epitope binding fragments are possible, an epitope binding fragment typically comprises at least one pair of heavy and light chain variable regions (VH and VL, respectively) held together (e.g., by disulfide bonds) to preserve the antigen binding site, and does not contain all or a portion of the Fc region. Epitope binding fragments of an antibody can be obtained from a given antibody by any suitable technique (e.g., recombinant DNA technology or enzymatic or chemical cleavage of a complete antibody), and typically can be screened for specificity in the same manner in which complete antibodies are screened. In some embodiments, an epitope binding fragment comprises an F(ab′)2 fragment, Fab′ fragment, Fab fragment, Fd fragment, or Fv fragment. In some embodiments, the term “antibody” includes antibody-derived polypeptides, such as single chain variable fragments (scFv), diabodies or other multimeric scFvs, heavy chain antibodies, single domain antibodies, or other polypeptides comprising a sufficient portion of an antibody (e.g., one or more complementarity determining regions (CDRs)) to confer specific antigen binding ability to the polypeptide.

### (xi) Affinity Group

An “affinity group” is a molecule or molecular moiety which has a high affinity or preference for associating or binding with another specific or particular molecule or moiety. The association or binding with another specific or particular molecule or moiety can be via a non-covalent interaction, such as hydrogen bonding, ionic forces, and van der Waals interactions. An affinity group can, for example, be biotin, which has a high affinity or preference to associate or bind to the protein avidin or streptavidin. An affinity group, for example, can also refer to avidin or streptavidin which has an affinity to biotin. Other examples of an affinity group and specific or particular molecule or moiety to which it binds or associates with include, but are not limited to, antibodies or antibody fragments and their respective antigens, such as digoxigenin and anti-digoxigenin antibodies, lectin, and carbohydrates (e.g., a sugar, a monosaccharide, a disaccharide, or a polysaccharide), and receptors and receptor ligands.

Any pair of affinity group and its specific or particular molecule or moiety to which it binds or associates with can have their roles reversed, for example, such that between a first molecule and a second molecule, in a first instance the first molecule is characterized as an affinity group for the second molecule, and in a second instance the second molecule is characterized as an affinity group for the first molecule.

(xii) Label, Detectable Label, and Optical Label

The terms “detectable label,” “optical label,” and “label” are used interchangeably herein to refer to a directly or indirectly detectable moiety that is associated with (e.g., conjugated to) a molecule to be detected, e.g., a probe for in situ assay, a capture probe or analyte. The detectable label can be directly detectable by itself (e.g., radioisotope labels or fluorescent labels) or, in the case of an enzymatic label, can be indirectly detectable, e.g., by catalyzing chemical alterations of a substrate compound or composition, which substrate compound or composition is directly detectable. Detectable labels can be suitable for small scale detection and/or suitable for high-throughput screening. As such, suitable detectable labels include, but are not limited to, radioisotopes, fluorophores, chemiluminescent compounds, bioluminescent compounds, and dyes.

The detectable label can be qualitatively detected (e.g., optically or spectrally), or it can be quantified. Qualitative detection generally includes a detection method in which the existence or presence of the detectable label is confirmed, whereas quantifiable detection generally includes a detection method having a quantifiable (e.g., numerically reportable) value such as an intensity, duration, polarization, and/or other properties. In some embodiments, the detectable label is bound to a feature or to a capture probe associated with a feature. For example, detectably labeled features can include a fluorescent, a colorimetric, or a chemiluminescent label attached to a bead (see, for example, Rajeswari et al., J. Microbiol Methods 139:22-28, 2017, and Forcucci et al., J. Biomed Opt. 10:105010, 2015, the entire contents of each of which are incorporated herein by reference).

In some embodiments, a plurality of detectable labels can be attached to a feature, capture probe, or composition to be detected. For example, detectable labels can be incorporated during nucleic acid polymerization or amplification (e.g., Cy5®-labelled nucleotides, such as Cy5®-dCTP). Any suitable detectable label can be used. In some embodiments, the detectable label is a fluorophore. For example, the fluorophore can be from a group that includes: 7-AAD (7-Aminoactinomycin D), Acridine Orange (+DNA), Acridine Orange (+RNA), Alexa Fluor® 350, Alexa Fluor® 430, Alexa Fluor® 488, Alexa Fluor® 532, Alexa Fluor® 546, Alexa Fluor® 555, Alexa Fluor® 568, Alexa Fluor® 594, Alexa Fluor® 633, Alexa Fluor® 647, Alexa Fluor® 660, Alexa Fluor® 680, Alexa Fluor® 700, Alexa Fluor® 750, Allophycocyanin (APC), AMCA/AMCA-X, 7-Aminoactinomycin D (7-AAD), 7-Amino-4-methylcoumarin, 6-Aminoquinoline, Aniline Blue, ANS, APC-Cy7, ATTO-TAG™ CBQCA, ATTO-TAG™ FQ, Auramine O-Feulgen, BCECF (high pH), BFP (Blue Fluorescent Protein), BFP/GFP FRET, BOBO™-1/BO-PRO™-1, BOBO™-3/BO-PRO™-3, BODIPY® FL, BODIPY® TMR, BODIPY® TR-X, BODIPY® 530/550, BODIPY® 558/568, BODIPY® 564/570, BODIPY® 581/591, BODIPY® 630/650-X, BODIPY® 650-665-X, BTC, Calcein, Calcein Blue, Calcium Crimson™, Calcium Green-1™ Calcium Orange™, Calcofluor® White, 5-Carboxyfluoroscein (5-FAM), 5-Carboxynaphthofluoroscein, 6-Carboxyrhodamine 6G, 5-Carboxytetramethylrhodamine (5-TAMRA), Carboxy-X-rhodamine (5-ROX), Cascade Blue®, Cascade Yellow™, CCF2 (GeneBLAzer™), CFP (Cyan Fluorescent Protein), CFP/YFP FRET, Chromomycin A3, Cl-NERF (low pH), CPM, 6-CR 6G, CTC Formazan, Cy2, Cy3®, Cy3.5, Cy5®, Cy5.5®, Cy7®, Cychrome (PE-Cy5), Dansylamine, Dansyl cadaverine, Dansylchloride, DAPI, Dapoxyl, DCFH, DHR, DiA (4-Di-16-ASP), DiD (DilC18(5)), DIDS, Dil (DilC18(3)), DiO (DiOC18(3)), DiR (DilC18(7)), Di-4 ANEPPS, Di-8 ANEPPS, DM-NERF (4.5-6.5 pH), DsRed (Red Fluorescent Protein), EBFP, ECFP, EGFP, ELF®-97 alcohol, Eosin, Erythrosin, Ethidium bromide, Ethidium homodimer-1 (EthD-1), Europium (III) Chloride, 5-FAM (5-Carboxyfluorescein), Fast Blue, Fluorescein-dT phosphoramidite, FITC, Fluo-3, Fluo-4, FluorX®, Fluoro-Gold™ (high pH), Fluoro-Gold™ (low pH), Fluoro-Jade, FM® 1-43, Fura-2 (high calcium), Fura-2/BCECF, Fura Red™ (high calcium), Fura Red™/Fluo-3, GeneBLAzer™ (CCF2), GFP Red Shifted (rsGFP), GFP Wild Type, GFP/BFP FRET, GFP/DsRed FRET, Hoechst 33342 & 33258, 7-Hydroxy-4-methylcoumarin (pH 9), 1,5 IAEDANS, Indo-1 (high calcium), Indo-1 (low calcium), Indodicarbocyanine, Indotricarbocyanine, JC-1, 6-JOE, JOJO™-1/JO-PRO™-1, LDS 751 (+DNA), LDS 751 (+RNA), LOLO™-1/LO-PRO™-1, Lucifer Yellow, LysoSensor™ Blue (pH 5), LysoSensor™ Green (pH 5), LysoSensor™ Yellow/Blue (pH 4.2), LysoTracker® Green, LysoTracker® Red, LysoTracker® Yellow, Mag-Fura-2, Mag-Indo-1, Magnesium Green™, Marina Blue®, 4-Methylumbelliferone, Mithramycin, MitoTracker® Green, MitoTracker® Orange, MitoTracker® Red, NBD (amine), Nile Red, Oregon Green® 488, Oregon Green® 500, Oregon Green® 514, Pacific Blue, PBF1, PE (R-phycoerythrin), PE-Cy5, PE-Cy7, PE-Texas Red, PerCP (Peridinin chlorphyll protein), PerCP-Cy5.5 (TruRed), PharRed (APC-Cy7), C-phycocyanin, R-phycocyanin, R-phycoerythrin (PE), PI (Propidium Iodide), PKH26, PKH67, POPO™-1/PO-PRO™-1, POPO™-3/PO-PRO™-3, Propidium Iodide (PI), PyMPO, Pyrene, Pyronin Y, Quantam Red (PE-Cy5), Quinacrine Mustard, R670 (PE-Cy5), Red 613 (PE-Texas Red), Red Fluorescent Protein (DsRed), Resorufin, RH 414, Rhod-2, Rhodamine B, Rhodamine Green™, Rhodamine Red™, Rhodamine Phalloidin, Rhodamine 110, Rhodamine 123, 5-ROX (carboxy-X-rhodamine), S65A, S65C, S65L, S65T, SBFI, SITS, SNAFL®-1 (high pH), SNAFL®-2, SNARF®-1 (high pH), SNARF®-1 (low pH), Sodium Green™, SpectrumAqua®, SpectrumGreen® #1, SpectrumGreen® #2, SpectrumOrange®, SpectrumRed, SYTO 11, SYTO® 13, SYTO® 17, SYTO® 45, SYTOX® Blue, SYTOX® Green, SYTOX® Orange, 5-TAMRA (5-Carboxytetramethylrhodamine), Tetramethylrhodamine (TRITC), Texas Red®/Texas Red®-X, Texas Red®-X (NHS Ester), Thiadicarbocyanine, Thiazole Orange, TOTO®-1/TO-PRO®-1, TOTO®-3/TO-PRO®-3, TO-PRO-5, Tri-color (PE-Cy5), TRITC (Tetramethylrhodamine), TruRed (PerCP-Cy5.5), WW 781, X-Rhodamine (XRITC), Y66F, Y66H, Y66W, YFP (Yellow Fluorescent Protein), YOYO®-1/YO-PRO®-1, YOYO®-3/YO-PRO®-3, 6-FAM (Fluorescein), 6-FAM (NHS Ester), 6-FAM (Azide), HEX, TAMRA (NHS Ester), Yakima Yellow, MAX, TET, TEX615, ATTO 488, ATTO 532, ATTO 550, ATTO 565, ATTO Rhol01, ATTO 590, ATTO 633, ATTO 647N, TYE 563, TYE 665, TYE 705, 5′ IRDye® 700, 5′ IRDye® 800, 5′ IRDye® 800CW (NHS Ester), WellRED D4 Dye, WellRED D3 Dye, WellRED D2 Dye, Lightcycler® 640 (NHS Ester), and Dy 750 (NHS Ester).

As mentioned above, in some embodiments, a detectable label is or includes a luminescent or chemiluminescent moiety. Common luminescent/chemiluminescent moieties include, but are not limited to, peroxidases such as horseradish peroxidase (HRP), soybean peroxidase (SP), alkaline phosphatase, and luciferase. These protein moieties can catalyze chemiluminescent reactions given the appropriate substrates (e.g., an oxidizing reagent plus a chemiluminescent compound. A number of compound families are known to provide chemiluminescence under a variety of conditions. Non-limiting examples of chemiluminescent compound families include 2,3-dihydro-1,4-phthalazinedione luminol, 5-amino-6,7,8-trimethoxy- and the dimethylamino[ca]benz analog. These compounds can luminesce in the presence of alkaline hydrogen peroxide or calcium hypochlorite and base. Other examples of chemiluminescent compound families include, e.g., 2,4,5-triphenylimidazoles, para-dimethylamino and -methoxy substituents, oxalates such as oxalyl active esters, p-nitrophenyl, N-alkyl acridinum esters, luciferins, lucigenins, or acridinium esters. In some embodiments, a detectable label is or includes a metal-based or mass-based label. For example, small cluster metal ions, metals, or semiconductors may act as a mass code. In some examples, the metals can be selected from Groups 3-15 of the periodic table, e.g., Y, La, Ag, Au, Pt, Ni, Pd, Rh, Ir, Co, Cu, Bi, or a combination thereof.

### EXAMPLES

The following examples are included for illustrative purposes only and are not intended to limit the scope of the invention.

### Example 1: Method for Profiling an Analyte Using a First and Second Oligonucleotide Probes

A biological sample is contacted with a plurality of oligonucleotides comprising a first oligonucleotide and a second oligonucleotide. The first oligonucleotide includes a docking region DR1, a bridge region BR1, a hybridization region HR1, one or more barcode sequences BCa1, . . . , BCai, where i is an whole number of 1 or greater, a bridge region BR1′, and a docking region DR1′, in the 5′ to 3′ direction or the 3′ to 5′ direction. HR1 hybridizes to a hybridization region HR1′ of a target mRNA molecule in the biological sample, and docking regions DR1 and DR1′ hybridize to each other. The second oligonucleotide includes a docking region DR2, a bridge region BR2, a hybridization region HR2, one or more barcode sequences BCb1, . . . , BCbj, where j is an whole number of 1 or greater, a bridge region BR2′, and a docking region DR2′, in the 3′ to 5′ direction (when the first oligonucleotide contains the described features in the 5′ to 3′ direction) or the 5′ to 3′ direction (when the first oligonucleotide contains the described features in the 3′ to 5′ direction). HR2 hybridizes to a hybridization region HR2′ of the target mRNA molecule in the biological sample, and docking regions DR2 and DR2′ hybridize to each other. Hybridization regions HR1′ and HR2′ are not contiguous on the target mRNA molecule. A hybridization complex containing the mRNA target molecule and the first and second oligonucleotides is formed, e.g., as shown in FIG. 2. The sample containing the hybridization complex is optionally washed.

DR1 and DR1′ are then ligated to DR2 and DR2′, respectively, to create a circular probe hybridized to the mRNA target. The ligation of DR1 and DR2 and the ligation of DR1′ and DR2′ are DNA-DNA templated ligation reactions. In some instances, the ligation reactions are performed at a lower temperature than the hybridization temperature. The sample containing the circular probe is optionally washed.

The circular probe is then amplified using a rolling circle amplification (RCA) primer hybridized to the circular probe and a phi29 polymerase. The sample containing the amplification product is optionally washed.

The amplification product is then subjected to in situ analysis in the biological sample. In some instances, the RCA products are sequenced with sequencing-by-ligation chemistry, for example, as described in Ke et al., “In situ sequencing for RNA analysis in preserved tissue and cells,” Nat. Methods 10, 857-860 (2013) or Wang et al., “Three-dimensional intact-tissue sequencing of single-cell transcriptional states,” Science 361, 380 (2018). A sequence of one or more of barcode sequences BCa1, . . . , BCai, and BCb1, . . . , BCbj is determined to provide information for identification of the target mRNA molecule. In some instances, the RCA products are analyzed using a sequential fluorescent in situ hybridization method, for example, as described in Eng et al., “Transcriptome-scale super-resolved imaging in tissues by RNA seqFISH+,” Nature 568(7751):235-239 (2019) and Chen et al., “Spatially resolved, highly multiplexed RNA profiling in single cells,” Science; 348(6233):aaa6090 (2015). The sample is imaged over multiple rounds of fluorescent probe hybridization, optionally one round of hybridization per barcode sequence in a single probe. In this manner, a sequential barcode is generated per imaged mRNA in a sample, allowing for the readout of the barcode sequences and the matching of each transcript to a target gene.

The assay can be multiplexed to analyze a plurality of amplification products from different mRNA molecules to spatially profile the transcriptome or a subset thereof in the biological sample.

### Example 2: Method for Profiling an Analyte Using a First and Second Probe Oligonucleotide and a Splint

A biological sample is contacted with a plurality of oligonucleotides comprising a first oligonucleotide and a second oligonucleotide. The first oligonucleotide includes a first barcode (e.g., BCa1), a second barcode (e.g., BCa2), a first docking sequence (e.g., DR1), a second docking sequence (e.g., DR1′), and a sequence complementary to the analyte (e.g., HR1 complementary to HR1′ of a target nucleic acid). The second oligonucleotide includes a third barcode (e.g., BCb1), a fourth barcode (e.g., BCb2), a third docking sequence (e.g., DR2), a fourth docking sequence (e.g., DR2′), and a sequence complementary to the analyte (e.g., HR2 complementary to HR2′ of the target nucleic acid). Additionally, the first oligonucleotide and the second oligonucleotide are complementary to a first sequence present in the analyte (e.g., HR1′) and a second sequence (e.g., HR2′) present in the analyte, respectively, and the first oligonucleotide and the second oligonucleotide each have an RNA-hybridizing portion and a DNA-hybridizing portion. The RNA-hybridizing portion of the first oligonucleotide is hybridized to the analyte (e.g., HR1 is hybridized to HR1′), and the RNA-hybridizing portion of the second oligonucleotide is hybridized to the analyte (e.g., HR2 is hybridized to HR2′), at a first temperature. A third oligonucleotide (e.g., a splint) is then hybridized to the docking sequences of the first oligonucleotide and the docking sequences of the second oligonucleotide at a second temperature that is lower than the first temperature. A portion of the third oligonucleotide (e.g., the splint) hybridizes to the first docking sequence (e.g., DR1) of the first oligonucleotide and the third docking sequence (e.g., DR2) of the second oligonucleotide and a portion of the third oligonucleotide (e.g., the splint) hybridizes to the second docking sequence (e.g., DR1′) of the first oligonucleotide and the fourth docking sequence (e.g., DR2′) of the second oligonucleotide. The first oligonucleotide and the second oligonucleotide are ligated at the second temperature to create a ligation product. See FIGS. 3-4, for example. All or a part of the sequence of the ligation product is determined. The determined sequence of the ligation product is used to spatially profile the analyte in the biological sample.

### Example 3: Method for Profiling an Analyte Using a First and Second Oligonucleotide Probes and a Splint

A biological sample is contacted with a plurality of oligonucleotides comprising a first oligonucleotide and a second oligonucleotide. The first oligonucleotide includes a docking region DR1, a bridge region BR1, a hybridization region HR1, one or more barcode sequences BCa1, . . . , BCai, where i is an whole number of 1 or greater, a bridge region BR1′, and a docking region DR1′, in the 5′ to 3′ direction or the 3′ to 5′ direction. HR1 hybridizes to a hybridization region HR1′ of a target mRNA molecule in the biological sample. The second oligonucleotide includes a docking region DR2, a bridge region BR2, a hybridization region HR2, one or more barcode sequences BCb1, . . . , BCbj, where j is an whole number of 1 or greater, a bridge region BR2′, and a docking region DR2′, in the 3′ to 5′ direction (when the first oligonucleotide contains the described features in the 5′ to 3′ direction) or the 5′ to 3′ direction (when the first oligonucleotide contains the described features in the 3′ to 5′ direction). HR2 hybridizes to a hybridization region HR2′ of the target mRNA molecule in the biological sample. Hybridization regions HR1′ and HR2′ are not contiguous on the target mRNA molecule. A hybridization complex containing the mRNA target molecule and the first and second oligonucleotides is formed. The sample containing the hybridization complex is optionally washed.

The sample is then contacted with a splint comprising (1) a first region complementary to at least a portion of DR1′ and at least a portion of DR2′; (2) a second region complementary to at least a portion of DR1 and at least a portion of DR2; and optionally a spacer between the first and second complementary regions. The docking regions are hybridized to the splint, e.g., as shown in FIG. 3B. The sample is optionally washed. DR1 and DR1′ are then ligated to DR2 and DR2′, respectively, to create a circular probe hybridized to the mRNA target. The ligation of DR1 and DR2 and the ligation of DR1′ and DR2′ are DNA-DNA templated ligation reactions. In some instances, the hybridization of the splint and/or the ligation reactions are performed at a lower temperature than the temperature at which the oligonucleotides hybridize to the target mRNA molecule. The sample containing the circular probe is optionally washed.

The circular probe is then amplified using a rolling circle amplification (RCA) primer hybridized to the circular probe and a phi29 polymerase. In some instances, the splint serves as the RCA primer. The sample containing the amplification product is optionally washed.

The amplification product is then subjected to in situ analysis in the biological sample. In some instances, the RCA products are sequenced with sequencing-by-ligation chemistry, for example, as described in Ke et al., “In situ sequencing for RNA analysis in preserved tissue and cells,” Nat. Methods 10, 857-860 (2013) or Wang et al., “Three-dimensional intact-tissue sequencing of single-cell transcriptional states,” Science 361, 380 (2018). A sequence of one or more of barcode sequences BCa1, . . . , BCai, and BCb1, . . . , BCbj is determined to provide information for identification of the target mRNA molecule. In some instances, the RCA products are analyzed using a sequential fluorescent in situ hybridization method, for example, as described in Eng et al., “Transcriptome-scale super-resolved imaging in tissues by RNA seqFISH+,” Nature 568(7751):235-239 (2019) and Chen et al., “Spatially resolved, highly multiplexed RNA profiling in single cells,” Science; 348(6233):aaa6090 (2015). The sample is imaged over multiple rounds of fluorescent probe hybridization, optionally one round of hybridization per barcode sequence in a single probe. In this manner, a sequential barcode is generated per imaged mRNA in a sample, allowing for the readout of the barcode sequences and the matching of each transcript to a target gene.

The assay can be multiplexed to analyze a plurality of amplification products from different mRNA molecules to spatially profile the transcriptome or a subset thereof in the biological sample.

### Example 4: Determining In Situ Gene Expression in Tissue Sections

In situ gene expression analysis is performed in tissue sections. Specifically, a tissue sample such as a mouse brain is surgically removal and without fixation, embedded into a medium such as an OCT (optimal cutting temperature) medium or the like, and directly frozen on dry ice. Thin sections, e.g., with a thickness of 10 μm, are cut with a cryostat and collected on glass slides. Sections are fixated, washed, and permeabilized. After permeabilization, sections are washed, and dehydrated, e.g., using an escalating ethanol series. Secure seal chambers are mounted on the slides to cover the tissue sections, and the sections are hydrated by a brief wash.

To target mRNAs with barcoded probes, sections are rehydrated and immersed in a hybridization mixture containing barcoded probes (e.g., the composite padlock or circular probes described in Examples 1-3), where the hybridization buffer optionally comprises saline-sodium citrate (SSC) buffer, formamide, KCl, bovine serume albumin (BSA), and/or RNAse inhibitor. Hybridization is optionally performed at 45° C. overnight and then washed.

For ligation, sections are immersed in a ligation mixture containing buffer, BSA, RNAse inhibitor, and T4 DNA ligase. Ligation is optionally performed for 60 minutes at 37° C. After ligation, the sections are optionally washed.

For rolling circle amplification (RCA), the sections are immersed in an RCA mixture containing phi29 polymerase buffer, dNTPs, BSA, phi29 polymerase, glycerol, and the RCA primer. RCA is optionally performed for three hours at 37° C. After RCA, the sections are optionally washed.

To determine in situ gene expression, sequential fluorescence in situ hybridization (seqFISH) or in situ sequencing is used to analyze the barcodes in the RCA products. The assay is multiplexed to analyze a plurality of amplification products from different mRNA molecules to spatially profile the transcriptome or a subset thereof in the tissue section.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

Having described some illustrative embodiments of the invention, it should be apparent to those skilled in the art that the foregoing is merely illustrative and not limiting, having been presented by way of example only. Numerous modifications and other illustrative embodiments are within the scope of one of ordinary skill in the art and are contemplated as falling within the scope of the invention. In particular, although many of the examples presented herein involve specific combinations of method acts or system elements, it should be understood that those acts and those elements may be combined in other ways to accomplish the same objectives.

1. A method for analyzing a target nucleic acid, comprising:
(a) contacting a target nucleic acid with a first polynucleotide and a second polynucleotide to form a hybridization complex, wherein:
the first polynucleotide comprises from either 5′ to 3′ or 3′ to 5′: docking region DR1, hybridization region HR1, a first bridge region, and docking region DR1′,
the second polynucleotide comprises from either 5′ to 3′ or 3′ to 5′: docking region DR2, hybridization region HR2, a second bridge region, and docking region DR2′,
the target nucleic acid comprises hybridization regions HR1′ and HR2′, and
HR1 and HR2 hybridize to HR1′ and HR2′, respectively;
wherein DR1 and DR2 do not hybridize to the target nucleic acid;
(b) hybridizing:
(i) DR1 to DR1′ and DR2 to DR2′, or
(ii) DR1, DR1′, DR2, and DR2′ to a splint comprising (1) a first region complementary to at least a portion of DR1′ and at least a portion of DR2′, and/or (2) a second region complementary to at least a portion of DR1 and at least a portion of DR2; and
(c) ligating DR1 to DR2 and DR1′ to DR2′ to connect the first polynucleotide and the second polynucleotide, thereby forming a circular polynucleotide hybridized to the target nucleic acid.

2. The method of claim 1, wherein the first polynucleotide and/or the second polynucleotide comprises one or more barcode sequences.

3. The method of claim 1, wherein DR1/DR1′ hybridization and DR2/DR2′ hybridization each forms a sticky end or a blunt end.

4. The method of claim 1, wherein the splint further comprises a spacer region between the first and second complementary regions.

5. (canceled)

6. The method of claim 1, wherein the first polynucleotide further comprises a first additional bridge region between DR1 and HR1 and/or the second polynucleotide comprises a second additional bridge region between DR2 and HR2.

7-16. (canceled)

17. The method of claim 2, wherein each barcode on the first polynucleotide and/or each barcode on the second polynucleotide, independent of one another, identifies the target nucleic acid or a sequence thereof, is a unique identifier of a gene, is an error-checking barcode, identifies an mRNA as a splice variant, and/or identifies a splice junction sequence.

18-27. (canceled)

28. The method of claim 1, wherein DR1 and DR2 form a first split hybridization region DR1-DR2, DR1′ and DR2′ form a second split hybridization region DR1′-DR2′, and DR1-DR2 and DR1′-DR2′ hybridize using each other as a splint.

29. The method claim 4, wherein the splint facilitates ligation of DR1 to DR2 and ligation of DR1′ to DR2′ and comprises (1) the first region complementary to a portion of DR1′ and a portion of DR2′, (2) the second region complementary to a portion of DR1 and a portion of DR2, and (3) the spacer region between the first and second complementary regions.

30-36. (canceled)

37. The method of claim 1, wherein the first polynucleotide and/or the second polynucleotide is a DNA molecule.

38-39. (canceled)

40. The method of claim 1, wherein the target nucleic acid is an mRNA.

41-42. (canceled)

43. The method of claim 1, wherein the melting temperature (Tₘ) of DR1/DR1′ hybridization, the Tₘ of DR2/DR2′ hybridization, and/or the Tₘ of DR1-DR2/DR1′-DR2′ hybridization are lower than the Tₘ of HR1/HR1′ hybridization and/or the Tₘ of HR2/HR2′ hybridization.

44. (canceled)

45. The method of claim 1, wherein the hybridization complex is formed at a temperature higher than the melting temperature (Tₘ) of DR1/DR1′ hybridization, the Tₘ of DR2/DR2′ hybridization, and/or the Tₘ of DR1-DR2/DR1′-DR2′ hybridization, but lower than the Tₘ of HR1/HR1′ hybridization and/or the Tₘ of HR2/HR2′ hybridization.

46-48. (canceled)

49. The method of claim 1, wherein formation of the circular polynucleotide comprises enzymatic ligation using a ligase having a DNA-splinted DNA ligase activity.

50-51. (canceled)

52. The method of claim 49, wherein the enzymatic ligation is performed at a temperature lower than or similar to the melting temperature (Tₘ) of DR1/DR1′ hybridization, the Tₘ of DR2/DR2′ hybridization, and/or the Tₘ of DR1-DR2/DR1′-DR2′ hybridization.

53. (canceled)

54. The method of claim 49, further comprising stringency wash after the enzymatic ligation.

55. (canceled)

56. The method of claim 1, further comprising forming an amplification product using the circular polynucleotide as a template.

57. The method of claim 4, further comprising forming an amplification product of the circular polynucleotide, wherein the splint is used as a primer for forming the amplification product, and the circular polynucleotide is used as a template for a polymerase to extend the splint and form the amplification product.

58. The method of claim 56, further comprising providing a primer for forming the amplification product, wherein the primer hybridizes to the circular polynucleotide, and the circular polynucleotide is used as a template for a polymerase to extend the primer and form the amplification product.

59-62. (canceled)

63. The method of claim 56, wherein a sequence in the amplification product is determined, and the sequence is indicative of the target nucleic acid or sequence thereof.

64-74. (canceled)

75. The method of claim 63, wherein the determination is performed when the target nucleic acid and/or the amplification product is in situ in a biological sample.

76-97. (canceled)
